(19) United States
(12) Patent Application Publication
(10) Pub. No.: US 2017/0276668 A1
(43) Pub. Date: Sep. 28, 2017

(54) NEURAL MICROPHYSIOLOGICAL SYSTEMS AND METHODS OF USING THE SAME

(71) Applicant: The Administrators of the Tulane Educational Fund, New Orleans, LA (US)

(72) Inventors: Jabe Lowry Curley, New Orleans, LA (US); Michael James Moore, New Orleans, LA (US); Parastoo Khoshakhlagh, Cambridge, MA (US); Benjamin John Hall, Bazel (CH)

(73) Assignee: The Administrators of the Tulane Educational Fund, New Orleans, LA (US)

(21) Appl. No.: 15/510,977

(22) PCT Filed: Sep. 14, 2015
(86) PCT No.: PCT/US15/50061

§ 371 Date: Mar. 13, 2017

Related U.S. Application Data
(60) Provisional application No. 62/049,692, filed on Sep. 12, 2014.
(60) Provisional application No. 62/138,258, filed on Mar. 25, 2015.

(51) Int. Cl. G01N 33/50; A61K 35/15; A61K 35/30

(52) U.S. Cl. CPC G01N 33/5058; A61K 35/30; A61K 35/15; G01N 33/5005

(57) ABSTRACT

The present disclosure generally relates to a cell culturing system, and specifically to a three-dimensional cell culturing system for neuronal cells that promotes both structural and functional characteristics that mimic those of in vivo peripheral fibers, including cell myelination. Using a dual hydrogel construct and explants from neuronal cells, the present disclosure provides methods, devices, and systems for in vitro spatially-controlled, three-dimensional models that permit intra- and extra-cellular electrophysiological measurements and recordings. The three-dimensional hydrogel constructs allow for flexibility in incorporated cell types, geometric fabrication, and electrical manipulation, providing viable systems for culture, perturbation, and testing of biomimetic neural growth with physiologically-relevant results.

### CROSS-REFERENCE TO RELATED APPLICATIONS

This application claims priority to U.S. Provisional Application No. 62/049,692 filed on Sep. 12, 2014, and U.S. Provisional Application No. 62/138,258 filed on Mar. 25, 2015, each of which is incorporated by reference in their entirety.

### FIELD

The present disclosure generally relates to a cell culturing system, and specifically to a three-dimensional cell culturing system for neuronal cells that promotes both structural and functional characteristics that mimic those of in vivo nerve fibers, including cell myelination and propagation of compound action potentials.

### BACKGROUND

Replicating functional aspects of physiology for bench top assessment is especially challenging for peripheral neuronal tissue, where bioelectrical conduction over long distances is one of the most relevant physiological outcomes. For this reason, three dimensional tissue models of peripheral nerves are lagging behind models of epithelial, metabolic, and tumor tissues, where soluble analytes serve as appropriate metrics. The application of electrophysiological techniques has recently been possible through multi-electrode array technologies for the screening of environmental toxins as well as for disease modeling and therapeutic testing. This application is groundbreaking for the study of both peripheral nervous system (PNS) and central nervous system (CNS) applications, but the dissociated nature of the cultures fails to replicate the population level environment and metrics critical for peripheral tissue. Instead, clinical methods of investigating peripheral neuropathy and neuroprotection include nerve conduction testing through measurement of compound action potentials (CAP) and nerve fiber density (NFD) using morphometric analysis of skin biopsies.

### SUMMARY

The present disclosure addresses a need to make and use a 3D hydrogel system that allows for in vitro physiological measurements of nerve tissue that mimics clinical nerve conduction and NFD.

The present disclosure relates to a method of producing a three-dimensional culture of one or a plurality of neuronal cells in a culture vessel comprising a solid substrate, said method comprising: (a) contacting one or a plurality of isolated Schwann cells and/or oligodendrocytes with the solid substrate, said substrate comprising at least one exterior surface, at least one interior surface and at least one interior chamber defined by the at least one interior surface and accessible from a point exterior to the solid substrate through at least one opening; (b) seeding one or a plurality of isolated neuronal cells or tissue explants comprising neuronal cells to the at least one interior chamber; (c) applying a cell medium into the culture vessel with a volume of cell medium sufficient to cover the at least one interior chamber; wherein at least one portion of the interior surface comprises a first cell-impenetrable polymer and a first cell-penetrable polymer. In some embodiments, step (a) is preceded by placing a solution comprising the first cell-impenetrable polymer and the first cell-penetrable polymer into the culture vessel and inducing the first cell-impenetrable polymer and the first cell-penetrable polymer to physically adhere or chemically bond onto at least a portion of the interior surface. In some embodiments, the solid substrate comprises a base with a predetermined shape that defines the shape of the exterior and interior surface.

In some embodiments, the base comprises one or a combination of silica, plastic, ceramic, or metal and wherein the base is in a shape of a cylinder or in a shape substantially similar to a cylinder, such that the first cell-impenetrable polymer and a first cell-penetrable polymer coat the interior surface of the base and define a cylindrical or substantially cylindrical interior chamber or compartment; and wherein the opening is positioned at one end of the cylinder. In some embodiments, the base comprises one or a plurality of pores of a size and shape sufficient to allow diffusion of protein, nutrients, and oxygen through the solid substrate in the presence of the cell culture medium.

In some embodiments, the step of inducing the first cell-impenetrable polymer and the first penetrable polymer to crosslink onto the solid substrate comprises exposing the solution to ultraviolet light or visible light. In some embodiments, the first cell-impenetrable polymer is polyethylene glycol (PEG) at a concentration of no more than about 20% weight to volume of the solution. In some embodiments, the first cell-penetrable polymer is at a concentration of from about 0.1% to about 3.0% in weight in volume of the solution.

In some embodiments, the method further comprises the step of exposing the culture vessel to 37° Celsius and a level of carbon dioxide of no more than about 5.0% for a time sufficient to allow growth of axons in the interior chamber. In some embodiments, at least one portion of the solid substrate is cylindrical or substantially cylindrical such that at least one portion of the interior surface of the solid substrate defines a cylindrical or substantially cylindrical interior chamber into which the one or plurality of Schwann cells are seeded and the one or plurality of neurons are seeded.

In some embodiments, step (c) comprises seeding tissue explants selected from one or a combination of: an isolated dorsal root ganglion, a spinal cord explant, a retinal explant, and a cortex explant. In some embodiments, step (c) comprises seeding a suspension of neuronal cells selected from one or a combination of: motor neurons, cortical neurons, spinal cord neurons, peripheral neurons.

In some embodiments, the solid substrate comprises a plastic base cross-linked with a mixture of the first cell-impenetrable polymer and the first cell-penetrable polymer; and wherein the plastic base comprises a plurality of pores with a diameter of no greater than about 1 micron.

In some embodiments, the method further comprises the step of forming a solid substrate and positioning said solid substrate in a culture vessel. In some embodiments, the step of forming a solid substrate comprises curing a solution comprising the first cell-impenetrable polymer and the first cell-penetrable polymer by photolithography.

In some embodiments, the method further comprises a step of allowing the neuronal cells to grow neurites and/or axons after step (c) for a period of from about 1 day to about 1 year.

In some embodiments, the method further comprises the step of isolating one or a plurality of Schwann cells and/or one or a plurality of oligodendrocytes from a sample prior to step (a).

In some embodiments, the method further comprises isolating dorsal root ganglion (DRG) from one or a plurality of mammals prior to step (b).

In some embodiments, the culture vessel is free of a sponge.

In some embodiments, the solid substrate comprises no greater than about 15% PEG and from about 0.05% to about 1.00% of one or a combination of self-assembling peptides chosen from: RAD 16-I, RAD 16-II, EAK 16-I, EAK 16-II, and of dEAK 16.

In some embodiments, the culture vessel comprises from about 1 to about 1200 wells into which steps (a)-(c) may be performed sequentially or simultaneously.

In some embodiments, at least a portion of the said substrate is formed in the shape of a cylinder or rectangular prism comprising an interior chamber defined by the inner surface and accessible by one or more openings.

In some embodiments, the solid substrate polymer is free of PEG.

In some embodiments, the cell medium comprises nerve growth factor (NGF) at a concentration from about 5 to about 20 picograms per milliliter and/or ascorbic acid in a concentration ranging from about 0.001% weight by volume to about 0.01% weight by volume.

In some embodiments, the method further comprises positioning at least one stimulating electrode at or proximate to soma of the one or plurality of neuronal cells or tissue explants and positioning at least one recording electrode at or proximate to an axon at a point most distal from the soma, such that. upon introducing a current in the stimulating electrode, the recording electrode is capable of receiving a signal corresponding to one or a plurality of electrophysiological metrics capable of being measured at the recording electrode. In some embodiments, the one or plurality of electrophysiological metrics are one or a combination of: electrical conduction velocity, action potential, amplitude of the wave associated with passage of an electrical impulse along a membrane of one or a plurality of neuronal cells, a width of an electrical impulses along a membrane of one or a plurality of neuronal cells, latency of the electrical impulse along a membrane of one or a plurality of neuronal cells, and envelope of the electrical impulse along a membrane of one or a plurality of neuronal cells.

The present disclosure also relates to a composition comprising: (i) a culture vessel; a hydrogel matrix comprising at least a first cell-impenetrable polymer and a first cell-penetrable polymer; and one or a plurality of isolated Schwann cells and/or one or a plurality of oligodendrocytes; and one or a plurality of tissue explants or fragments thereof; or (ii) a culture vessel; a hydrogel matrix comprising at least a first cell-impenetrable polymer and a first cell-penetrable polymer; and one or a plurality of isolated Schwann cells and/or one or a plurality of oligodendrocytes; and a suspension of cells comprising one or a plurality of neuronal cells.

In some embodiments, the composition further comprises a solid substrate onto which the hydrogel matrix is crosslinked, said solid substrate comprising at least one predominantly plastic surface with pores from about 1 micron to about 5 microns in diameter. In some embodiments, the composition further comprises a solid substrate onto which the hydrogel matrix is crosslinked, said solid substrate comprising at least one exterior surface and at least one interior surface and at least one interior chamber defined by the at least one interior surface and accessible from a point exterior to the solid substrate through at least one opening. In some embodiments, the composition further comprises a cell culture medium and/or cerebral spinal fluid.

In some embodiments, the tissue explants or fragments thereof are one or a combination of: DRG explants, retinal tissue explants, cortical explants, spinal cord explants, and peripheral nerve explants.

In some embodiments, the composition further comprises a solid substrate with a contiguous exterior surface and an interior surface, such solid substrate comprising at least one portion in a cylindrical or substantially cylindrical shape and at least one hollow interior defined at its edge by at least one portion of the interior surface, said interior surface comprising one or a plurality of pores from about 0.1 microns to about 1.0 microns in diameter wherein the hollow interior of the solid substrate is accessible from a point exterior to the solid substrate through at least one opening; wherein the hollow interior portion comprises a first portion proximate to the opening and at least a second portion distal to the opening; wherein the one or plurality of neuronal cells and/or the one or plurality of tissue explants are positioned at or proximate to the first portion of the hollow interior and are in physical contact with the hydrogel matrix, and wherein the second portion of the at least one hollow interior is in fluid communication with the first portion such that axons are capable of growth from the one or plurality of neuronal cells and/or the one or plurality of tissue explants into the second interior portion of the hollow interior.

In some embodiments, the composition is free of a sponge.

In some embodiments, the at least one cell-impenetrable polymer comprises no greater than about 15% PEG and the at least one cell-penetrable polymer comprises from about 0.05% to about 1.00% of one or a combination of self-assembling peptides chosen from: RAD 16-I, RAD 16-II, EAK 16-I, EAK 16-II, and dEAK 16.

In some embodiments, the culture vessel comprises 96, 192, 384 or more interior chambers in which one or plurality of isolated Schwann cells and/or one or plurality of oligodendrocytes are sufficiently proximate to the one or plurality of isolated tissue explants and/or the one or plurality of neuronal cells such that the Schwann cells or the oligodendrocytes deposit myelin to axon growth from the tissue explants and/or neuronal cells.

In some embodiments, the solid substrate is free of PEG.

In some embodiments, at least a portion of the said substrate is formed in the shape of a cylinder or rectangular prism comprising a space defined by the inner surface and accessible by one or more openings.

In some embodiments, the composition further comprises a cell medium comprising nerve growth factor (NGF) at a concentration from about 5 to about 20 picograms per milliliter and/or ascorbic acid in a concentration ranging from about 0.001% weight by volume to about 0.01% weight by volume.

In some embodiments, the one or more neuronal cells comprises at least one cell selected from the group comprising a glial cell, an embryonic cell, a mesenchymal stem cell, and a cell derived from an induced pluripotent stem cells. In some embodiments, the composition further comprises one or a plurality of stem cells or pluripotent cells. In some embodiments, the one or more neuronal cells comprises a primary mammalian cell derived from the peripheral nervous system of the mammal.

In some embodiments, the hydrogel matrix comprises at least 1% polyethylene glycol (PEG).

In some embodiments, the neuronal cells and/or tissue explants are in culture for no less than 3, 30, 90, or 365 days.

In some embodiments, at least one portion of the solid substrate is cylindrical or substantially cylindrical such that at least one portion of the interior surface of the solid substrate defines a cylindrical or substantially cylindrical hollow interior chamber in which the one or plurality of Schwann cells and the one or plurality of neurons in contact.

In some embodiments, the one or plurality of tissue explants comprises one or a plurality of DRGs with axonal growth from about 100 microns to about 500 microns in width and from about 0.11 to about 10000 microns in length.

In some embodiments, the composition further comprises at least two electrodes in operable communication with an electrochemical cell and a voltmeter, wherein a first stimulating electrode is positioned at or proximate to soma of the tissue explant and a second recording electrode is positioned at or proximate to a distal end of an axon such that the electrodes create a voltage difference along a distance of membrane of at least one cell in the tissue explant.

The present disclosure also relates to a method of assessing a response from one or more neuronal cells comprising: growing one or more neuronal cells in a culture vessel; introducing one or more stimuli to the one or more neuronal cells; and measuring one or more responses from the one or more neuronal cells to the one or more stimuli. In some embodiments, the one or more neuronal cells comprise sensory peripheral neurons. In some embodiments, the one or more neuronal cells comprise at least one or a combination of cells chosen from: spinal motor neurons, sympathetic neurons, and central nervous system (CNS) neurons.

In some embodiments, the culture vessel comprise a hydrogel matrix crosslinked to a solid substrate with a predetermined shape and wherein the hydrogel matrix comprises at least one cell-impenetrable polymer and at least one cell-penetrable polymer. In some embodiments, the hydrogel matrix comprises one or a combination of compounds chosen from: Puramatrix, methacrylated hyaluronic acid, agarose, methacrylated heparin, and methacrylated dextran.

In some embodiments, the one or more stimuli comprise an electrical current and the one or more responses comprise electrophysiological metrics. In some embodiments, the responses are measured by an optical recording technique.

In some embodiments, the one or more stimuli comprise one or a combination of: one or a plurality of optogenetic actuators, one or a plurality of caged neurotransmitters, one or a plurality of infrared lasers, or one or a plurality of light-gated ion-channels.

In some embodiments, the step of measuring comprises monitoring the movement of voltage-sensitive dyes, calcium dyes, or using label-free photonic imaging. In some embodiments, the one or more neuronal cells comprise isolated primary ganglion tissue.

In some embodiments, at least a portion of the solid substrate is micropatterned by photolithography and comprises an exterior surface, an interior surface, and at least one interior chamber defined by the at least one interior surface; wherein the method further comprising seeding the one or more neuronal cells in such micropatterned solid substrate such that growth the one or more neuronal cells is confined to specific geometries defined by the at least one interior chamber. In some embodiments, the interior chamber separates cell bodies from axonal processes in distinct locations. In some embodiments, the shape of the interior chamber allows for interrogation of any of the morphometric or electrophysiological metrics to be detecting and used in separate locations within the chamber. Typically, for instance, the interior chamber or interior compartment of the solid substrate of the hydrogel matrix, if a solid substrate is not being used, allows for one or a plurality of locations within the matrix or substrate to address cell bodies and axonal processes in distinct locations.

In some embodiments, the one or more neuronal cells are derived from primary human tissue or from human stem cells. In some embodiments, the one or more neuronal cells are primary mammalian neurons. In some embodiments, the at least one neuronal cells comprises an isolated DRG or fragment thereof; and inducing a stimulus from the one or more neuronal cells comprises placing a stimulating electrodes at or proximate to cell soma of the DRG or fragment thereof and placing a recording electrode at or proximate to an axonal process most distal to the soma.

In some embodiments, the one or more stimuli comprise an electrical or chemical stimulus. In some embodiments, the one or more stimuli comprises contacting the one or more neuronal cells and/or the one or plurality of tissue explants with at least one pharmacologically active compound

The present disclosure also relates to a method of evaluating the toxicity of an agent comprising: (a) culturing one or more neuronal cells and/or one or more tissue explants in any of the compositions disclosed herein; (b) exposing at least one agent to the one or more neuronal cells and/or one or more tissue explants; (c) measuring and/or observing one or more morphometric changes of the one or more neuronal cells and/or one or more tissue explants; and (d) correlating one or more morphometric changes of the one or more neuronal and/or one or more tissue explants cells with the toxicity of the agent, such that, if the morphometric changes are indicative of decreased cell viability, the agent is characterized as toxic and, if the morphometric changes are indicative of unchanged or increased cell viability, the agent is characterized as non-toxic.

The present disclosure also relates to a method of evaluating the relative degree of toxicity of a first agent as compared to a second agent comprising: (a) culturing one or more neuronal cells and/or one or more tissue explants in any of the compositions disclosed herein; (b) exposing a first agent and a second agent to the one or more neuronal cells and/or one or more tissue explants in sequence or in parallel time periods (in sequence if on the same set of cells or in parallel if on a second set of cells—for instance, in a multiplexed system); (c) measuring and/or observing one or more morphometric changes of the one or more neuronal cells and/or one or more tissue explants; and (d) correlating one or more morphometric changes of the one or more neuronal and/or one or more tissue explants cells with the toxicity of the first agent; and (e) correlating one or more morphometric changes of the one or more neuronal and/or one or more tissue explants cells with the toxicity of the second agent; and (f) comparing the toxicities of the first and second agent; and (g) characterizing the first or second agent as more toxic or less toxic than the second agent. In some embodiments, when characterizing the first or second agent as more toxic or less toxic than the second agent, if the morphometric changes induced by the first agent are more severe and indicative of decreased cell viability to a greater extent than the second compound, the first agent is more toxic than the second agent; and, if the morphometric changes induced by the first agent are less severe and/or indicative of increased cell viability as compared to the second compound, then the second agent is more toxic than the first agent. The same characterization can be applied in embodiments in which electrophysiological metrics are observed and/or measured.

In some embodiments, the degree of toxicity is determined by repeating any one or more of the steps provided herein with one or a series of doses or amounts of an agent. Rather than comparing or contrasting the relative toxicities among two different agents, one of skill in the art can this way add varying doses of the same agent to characterize when and at what dose the agent may become toxic to the one or plurality of neurons.

The present disclosure also relates to a method of evaluating the toxicity of an agent comprising: (a) culturing one or more neuronal cells and/or one or more tissue explants in any of the compositions disclosed herein; (b) exposing at least one agent to the one or more neuronal cells and/or one or more tissue explants; (c) measuring and/or observing one or more electrophysiological metrics of the one or more neuronal cells and/or one or more tissue explants; and (d) correlating one or more electrophysiological metrics of the one or more neuronal cells and/or one or more tissue explants with the toxicity of the agent, such that, if the electrophysiological metrics are indicative of decreased cell viability, the agent is characterized as toxic and, if the electrophysiological metrics are indicative of unchanged or increased cell viability, the agent is characterized as non-toxic; wherein step (c) optionally comprises and/or observing one or more morphometric changes of the one or more neuronal cells and/or one or more tissue explants; and wherein step (d) optionally comprises correlating one or more morphometric changes of the one or more neuronal cells and/or tissue explants with the toxicity of the agent, such that, if the morphometric changes are indicative of decreased cell viability, the agent is characterized as toxic and, if the morphometric changes are indicative of unchanged or increased cell viability, the agent is characterized as non-toxic.

In some embodiments, the at least one agent comprises a small chemical compound. In some embodiments, the at least one agent comprises at least one environmental or industrial pollutant. In some embodiments, the at least one agent comprises one or a combination of small chemical compounds chosen from: chemotherapeutics, analgesics, cardiovascular modulators, cholesterol level modulators, neuroprotectants, neuromodulators, immunomodulators, anti-inflammatories, and anti-microbial drugs such as bacterial antibiotics. In some embodiments, the at least one agent comprises a therapeutically effective amount of an antibody, such as a clinically relevant monoclonal antibody like Tysabri.

In some embodiments, the one or more electrophysiological metrics are one or a combination of: electrical conduction velocity, action potential, amplitude of the wave associated with passage of an electrical impulse along a membrane of one or a plurality of neuronal cells, a width of an electrical impulses along a membrane of one or a plurality of neuronal cells, latency of the electrical impulse along a membrane of one or a plurality of neuronal cells, and envelope of the electrical impulse along a membrane of one or a plurality of neuronal cells. In some embodiments, the one or more electrophysiological metrics comprise compound action potential across a tissue explant.

The present disclosure also relates to method of measuring the amount or degree of myelination or demyelination of one or more axons of one or a plurality of neuronal cells and/or one or a plurality of tissue explants, said method comprising: (a) culturing one or more neuronal cells and/or one or a plurality of tissue explants in any of the compositions disclosed herein for a time and under conditions sufficient to grow at least one axon; (b) measuring and/or observing one or more morphometric changes of the one or more neuronal cells and/or one or more tissue explants; and (c) correlating one or more morphometric changes of the one or more neuronal and/or one or more tissue explants cells with a quantitative or qualitative change of myelination of the neuronal cells or tissue explants.

The present disclosure also relates to a method of measuring myelination or demyelination of one or more axons of one or a plurality of neuronal cells and/or one or a plurality of tissue explants, said method comprising: (a) culturing one or more neuronal cells and/or one or a plurality of tissue explants in any of the compositions disclosed herein for a time and under conditions sufficient to grow at least one axon; (b) measuring and/or observing one or more electrophysiological metrics of the one or more neuronal cells and/or one or more tissue explants; and (c) correlating one or more electrophysiological metrics of the one or more neuronal and/or one or more tissue explants cells with a quantitative or qualitative change of myelination of the neuronal cells or tissue explants; wherein step (b) optionally comprises and/or observing one or more morphometric changes of the one or more neuronal cells and/or one or more tissue explants; and wherein step (c) optionally comprises correlating one or more morphometric changes of the one or more neuronal cells and/or tissue explants with the quantitative or qualitative change of myelination of the neuronal cells or tissue explants.

The present disclosure also relates to a method of measuring myelination or demyelination of one or more axons of one or a plurality of neuronal cells and/or one or a plurality of tissue explants, said method comprising: (a) culturing one or more neuronal cells and/or one or a plurality of tissue explants in any of the compositions disclosed herein for a time and under conditions sufficient to grow at least one axon; and (b) detecting the amount of myelination on one or a plurality of axons of the one or more neuronal cells and/or one or more tissue explants.

In some embodiments, the step of detecting the amount of myelination on one or a plurality of axons of the one or more neuronal cells and/or one or more tissue explants comprises exposing the cells to an antibody that binds to myelin.

In some embodiments, the method further comprises (i) exposing one or a plurality of neuronal cells and/or one or a plurality of tissue explants to at least one agent after steps (a) and (b); (ii) measuring and/or observing one or more electrophysiological metrics, measuring and/or observing one or more morphometric changes and/or detecting the quantitative amount of myelin from the one or a plurality of neuronal cells and/or one or a plurality of tissue explants; (iii) calculating a change of measurements, observations and/or quantitative amount of myelin from the one or a plurality of neuronal cells and/or the one or a plurality of tissue explants in the presence and absence of the agent; and (iv) correlating the change of measurements, observations and/or quantitative amount of myelin from the one or a plurality of neuronal cells and/or the one or a plurality of tissue explants to the presence or absence of the agent.

In some embodiments, the at least one agent comprises at least one environmental or industrial pollutant. In some embodiments, the at least one agent comprises one or a combination of small chemical compounds chosen from: chemotherapeutics, analgesics, cardiovascular modulators, cholesterol level modulators, neuroprotectants, neuromodulators, immunomodulators, anti-inflammatories, and anti-microbial drugs.

In some embodiments, the one or more electrophysiological metrics are one or a combination of: electrical conduction velocity, action potential, amplitude of the wave associated with passage of an electrical impulse along a membrane of one or a plurality of neuronal cells, a width of an electrical impulses along a membrane of one or a plurality of neuronal cells, latency of the electrical impulse along a membrane of one or a plurality of neuronal cells, and envelope of the electrical impulse along a membrane of one or a plurality of neuronal cells. In some embodiments, wherein the one or more electrophysiological metrics comprise compound action potential across a tissue explant.

The present disclosure also relates to a method of measuring myelination or demyelination of one or more axons of one or a plurality of neuronal cells and/or one or a plurality of tissue explants, said method comprising: (a) culturing one or more neuronal cells and/or one or a plurality of tissue explants in any of the compositions disclosed herein for a time and under conditions sufficient to grow at least one axon; and (b) inducing a compound action potential in such one or more neuronal cells and/or one or more tissue explants; (c) measuring the compound action potential; and (d) quantifying the levels of myelination of such one or more neuronal cells based on the compound action potential. In some embodiments, the method further comprises exposing the one or more neuronal cells and/or one or a plurality of tissue explants to an agent. In some embodiments, the at least one agent comprises at least one environmental or industrial pollutant.

In some embodiments, the at least one agent comprises one or a combination of small chemical compounds chosen from: chematherapeutics, analgesics, cardiovascular modulators, cholesterol level modulators, neuroprotectants, neuromodulators, immunomodulators, anti-inflammatories, and anti-microbial drugs.

In some embodiments, the method further comprises measuring one or a plurality of electrophysiological metrics other than compound action potential chosen from one or a combination of: electrical conduction velocity, individual action potential, amplitude of the wave associated with passage of an electrical impulse along a membrane of one or a plurality of neuronal cells and/or tissue explants, a width of an electrical impulses along a membrane of one or a plurality of neuronal cells and/or tissue explants, latency of the electrical impulse along a membrane of one or a plurality of neuronal cells and/or tissue explants, and envelope of the electrical impulse along a membrane of one or a plurality of neuronal cells and/or tissue explants. In some embodiments, the method further comprises measuring one or more morphometric changes associated with the one or more neuronal cells and/or the one or plurality of tissue explants.

The present disclosure also relates to a method of inducing growth of one or a plurality of neuronal cells in a three dimensional culture vessel comprising a solid substrate, said method comprising: (a) seeding one or a plurality of isolated Schwann cells with the solid substrate; (b) seeding one or a plurality of isolated neuronal cells in suspension or isolated neuronal cells in an explant to the at least one interior chamber; (c) introducing a cell culture medium into the culture vessel with a volume sufficient to cover the at least the cells; wherein the solid substrate comprises a first cell-impenetrable polymer and a first cell-penetrable polymer.

In some embodiments, the method further comprises positioning at least one electrode at either end or both ends of the solid substrate, such that the electrodes can be used to stimulate or record action potentials (APs) and or compound action potentials (cAPs) allowing measurement of AP/cAP propagation.

In some embodiments, the composition further comprises placement of at least one electrode providing means for electrical stimulation, wherein the electrode or electrodes are positioned at or distal to the soma of the DRG neurons such that the electrodes create a voltage difference between two points of the neurites/axons to evoke a propogating AP/cAP.

The present disclosure also relates to a method of assessing the response of the neuronal cells in the culture vessel following introduction of one or more stimuli to the one or more neuronal cells; and measuring AP or cAP responses from the one or more neuronal cells to the one or more stimuli using local field potential (LFP) or other single-cell recording methods.

In some embodiments, the solid substrate comprises an exterior surface and an interior surface, such solid substrate comprising at least one portion in a cylindrical or substantially cylindrical shape and at least one hollow interior defined at its edge by at least one portion of the interior surface; said interior surface comprising one or a plurality of pores from about 0.1 microns to about 1.0 microns in diameter, wherein the hollow interior of the solid substrate is accessible from a point exterior to the solid substrate through at least one opening; wherein the hollow interior portion comprises a first portion proximate to the opening and at least a second portion distal to the opening; wherein the one or plurality of neuronal cells and/or the one or plurality of tissue explants are positioned at or proximate to the first portion of the hollow interior and are in physical contact with at last one of the first cell-impenetrable polymer or the first cell-penetrable polymer, and wherein the second portion of the at least on hollow interior is in fluid communication with the first portion such that axons are capable of growth from the one or plurality of neuronal cells and/or the one or plurality of tissue explants into the second interior portion of the hollow interior.

In some embodiments, the method further comprises contacting the one or plurality of neuronal cells with at least one agent. In some embodiments, the at least one agent is one or a plurality of stem cells or modified T cells. In some embodiments, the modified T cells express chimeric antigen receptors specific for a cancer cell. In some embodiments, the cell culture medium comprises one or a combination of: laminin, insulin, transferrin, selenium, BSA, FBS, ascorbic acid, type I collagen, and type III collagen.

The present disclosure also relates to a method of detecting and/or quantifying neuronal cell growth comprising: (a) quantifying one or a plurality of neuronal cells; (b) culturing the one or more neuronal cells in any of the compositions disclosed herein; and (c) calculating the number of neuronal cells in the composition after a culturing for a time period sufficient to allow growth of the one or plurality of cells. In some embodiments, step (c) comprises detecting an internal and/or external recording of such one or more neuronal cells after culturing one or more neuronal cells and correlating the recording with a measurement of the same recording corresponding to a known or control number of cells.

In some embodiments, the method further comprises contacting the one or more neuronal cells to one or more agents. In some embodiments, the method further comprises: (i) measuring an intracellular and/or extracellular recording before and after the step of contacting the one or more neuronal cells to the one or more agents; and (ii) correlating the difference in the recordings before contacting the one or more neuronal cells to the one or more agents to the recording after contacting the one or more neuronal cells to the one or more agents to a change in cell number.

The present disclosure also relates to a method of detecting or quantifying of axon degeneration of one or a plurality of neuronal cells comprising: (a) seeding one or a plurality of neuronal cells in any of the compositions disclosed herein; (b) culturing the one or plurality of neuronal cells for a time period and under conditions sufficient to grow at least one or a plurality of axons from the one or plurality of neuronal cells, (c) quantifying the number or density of axons grown from the neuronal cells; (d) contacting the one or plurality of neuronal cells to one or a plurality of agents; (e) quantifying the number and/or the density of the axons grown from neuronal cells after contacting the one or plurality of cells to one or a plurality of agents; and (f) calculating a difference in the number or density of axons in culture in the presence or absence of the agent.

In some embodiments, the step of the one or plurality of axons and/or the density of the axons grown from neuronal cells comprises staining the one or plurality of a neuronal cells with a dye, fluorophore, or labeled antibody.

In some embodiments, steps (c), (e), and/or (f) are performed via microscopy or digital imaging.

In some embodiments, steps (c) and (e) comprise taking measurements comprises from a portion of one or plurality of axons proximate to one or a plurality soma and taking measurements from a portion of one or plurality of axons distal to one or a plurality soma.

In some embodiments, the difference in the number or density of axons in culture in the presence or absence of the agent is the difference between a portion of the axon or axons proximate to cell bodies of the one or plurality of neuronal cells and a portion of the axons distal from the cell bodies of the one or plurality of neuronal cells.

In some embodiments, taking measurements comprises measuring any one of combination of: morphometric metrics or electrophysiological metrics and wherein the step of calculating a difference in the number or density of axons in culture comprises correlating any one or combination of measurements to the number or density of axons. In some embodiments, taking measurements comprises measuring any one of combination of electrophysiological metrics and wherein the step of calculating a difference in the number or density of axons in culture comprises correlating any one or combination of electrophysiological metrics to the number or density of axons.

In some embodiments, the method further comprises (g) correlating the neurodegenerative effect of an agent to electrophysiological metrics taken in steps (c) and (e).

The present disclosure also relates to method of measuring intracellular or extracellular recordings comprising: (a) culturing one or a plurality of neuronal cells in any of the compositions disclosed herein; (b) applying a voltage potential across the one or a plurality of neuronal cells; and (c) measuring one or a plurality of electrophysiological metrics from the one or a plurality of neuronal cells. In some embodiments, the one or a plurality of electrophysiological metrics other are chosen from one or a combination of: electrical conduction velocity, intracellular action potential, compound action potential, amplitude of the wave associated with passage of an electrical impulse along a membrane of one or a plurality of neuronal cells and/or tissue explants, a width of an electrical impulses along a membrane of one or a plurality of neuronal cells and/or tissue explants, latency of the electrical impulse along a membrane of one or a plurality of neuronal cells and/or tissue explants, and envelope of the electrical impulse along a membrane of one or a plurality of neuronal cells and/or tissue explants.

The present disclosure also relates to a method of measuring or quantifying any neuroprotective effect of an agent comprising: (a) culturing one or a plurality of neuronal cells or tissue explants in any of the compositions disclosed herein in the presence and absence of the agent; (b) applying a voltage potential across the one or a plurality of neuronal cells or tissue explants in the presence and absence of the agent; (c) measuring one or a plurality of electrophysiological metrics from the one or plurality of neuronal cells or tissue explants in the presence and absence of the agent; and (d) correlating the difference in one or a plurality of electrophysiological metrics through the one or plurality of neuronal cells or tissue explants to the neuroprotective effect of the agent, such that a decline in electrophysiological metrics in the presence of the agent as compared to the electrophysiological metrics measured in the absence of the agent is indicative of a poor neuroprotective effect, and no change or an incline of electrophysiological metrics in the presence of the agent as compared to the electrophysiological metrics measured in the absence of the agent is indicative of the agent conferring a neuroprotective effect.

The present disclosure relates to a method of measuring or quantifying any neuromodulatory effect of an agent comprising: (a) culturing one or a plurality of neuronal cells or tissue explants in any of the compositions disclosed herein in the presence and absence of the agent; (b) applying a voltage potential across the one or a plurality of neuronal cells or tissue explants in the presence and absence of the agent; (c) measuring one or a plurality of electrophysiological metrics from the one or plurality of neuronal cells or tissue explants in the presence and absence of the agent; and (d) correlating the difference in one or a plurality of electrophysiological metrics through the one or plurality of neuronal cells or tissue explants to the neuromodulatory effect of the agent, such that a change in electrophysiological metrics in the presence of the agent as compared to the electrophysiological metrics measured in the absence of the agent is indicative of a neuromodulatory effect, and no change of electrophysiological metrics in the presence of the agent as compared to the electrophysiological metrics measured in the absence of the agent is indicative of the agent not conferring a neuromodulatory effect.

The present disclosure also relates to a method of detecting or quantifying myelination or demyelination of an axon in vitro comprising: (a) culturing one or a plurality of neuronal cells in any of the compositions disclosed herein for a time and under conditions sufficient for the one or a plurality of neuronal cells to row one or a plurality of axons; (b) applying a voltage potential across the one or a plurality of neuronal cells; and (c) measuring the field potential or compound action potential through the one or plurality of neuronal cells; (d) calculating the conduction velocity through the one or a plurality of neuronal cells; and (e) correlating the one or plurality of values or conduction velocity with the amount of myelination of one or a plurality of axons.

In some embodiments, the method further comprises correlating the conduction velocity of step (d) to the conduction velocity value of a known or predetermined number of myelinated, healthy neuronal cells.

In some embodiments, the method further comprises exposing the one or a plurality of neuronal cells to an agent; wherein steps (a)-(e) are performed in the presence of the agent and the method further comprises assessing the difference in amounts of myelination due to the presence of the agent in which conduction velocities of the cells in the presence of the agent are compared to conduction velocities of the cells in the absence of the agent.

In some embodiments, the method further comprises imaging the one or plurality of neuronal cells and/or tissue explants with a microscope and/or digital camera.

The present disclosure also relates to a method of culturing a stem cell or immune cell comprising: (a) culturing one or a plurality of neuronal cells and/or tissue explants in any of the compositions disclosed herein; and (b) exposing an isolated stem cell or immune cell to the composition.

The present disclosure also relates to a system comprising: (i) a cell culture vessel comprising a hydrogel; (ii) one or a plurality of neuronal cells either in suspension or as a component of a tissue explant; (iii) an amplifier comprising a generator for electrical current; (iv) a voltmeter and/or ammeter; (v) at least a first stimulating electrode and at least a first recording electrode; wherein the amplifier, voltmeter and/or ammeter, and electrodes are electrically connected to the each other via a circuit in which electrical current is fed to the at least one stimulating electrode from the amplifier and electrical current is received at the recording electrode and fed to the voltmeter and/or ammeter; wherein the stimulating electrode is positioned at or proximate to one or a plurality of soma of the neuronal cells and the recording electrode is positioned at a predetermined distance distal to the soma, such that an electrical field is established across the cell culture vessel.

### BRIEF DESCRIPTION OF THE DRAWINGS

FIGS. 1A-1E depict exemplary micropatterning of PEG constructs with dynamic mask projection photolithography. FIG. 1A depicts an exemplary schematic of digital micromirror device (DMD) dynamic-mask photolithography method. FIG. 1B depicts a macro view of exemplary PEG constructs inside six-well cell culture insert. FIG. 1C depicts a close-up of exemplary PEG constructs inside cell culture insert. FIG. 1D depicts an exemplary DMD photomask. FIG. 1E depicts an exemplary PEG construct crosslinked around adhered DRG.

FIG. 2 depicts the stability of Puramatrix within exemplary PEG constructs relative to volume of PBS added: representative images of fluorescent micrographs of Fluosphere-labeled Puramatrix 48 hours after gelation in PEG (broken white outline indicates PEG void) and schematic diagrams of dual hydrogel constructs are shown above bar plot of Puramatrix stability with respect to volume of PBS added (n=18 for each of three experiments, bars represent standard error of the mean).

FIGS. 3A-3F depict exemplary DRG neurite growth and cell migration in dual hydrogel constructs. FIG. 3A depicts a live/dead stained construct (live cells and cellular structures, dead cells, bright field) after 5 days in culture; FIGS. 3B and 3C depict DRG explants cultured in dual hydrogel constructs for 7 days, indicated by β-III tubulin-positive neurites and DAPI-stained nuclei. FIG. 3D depicts close-up view of leading growth inside channel (β-III tubulin) after 5 days. FIG. 3E depicts a DRG explants cultured for 7 days, stained for MAP2-positive dendrites and β-III tubulin-positive neurites. FIG. 3F depicts a bifurcating portion of the construct focused at the surface of the cell culture insert (β-III tubulin).

FIGS. 4A-4E depict confocal micrographs of β-III tubulin and DAPI (4A only) stained constructs. FIG. 4A depicts a three dimensional representation of growth near bifurcation point, showing both an orthographic view and a side view to demonstrate thickness. Image slices were interpolated to account for distance between slices. FIG. 4B depicts a merged z-stack projection of neurite growth in dual hydrogel construct. FIG. 4C depicts a merged z-stack projection of neurite growth in PEG construct without Puramatrix. FIG. 4D depicts a depth-coded z-stack projection of neurite growth in PEG construct without Puramatrix. FIG. 4E depicts a depth-coded z-stack projection of neurite growth in dual hydrogel construct. In FIGS. 4B-4E, a standard deviation projection was used.

FIGS. 5A-5D depicts fluorescence microscopy of DRG neurite growth and cell migration in three dimensional dual hydrogel constructs after 7 days in vitro: β-III tubulin-positive neurites, DAPI-stained nuclei, and S100-positive glial cells confined within channel filled with Puramatrix; supportive cells present near the end of the channel, approximately 1.875 mm from the ganglion, as measured from the end of the circular region containing ganglion and the start of the straight channel (C-D).

FIGS. 6A-6C depicts three-dimensional rendering of confocal images. β-III tubulin-positive neurites, DAPI-stained nuclei, and S100-positive glial cells shown in 3D at beginning (FIG. 6A), middle (FIG. 6B), and end (FIG. 6C) of channel with corresponding cross-sections in the z-plane shown below.

FIGS. 7A-7D depict transmission electron microscopy of neural culture cross-sections. FIG. 7A depicts high density of parallel, fasciculated unmyelinated neurites in channel approximately 1.875 mm from ganglion, with FIG. 7B inset showing zoomed view. FIG. 7C depicts a focus centered on an axon (Ax) encapsulated by a Schwann cell (SC) approximately 1 mm from the ganglion. FIG. 7D depicts a Schwann cell nucleus (SN) found in ganglion; all measurements made from the end of the circular region containing ganglion at the start of the straight channel.

FIG. 8A depicts a Bromophenol Blue-stained construct with placement of recording (left) and stimulating (right) electrodes placed within ganglion and neural tract in channel, respectively, for field recording. FIG. 8B depicts an example trace of population response demonstrating successful field potential recordings in three dimensional neural constructs and waveform properties characteristic of a compound action potential (CAP). FIG. 8C depicts a field potential evoked in ganglion of three dimensional neural cultures from proximal (1.5 mm) and distal (2.25 mm) locations, n=4; marked by dotted lines. Average traces highlight the increase in delay to onset when stimulating distally. FIG. 8D depicts the distal stimulation caused a significant increase (p=0.02) in delays to onset with average delay time increasing from 0.82 ms to 2.88 ms, as well as a decrease in average response amplitude by 29.46%. Stimulation distances were measured from the start of the straight channel to the point of stimulation. Delay of onset was measured as the time between the return of the stimulus artifact to baseline to the positive peak of the response. FIG. 8E depicts a blockade of Na+ channel activity using 0.5 μM tetrodotoxin (TTX) in three-dimensional neural constructs. Average traces demonstrating abolishment of population response by TTX, n=3. FIG. 8F depicts the response amplitudes were significantly different, p=0.029, with average amplitudes decreasing from 448.75 μV to 0.04 μV after TTX wash-in. Amplitudes were measured from peak-to-peak.

FIG. 9A depicts no effect of excitatory glutamate blockers DNQX and APV in 3D neural constructs, n=4. Average traces of responses before (t1-t5) and after (t16-t20) drug wash-in demonstrate no marked change in response amplitude (FIG. 9B) or duration (FIG. 9C) from drugs. Response amplitudes and durations were of no statistical difference after DNQX and APV. Amplitudes were measured peak-to-peak and durations at half-peak to minimize variance between measurements. FIG. 9D depicts the consistent firing of response during high frequency stimulation in three-dimensional neural constructs, n=3. Example traces demonstrate the consistency of the electrically evoked population spike during the 50 Hz train, with enlarged traces at the start and end for comparison. The amplitudes (FIG. 9E) and duration (FIG. 9F) of responses at the end of the 50 Hz pulse train are not significantly different than those at the start. Amplitudes were measured from peak-to-peak and durations at half-peak to minimize variance between measurements.

FIGS. 10A-10F depict electrophysiological experiments on cultured neurons. FIG. 10A depicts the placement of recording (left) and stimulating (right) electrodes for whole-cell patch clamp. FIG. 10B depicts the successful whole-cell patch clamp of primary sensory neuron in 3D neural constructs. FIG. 10C depicts the successful whole-cell patch clamp recordings in 3D neural cultures exhibiting no evidence of synaptic activity, n=3. Example trace displaying an electrically evoked action potential recorded from a cell in the ganglion. FIG. 10D depicts enlarged trace demonstrating quick, non-graded onset of response. FIG. 10E depicts a voltage clamp trace with no spontaneous currents. FIG. 10F depicts a current clamp trace exhibiting no spontaneous changes in potential.

FIGS. 11A-11B depict an analysis of depth of neurite growth in constructs. FIG. 11A depicts the average height of β-III labeled neurites in constructs both with and without Puramatrix (p<0.005). FIG. 11B depicts neurite growth throughout depth of Puramatrix as a percentage of total neurite growth.

FIGS. 12A-12F depict fluorescent microscopy of neurite growth after 7 days in vitro. FIG. 12A depicts branching and random orientation of leading neurite growth in Puramatrix shown from top focal plane. FIG. 12B depicts branching and random orientation of leading neurite growth in Puramatrix shown from the bottom focal plane. FIG. 12C depicts limited neurite growth along surface of insert membrane in channel without Puramatrix. FIG. 12D depicts preferential growth along PEG boundary. FIG. 12E depicts absence of myelin before Fluoromyelin™ Red Fluorescent Myelin Stain. FIG. 12F depicts absence of myelin after Fluoromyelin™ Red Fluorescent Myelin Stain.

FIG. 13 depicts the methodology for co-culturing SCs and DRGs. Step 1 is formation of PEG mold; Step 2 is DRG insertion; Step 3 is mixing SCs with the gel solution at a specific cell count and addition of the gel solution to the void; Step 4 is irradiation using the negative mask and gel formation.

FIG. 14 depicts the quantification of the amount of neuronal growth in each of the four culture models in three dimensions. More neuronal growth in the two systems with collagen was observed. No significant impact was detected on neuronal outgrowth due to the change in media regimen.

FIG. 15 depicts the development of myelin protein (MBP) after 25 days. DRGs/SCs were co-cultured with neurons fixed and immunolabeled with anti-MBP and beta-III tubulin antibodies for compact myelin and neurofilaments; objective 20×; scale bar represents 25 μm. SCs completely envelop axons after 25 days, forming MBP-positive axons in all experimental groups.

FIGS. 16A-16B depict three-dimensional renderings of confocal images. FIG. 16A depicts the immunohistochemistry for MBP protein. FIG. 16B depicts the immunohistochemistry for MAG. The culture thickness for both is 190 μm, confirming three dimensional myelin formation ability of the in vitro system.

FIGS. 17A-17C depict the immunohistochemistry for neurofilaments β-III and MBP. FIG. 17A visually depicts the immunohistochemistry in various media. The FIGs. are acquired using z-stack acquisition with confocal microscopy. A maximum projection was obtained subsequently. A dense fasciculated growth can be observed after 25 days. Scale bar=500 μm. FIG. 17B depicts a graph of the volume of myelination. The amount of MBP-positive myelin increased in the presence of collagen. NCol-15 with lesser AA exposure has the least amount of myelin. FIG. 17C depicts a graph of the ratio of the volume of MBP-positive myelin to the volume of neurofilaments depicts that cultures with longer exposure to AA form more compact myelin. In all experimental groups, the percentage of myelin formation drastically decreases in the control groups, demonstrating that the exogenic SCs have a major role in myelination process.

FIGS. 18A-18C depict the immunohistochemistry for neurofilaments β-III and PO. FIG. 18A visually depicts the immunohistochemistry in various media. Scale bar=500 fill. FIG. 18B depicts a graph of the volume of myelination. The amount of PO-positive myelin increased in the presence of collagen. PO exists in the PNS compact myelin and therefore PO positive myelin represents the PNS compact myelin. Col-25 with higher AA exposure and incorporation of collagen has the most amount of compact myelin. The decreasing trend shows that removing both factors, the collagen existence and the longer exposure to AA, will result in the least myelin formation in the 3D cultures after 25 days. FIG. 18C depicts a graph of the percentage of PO positive myelin to neurofilaments shows the productivity of the system only in myelin formation despite the volume of neuronal production. Excluding the volume of the neuronal growth shows in the presence or absence of collagen (Col or N-Col), the exposure to AA plays an important role in myelin formation in 3D. However, Col-15 is statistically equivalent with NCol-25, showing that the efficiency of the constructs after 25 days of AA exposure in absence of collagen is similar to that after 15 days of AA exposure in the presence of collagen. Note that the amounts are substantially different as shown in FIG. 18B.

FIGS. 19A-19C depict the immunohistochemistry for neurofilaments β-III and MAG. FIG. 19A visually depicts the immunohistochemistry in various media. MAG is one of the main proteins that is present in the non-compact myelin. Scale bar=500 μm. FIG. 19B depicts a graph of the volume of compact myelin in all four experimental groups. Col-25 with higher AA exposure and incorporation of collagen has the most amount of non-compact myelin. FIG. 19C depicts a graph of the ratio of the volume of MAG-positive myelin to neurofilaments shows that NCol-15 with the shortest time of AA exposure and in the absence of collagen has the least efficiency in non-compact myelin formation, regardless of the volume of nerve fibers in the system.

FIGS. 20A-20F depict transmission electron microscopy pictures of neural culture cross-sections demonstrating myelin sheaths around individual nerve fibers in 25 day cultures: (FIG. 20A) NCol-25; (FIG. 20B) NCol-15; (FIG. 20D) Col-25; (FIG. 20E) Col-15. FIG. 20C depicts a high density of parallel, fasciculated neurites in channel. Neurons are either myelinated or the SCs have started to sheath around the nerve fibers, explaining the high amounts of myelin protein positive in immunohistochemistry staining. FIG. 20F depicts an enlargement of a thick myelin sheath. A=Axons, M=Myelin, S=Schwann cells.

FIGS. 21A-21B depict structure-function correlations. FIG. 21A depicts confocal image stacks of unmyelinated neural fiber tracts proximal to the dorsal root ganglion, a the midpoint, and distal from the ganglion, stained with β-III Tubulin neurites. DAPI nuclei, and S100 Schwann cells. FIG. 21B depicts data showing that recorded cAPs stimulated proximally show higher amplitude and shorter latency than those stimulated distally.

FIGS. 22A-22C depict the physiological evaluation of the neural culture under toxic stress with high glucose conditions. FIG. 21A depicts electrophysiological traces of the cell culture in the presence of 25 mM and 60 mM glucose for 48 hours. FIG. 21B depicts a graph showing that exposure to the 60 mM glucose condition leads to a reduction in compound action potential amplitude. FIG. 21B depicts a graph showing that exposure to the 60 mM glucose condition leads to an increase in compound action potential latency.

FIGS. 23A-23C depict the physiological evaluation of the neural culture under toxic stress with 0.1 μM Paclitaxel. FIG. 22A depicts electrophysiological traces of the cell culture before and after the application of paclitaxel. FIG. 22B depicts a graph showing that exposure to paclitaxel decreases compound action potential amplitude. FIG. 22C depicts a graph showing that exposure to paclitaxel increases compound action potential latency.

FIG. 24 depicts a list of the morphological and physiological measurements that can be taken at the ganglion, at the proximal tract, at the midpoint of the tract, and at the distal tract of a dorsal root ganglion.

FIG. 25 depicts a list of the proposed targets of chemotherapy-induced peripheral neurotoxicity at the dorsal root ganglion, microtubules, ion channels, myelin, mitochondria, and the small nerve fibers.

FIG. 26 depicts an experimental design where baseline physiological recordings will be taken after growth and myelination in culture. Experiments will be limited to an acute (48 hr) application of each drug followed by an immediate or delayed (7 days) assessment by physiological recording (Rec) and imaging (CFM and TEM). The control group will consist of vehicle administration, without drugs.

FIGS. 27A-27B depict a culture of retinal (CNS) tissue. Retinal explants from embryonic rats were cultured within 3D micropatterned hydrogels in “neurobasal Sato” medium supplemented with either CNTF (FIG. 23A) or BDNF (FIG. 23B). Observable retinal ganglion cell axon extension was visualized after one week in culture, stained with β-III tubulin.

FIG. 28 depicts an experiment showing that that DRG neurites grow preferentially toward NGF, as opposed to BSA, diffusing from a reservoir in the hydrogel construct.

FIG. 29 depicts a microphysiological culture systems and noninvasive electro-physiological analyses featuring selective illumination and simultaneous activation of individual cortical neurons as well as individual dendrites in cells expressing GFP and ChR2. This application of DLP microscopy and optogenetics for optical neuroactivation is combined with a voltage-sensitive dye imaging, such as VF.

FIG. 30 depicts a multi-well format utilizing fluorescence microscopy and electrophysiology.

### DETAILED DESCRIPTION

Various terms relating to the methods and other aspects of the present disclosure are used throughout the specification and claims. Such terms are to be given their ordinary meaning in the art unless otherwise indicated. Other specifically defined terms are to be construed in a manner consistent with the definition provided herein.

As used in this specification and the appended claims, the singular forms “a,” “an,” and “the” include plural referents unless the content clearly dictates otherwise.

The term “more than 2” as used herein is defined as any whole integer greater than the number two, e.g. 3, 4, or 5.

The term “plurality” as used herein is defined as any amount or number greater or more than 1.

The term “bioreactor” refers to an enclosure or partial enclosure in which cells are cultured, optionally in suspension. In some embodiments, the bioreactor refers to an enclosure or partial enclosure in which cells are cultured where said cells may be in liquid suspension, or alternatively may be growing in contact with, on, or within another non-liquid substrate including but not limited to a solid growth support material. In some embodiments, the solid growth support material, or solid substrate, comprises at least one or a combination of: silica, plastic, metal, hydrocarbon, or gel. The disclosure relates to a system comprising a bioreactor comprising one or a plurality of culture vessels into which neuronal cells may be cultured in the presence or cellular growth media.

The term “culture vessel” as used herein is defined as any vessel suitable for growing, culturing, cultivating, proliferating, propagating, or otherwise similarly manipulating cells. A culture vessel may also be referred to herein as a “culture insert”. In some embodiments, the culture vessel is made out of biocompatible plastic and/or glass. In some embodiments, the plastic is a thin layer of plastic comprising one or a plurality of pores that allow diffusion of protein, nucleic acid, nutrients (such as heavy metals and hormones) antibiotics, and other cell culture medium components through the pores. in some embodiments, the pores are not more than about 0.1, 0.5 1.0, 2, 3, 4, 5, 6, 7, 8, 9, 10, 15, 20, 25, 30, 40, 50 microns wide. In some embodiments, the culture vessel in a hydrogel matrix and free of a base or any other structure. In some embodiments, the culture vessel is designed to contain a hydrogel or hydrogel matrix and various culture mediums. In some embodiments, the culture vessel consists of or consists essentially of a hydrogel or hydrogel matrix. In some embodiments, the only plastic component of the culture vessel is the components of the culture vessel that make up the side walls and/or bottom of the culture vessel that separate the volume of a well or zone of cellular growth from a point exterior to the culture vessel. In some embodiments, the culture vessel comprises a hydrogel and one or a plurality of isolated glial cells. In some embodiments, the culture vessel comprises a hydrogel and one or a plurality of isolated glial cells, to which one or a plurality of neuronal cells are seeded.

The term “electrical stimulation” refers to a process in which the cells are being exposed to an electrical current of either alternating current (AC) or direct current (DC). The current may be introduced into the solid substrate or applied via the cell culture media or other suitable components of the cell culture system. In some embodiments, the electrical stimulation is provided to the device or system by positioning one or a plurality of electrodes at different positions within the device or system to create a voltage potential across the cell culture vessel. The electrodes are in operable connection with one or a plurality of amplifiers, voltmeters, ammeters, and/or electrochemical systems (such as batteries or electrical generators) by one or a plurality of wires. Such devices and wires create a circuit through which an electrical current is produced and by which an electrical potential is produced across the tissue culture system.

The term “hydrogel” as used herein is defined as any water-insoluble, crosslinked, three-dimensional network of polymer chains with the voids between polymer chains filled with or capable of being filled with water. The term “hydrogel matrix” as used herein is defined as any three-dimensional hydrogel construct, system, device, or similar structure. Hydrogels and hydrogel matrices are known in the art and various types have been described, for example, in U.S. Pat. Nos. 5,700,289, and 6,129,761; and in Curley and Moore, 2011; Curley et al., 2011; Irons et al., 2008; and Tibbitt and Anseth, 2009; each of which are incorporated by reference in their entireties. In some embodiments, the hydrogel or hydrogel matrix can be solidified by subjecting the liquefied pregel solution to ultraviolet light, visible light or ay light above about 300 nm, 400 nm, 450 nm or 500 nm in wavelength. In some embodiments, the hydrogel or hydrogel matrix can be solidified into various shapes, for example, a bifurcating shape designed to mimic a neuronal tract. In some embodiments, the hydrogel or hydrogel matrix comprises poly (ethylene glycol) dimethacrylate (PEG). In some embodiments, the hydrogel or hydrogel matrix comprises Puramatrix. In some embodiments, the hydrogel or hydrogel matrix comprises glycidyl methacrylate-dextran (MeDex). In some embodiments, neuronal cells are incorporated in the hydrogel or hydrogel matrices. In some embodiments, cells from nervous system are incorporated into the hydrogel or hydrogel matrices. In some embodiments, the cells from nervous system are Schwann cells and/or oligodendrocytes. In some embodiments, the hydrogel or hydrogel matrix comprises tissue explants from the nervous system of an animal, (such as a mammal) and a supplemental population of cells derived from the nervous system but isolated and cultured to enrich its population in the culture. In some embodiments, the hydrogel or hydrogel matrix comprises a tissue explant such as a retinal tissue explant, DRG, or spinal cord tissue explant and a population of isolated and cultured Schwann cells, oligodendrocytes, and/or microglial cells. In some embodiments, two or more hydrogels or hydrogel matrixes are used simultaneously cell culture vessel. In some embodiments, two or more hydrogels or hydrogel matrixes are used simultaneously in the same cell culture vessel but the hydrogels are separated by a wall that create independently addressable microenvironments in the tissue culture vessel such as wells. In a multiplexed tissue culture vessel it is possible for some embodiments to include any number of aforementioned wells or independently addressable location within the cell culture vessel such that a hydrogel matrix in one well or location is different or the same as the hydrogel matrix in another well or location of the cell culture vessel.

In some embodiments, the two or more hydrogels may comprise different amount of PEG and/or Puramatrix. In some embodiments, the two or more hydrogels may have various densities. In some embodiments, the two or more hydrogels may have various permeabilities that are capable of allowing cells to grow within the hydrogel. In some embodiments, the two or more hydrogels may have various flexibilities.

The term “cell-penetrable polymer” refers to a hydrophilic polymer, with identical or mixed monomer subunits, at a concentration and/or density sufficient to create spaces upon crosslinking in a solid or semisolid state on a solid substrate, such space are sufficiently biocompatible such that a cell or part of a cell can grow in culture.

The term “cell-impenetrable polymer” refers to a hydrophilic polymer, with identical or mixed monomer subunits, at a concentration and/or density sufficient to, upon crosslinking in a solid or semisolid state on a solid substrate, not create biocompatible spaces or compartments. In other words, an cell-impenetrable polymer is a polymer that, after crosslinking at a particular concentration and/or density, cannot support growth of a cell or part of a cell in culture.

One of ordinary skill can appreciate that a cell-impenetrable polymer and a cell-penetrable polymer may comprise the same or substantially the same polymers but the difference in concentration or density after crosslinking creates a hydrogel matrix with some portions conducive to grow a cell or part of cell in culture.

In some embodiments, the hydrogel or hydrogel matrixes can have various thicknesses. In some embodiments, the thickness of the hydrogel or hydrogel matrix is from about 100 μm to about 800 μm. In some embodiments, the thickness of the hydrogel or hydrogel matrix is from about 150 μm to about 800 μm. In some embodiments, the thickness of the hydrogel or hydrogel matrix is from about 200 μm to about 800 μm. In some embodiments, the thickness of the hydrogel or hydrogel matrix is from about 250 μm to about 800 μm. In some embodiments, the thickness of the hydrogel or hydrogel matrix is from about 300 μm to about 800 μm. In some embodiments, the thickness of the hydrogel or hydrogel matrix is from about 350 μm to about 800 μm. In some embodiments, the thickness of the hydrogel or hydrogel matrix is from about 400 μm to about 800 μm. In some embodiments, the thickness of the hydrogel or hydrogel matrix is from about 450 μm to about 800 μm. In some embodiments, the thickness of the hydrogel or hydrogel matrix is from about 500 μm to about 800 μm. In some embodiments, the thickness of the hydrogel or hydrogel matrix is from about 550 μm to about 800 μm. In some embodiments, the thickness of the hydrogel or hydrogel matrix is from about 600 μm to about 800 μm. In some embodiments, the thickness of the hydrogel or hydrogel matrix is from about 650 μm to about 800 μm. In some embodiments, the thickness of the hydrogel or hydrogel matrix is from about 700 μm to about 800 μm. In some embodiments, the thickness of the hydrogel or hydrogel matrix is from about 750 μm to about 800 μm. In some embodiments, the thickness of the hydrogel or hydrogel matrix is from about 100 μm to about 750 μm. In some embodiments, the thickness of the hydrogel or hydrogel matrix is from about 100 μm to about 700 μm. In some embodiments, the thickness of the hydrogel or hydrogel matrix is from about 100 μm to about 650 μm. In some embodiments, the thickness of the hydrogel or hydrogel matrix is from about 100 μm to about 600 μm. In some embodiments, the thickness of the hydrogel or hydrogel matrix is from about 100 μm to about 550 μm. In some embodiments, the thickness of the hydrogel or hydrogel matrix is from about 100 μm to about 500 μm. In some embodiments, the thickness of the hydrogel or hydrogel matrix is from about 100 μm to about 450 μm. In some embodiments, the thickness of the hydrogel or hydrogel matrix is from about 100 μm to about 400 μm. In some embodiments, the thickness of the hydrogel or hydrogel matrix is from about 100 μm to about 350 μm. In some embodiments, the thickness of the hydrogel or hydrogel matrix is from about 100 μm to about 300 μm. In some embodiments, the thickness of the hydrogel or hydrogel matrix is from about 100 μm to about 250 μm. In some embodiments, the thickness of the hydrogel or hydrogel matrix is from about 100 μm to about 200 μm. In some embodiments, the thickness of the hydrogel or hydrogel matrix is from about 100 μm to about 150 μm. In some embodiments, the thickness of the hydrogel or hydrogel matrix is from about 300 μm to about 600 μm. In some embodiments, the thickness of the hydrogel or hydrogel matrix is from about 400 μm to about 500 μm.

In some embodiments, the hydrogel or hydrogel matrixes can have various thicknesses. In some embodiments, the thickness of the hydrogel or hydrogel matrix is from about 10 μm to about 3000 μm. In some embodiments, the thickness of the hydrogel or hydrogel matrix is from about 150 μm to about 3000 μm. In some embodiments, the thickness of the hydrogel or hydrogel matrix is from about 200 μm to about 3000 μm. In some embodiments, the thickness of the hydrogel or hydrogel matrix is from about 250 μm to about 3000 μm. In some embodiments, the thickness of the hydrogel or hydrogel matrix is from about 300 μm to about 3000 μm. In some embodiments, the thickness of the hydrogel or hydrogel matrix is from about 350 μm to about 3000 μm. In some embodiments, the thickness of the hydrogel or hydrogel matrix is from about 400 μm to about 3000 μm. In some embodiments, the thickness of the hydrogel or hydrogel matrix is from about 450 μm to about 3000 μm. In some embodiments, the thickness of the hydrogel or hydrogel matrix is from about 500 μm to about 3000 μm. In some embodiments, the thickness of the hydrogel or hydrogel matrix is from about 550 μm to about 3000 μm. In some embodiments, the thickness of the hydrogel or hydrogel matrix is from about 600 μm to about 3000 μm. In some embodiments, the thickness of the hydrogel or hydrogel matrix is from about 650 μm to about 3000 μm. In some embodiments, the thickness of the hydrogel or hydrogel matrix is from about 700 μm to about 3000 μm. In some embodiments, the thickness of the hydrogel or hydrogel matrix is from about 750 μm to about 3000 μm. In some embodiments, the thickness of the hydrogel or hydrogel matrix is from about 800 μm to about 3000 μm. In some embodiments, the thickness of the hydrogel or hydrogel matrix is from about 850 μm to about 3000 μm. In some embodiments, the thickness of the hydrogel or hydrogel matrix is from about 900 μm to about 3000 μm. In some embodiments, the thickness of the hydrogel or hydrogel matrix is from about 950 μm to about 3000 μm. In some embodiments, the thickness of the hydrogel or hydrogel matrix is from about 1000 μm to about 3000 μm. In some embodiments, the thickness of the hydrogel or hydrogel matrix is from about 1500 μm to about 3000 μm. In some embodiments, the thickness of the hydrogel or hydrogel matrix is from about 2000 μm to about 3000 μm. In some embodiments, the thickness of the hydrogel or hydrogel matrix is from about 2500 μm to about 3000 μm. In some embodiments, the thickness of the hydrogel or hydrogel matrix is from about 100 μm to about 2500 μm. In some embodiments, the thickness of the hydrogel or hydrogel matrix is from about 100 μm to about 2000 μm. In some embodiments, the thickness of the hydrogel or hydrogel matrix is from about 100 μm to about 1500 μm. In some embodiments, the thickness of the hydrogel or hydrogel matrix is from about 100 μm to about 1000 μm. In some embodiments, the thickness of the hydrogel or hydrogel matrix is from about 100 μm to about 950 μm. In some embodiments, the thickness of the hydrogel or hydrogel matrix is from about 100 μm to about 900 μm. In some embodiments, the thickness of the hydrogel or hydrogel matrix is from about 100 μm to about 850 μm. In some embodiments, the thickness of the hydrogel or hydrogel matrix is from about 100 μm to about 800 μm. In some embodiments, the thickness of the hydrogel or hydrogel matrix is from about 100 μm to about 750 μm. In some embodiments, the thickness of the hydrogel or hydrogel matrix is from about 100 μm to about 700 μm. In some embodiments, the thickness of the hydrogel or hydrogel matrix is from about 100 μm to about 650 μm. In some embodiments, the thickness of the hydrogel or hydrogel matrix is from about 100 μm to about 600 μm. In some embodiments, the thickness of the hydrogel or hydrogel matrix is from about 100 μm to about 550 μm. In some embodiments, the thickness of the hydrogel or hydrogel matrix is from about 100 μm to about 500 μm. In some embodiments, the thickness of the hydrogel or hydrogel matrix is from about 100 μm to about 450 μm. In some embodiments, the thickness of the hydrogel or hydrogel matrix is from about 100 μm to about 400 μm. In some embodiments, the thickness of the hydrogel or hydrogel matrix is from about 100 μm to about 350 μm. In some embodiments, the thickness of the hydrogel or hydrogel matrix is from about 100 μm to about 300 μm. In some embodiments, the thickness of the hydrogel or hydrogel matrix is from about 100 μm to about 250 μm. In some embodiments, the thickness of the hydrogel or hydrogel matrix is from about 100 μm to about 200 μm. In some embodiments, the thickness of the hydrogel or hydrogel matrix is from about 100 μm to about 150 μm. In some embodiments, the thickness of the hydrogel or hydrogel matrix is from about 300 μm to about 600 μm. In some embodiments, the thickness of the hydrogel or hydrogel matrix is from about 400 μm to about 500 μm.

In some embodiments, the hydrogel or hydrogel matrix comprises one or more synthetic polymers. In some embodiments, the hydrogel or hydrogel matrix comprises one or more of the following synthetic polymers: polyethylene glycol (polyethylene oxide), polyvinyl alcohol, poly-2-hydroxyethyl methacrylate, polyacrylamide, silicones, and any derivatives or combinations thereof.

In some embodiments, the hydrogel or hydrogel matrix comprises one or more synthetic and/or natural polysaccharides. In some embodiments, the hydrogel or hydrogel matrix comprises one or more of the following polysaccharides: hyaluronic acid, heparin sulfate, heparin, dextran, agarose, chitosan, alginate, and any derivatives or combinations thereof.

In some embodiments, the hydrogel or hydrogel matrix comprises one or more proteins and/or glycoproteins. In some embodiments, the hydrogel or hydrogel matrix comprises one or more of the following proteins: collagen, gelatin, elastin, titin, laminin, fibronectin, fibrin, keratin, silk fibroin, and any derivatives or combinations thereof.

In some embodiments, the hydrogel or hydrogel matrix comprises one or more synthetic and/or natural polypeptides. In some embodiments, the hydrogel or hydrogel matrix comprises one or more of the following polypeptides: polylysine, polyglutamate or polyglycine. In some embodiments, the hydrogel comprises one or a combination of polymers sletec from those published in Khoshakhlagh et al., “Photoreactive interpenetrating network of hyaluronic acid and Puramatrix as a selectively tunable scaffold for neurite growth” Acta Biomaterialia, Jan. 21, 2015.

Any hydrogel suitable for cell growth can be formed by placing any one or combination of polymers disclosed herein at a concentration and under conditions and for a sufficient time period sufficient to create two distinct densities of crosslinked polymers: one cell-penetrable and one cell-impenetrable. The polymers may be synthetic polymers, polysaccharides, natural proteins or glycoproteins and/or polypeptides such as those selected from below.

### Synthetic Polymers

Such as polyethylene glycol (polyethylene oxide), polyvinyl alcohol, poly-2-hydroxyethyl methacrylate, polyacrylamide, silicones, their combinations, and their derivatives.
Polysaccharides (Whether Synthetic or Derived from Natural Sources)
Such as hyaluronic acid, heparan sulfate, heparin, dextran, agarose, chitosan, alginate, their combinations, and their derivatives.

### Natural Proteins or Glycoproteins

Such as collagen, gelatin, elastin, titin, laminin, fibronectin, fibrin, keratin, silk fibroin, their combinations, and their derivatives.

### Polypeptides (Whether Synthetic or Natural Sources)

Such as polylysine, and all of the RAD and EAK peptides already listed.

The term “isolated neurons” refers to neuronal cells that have been removed or disassociated from an organism or culture from which they originally grow. In some embodiments isolated neurons are neurons in suspension. In some embodiments, isolated neurons are a component of a larger mixture of cells including a tissue sample or a suspension with non-neuronal cells. In some embodiments, neuronal cells have become isolated when they are removed from the animal from which they are derived, such as in the case of a tissue explant. In some embodiments isolated neurons are those neurons in a DRG excised from an animal. In some embodiments, the isolated neurons comprise at least one or a plurality cells that are from one species or a combination of the species chosen from: sheep cells, goat cells, horse cells, cow cells, human cells, monkey cells, mouse cells, rat cells, rabbit cells, canine cells, feline cells, porcine cells, or other non-human mammals. In some embodiments, the isolated neurons are human cells. In some embodiments, the isolated neurons are stem cells that are pre-conditioned to have a differentiated phenotype similar to or substantially similar to a human neuronal cell. In some embodiments, the isolated neurons are human cells. In some embodiments, the isolated neurons are stem cells that are pre-conditioned to have a differentiated phenotype similar to or substantially similar to a non-human neuronal cell. In some embodiments, the stem cells are selected from: mesenchymal stem cells, induce pluripotent stem cells, embryonic stem cells, hematopoietic stem cells, epidermal stem cells, stem cells isolated from the umblicial cord of a mammal, or endodermal stem cells.

The term “neurodegenerative disease” is used throughout the specification to describe a disease which is caused by damage to the central nervous system ad or peripheral nervous system. Exemplary neurodegenerative diseases which may be examples of diseases that could be studied using the disclosed model, system or device include for example, Parkinson's disease, Huntington's disease, amyotrophic lateral sclerosis (Lou Gehrig's disease), Alzheimer's disease, lysosomal storage disease (“white matter disease” or glial/demyelination disease, as described, for example by Folkerth, J. Neuropath. Exp. Neuro., 58, 9 Sep. 1999), Tay Sachs disease (beta hexosamimidase deficiency), other genetic diseases, multiple sclerosis, brain injury or trauma caused by ischemia, accidents, environmental insult, etc., spinal cord damage, ataxia and alcoholism. In addition, the present invention may be used to test the efficacy, toxicity, or neurodegenerative effect of agents on neuronal cells in culture for the study of treatments for neurodegenerative diseases. The term neurodegenerative diseases also includes neurodevelopmental disorders including for example, autism and related neurological diseases such as schizophrenia, among numerous others.

The term “neuronal cells” as used herein are defined as cells that comprise at least one or a combination of dendrites, axons, and somata, or, alternatively, any cell or group of cells isolated from nervous system tissue. In some embodiments, neuronal cells are any cell that comprises or is capable of forming an axon. In some embodiments, the neuronal cell is a Schwann cells, glial cell, neuroglia, cortical neuron, embryonic cell isolated from or derived from neuronal tissue or that has differentiated into a cell with a neuronal phenotype or a phenotype which is substantially similar to a phenotype of a neuronal cell, induced pluripotent stem cells (iPS) that have differentiated into a neuronal phenotype, or mesenchymal stem cells that are derived from neuronal tissue or differentiated into a neuronal phenotype. In some embodiments, neuronal cells are neurons from dorsal root gangila (DRG) tissue, retinal tissue, spinal cord tissue, or brain tissue from an adult, adolescent, child or fetal subject. In some embodiments, neuronal cells are any one or plurality of cells isolated from the neuronal tissue of a subject. In some embodiments, the neuronal cells are mammalian cells. In some embodiments, the cells are human cells. In some embodiments, the cells are non-human mammalian cells or derived from cells that are isolated from non-human mammals. If isolated or disassociated from the original animal from which the cells are derived, the neuronal cells may comprises isolated neurons from more than one species.

In some embodiments, neuronal cells are one or more of the following neurons: sympathetic neurons, spinal motor neurons, central nervous system neurons, motor neurons, sensory neurons, cholinergic neurons, GABAergic neurons, glutamatergic neurons, dopaminergic neurons, serotonergic neurons, interneurons, adrenergic neurons, and trigeminal ganglion neurons. In some embodiments, neuronal cells are one or more of the following glial cells: astrocytes, oligodendrocytes, Schwaan cells, microglia, ependymal cells, radial glia, satellite cells, enteric glial cells, and pituyicytes. In some embodiments, neuronal cells are one or more of the following immune cells: macrophages, T cells, B cells, leukocytes, lymphocytes, monocytes, mast cells, neutrophils, natural killer cells, and basophils. In some embodiments, neuronal cells are one or more of the following stem cells: hematopoetic stem cells, neural stem cells, adipose derived stem cells, bone marrow derived stem cells, induced pluripotent stem cells, astrocyte derived induced pluripotent stem cells, fibroblast derived induced pluripotent stem cells, renal epithelial derived induced pluripotent stem cells, keratinocyte derived induced pluripotent stem cells, peripheral blood derived induced pluripotent stem cells, hepatocyte derived induced pluripotent stem cells, mesenchymal derived induced pluripotent stem cells, neural stem cell derived induced pluripotent stem cells, adipose stem cell derived induced pluripotent stem cells, preadipocyte derived induced pluripotent stem cells, chondrocyte derived induced pluripotent stem cells, and skeletal muscle derived induced pluripotent stem cells. In some embodiments, neuronal cells are kartinocytes. In some embodiments, neuronal cells are endothelial cells.

The terms “neuronal cell culture medium” or simply “culture medium” as used herein are defined as any nutritive substance suitable for supporting the growth, culture, cultivating, proliferating, propagating, or otherwise manipulating neuronal cells. In some embodiments, the medium comprises neurobasal medium supplemented with nerve growth factor (NGF). In some embodiments, the medium comprises fetal bovine serum (FBS). In some embodiments, the medium comprises L-glutamine. In some embodiments, the medium comprises ascorbic acid in a concentration ranging from about 0.001% weight by volume to about 0.01% weight by volume. In some embodiments, the medium comprises ascorbic acid in a concentration ranging from about 0.001% weight by volume to about 0.008% weight by volume. In some embodiments, the medium comprises ascorbic acid in a concentration ranging from about 0.001% weight by volume to about 0.006% weight by volume. In some embodiments, the medium comprises ascorbic acid in a concentration ranging from about 0.001% weight by volume to about 0.004% weight by volume. In some embodiments, the medium comprises ascorbic acid in a concentration ranging from about 0.002% weight by volume to about 0.01% weight by volume. In some embodiments, the medium comprises ascorbic acid in a concentration ranging from about 0.003% weight by volume to about 0.01% weight by volume. In some embodiments, the medium comprises ascorbic acid in a concentration ranging from about 0.004% weight by volume to about 0.01% weight by volume. In some embodiments, the medium comprises ascorbic acid in a concentration ranging from about 0.006% weight by volume to about 0.01% weight by volume. In some embodiments, the medium comprises ascorbic acid in a concentration ranging from about 0.008% weight by volume to about 0.01% weight by volume. In some embodiments, the medium comprises ascorbic acid in a concentration ranging from about 0.002% weight by volume to about 0.006% weight by volume. In some embodiments, the medium comprises ascorbic acid in a concentration ranging from about 0.003% weight by volume to about 0.005% weight by volume.

In some embodiments, the hydrogel, hydrogel matrix, and/or neuronal cell culture medium comprises any one or more of the following components: artemin, ascorbic acid, ATP, β-endorphin, BDNF, bovine calf serum, bovine serum albumin, calcitonin gene-related peptide, capsaicin, carrageenan, CCL2, ciliary neurotrophic factor, CX3CL1, CXCL1, CXCL2, D-serine, fetal bovine serum, fluorocitrate. formalin, glial cell line-derived neurotrophic factor, glial fibrillary acid protein, glutamate, IL-1, IL-1α, IL-1β, IL-6, IL-10, IL-12, IL-17, IL-18, insulin, laminin, lipoxins, mac-1-saporin, methionine sulfoximine, minocycline, neuregulin-1, neuroprotectins, neurturin, NGF, nitric oxide, NT-3, NT-4, persephin, platelet lysate, PMX53, Poly-D-lysine (PLL), Poly-L-lysine (PLL), propentofylline, resolvins, S100 calcium-binding protein B, selenium, substance P, TNF-α, type I-V collagen, and zymosan.

As described herein, the term “optogenetics” refers to a biological technique which involves the use of light to control cells in living tissue, typically neurons, that have been genetically modified to express light-sensitive ion channels. It is a neuromodulation method employed in neuroscience that uses a combination of techniques from optics and genetics to control and monitor the activities of individual neurons in living tissue—even within freely-moving animals—and to precisely measure the effects of those manipulations in real-time. The key reagents used in optogenetics are light-sensitive proteins. Spatially-precise neuronal control is achieved using optogenetic actuators like channelrhodopsin, halorhodopsin, and archaerhodopsin, while temporally-precise recordings can be made with the help of optogenetic sensors for calcium (Aequorin, Cameleon, GCaMP), chloride (Clomeleon) or membrane voltage (Mermaid). In some embodiments, neural cells modified with optogenetic actuators and/or sensors are used in the culture systems described herein.

The term “plastic” refers to biocompatible polymers comprising hydrocarbons. In some embodiments, the plastic is selected from the group consisting of: Polystyrene (PS), Poly acrylo nitrile (PAN), Poly carbonate (PC), polyvinylpyrrolidone, polybutadiene (PVP), Polyvinyl butyral (PVB), Poly vinyl chloride (PVC), Poly vinyl methyl ether (PVME), poly lactic-co-glycolic acid (PLGA), poly(1-lactic acid), polyester, polycaprolactone (PCL), poly ethylene oxide (PEO), polyaniline (PANI), polyflourenes, polypyrroles (PPY), poly ethylene dioxythiophene (PEDOT), and a mixture of two or any of the foregoing polymers.

The term “seeding” as used herein is defined as transferring an amount of cells into a new culture vessel. The amount may be defined and may use volume or number of cells as the basis of the defined amount. The cells may be part of a suspension.

The term “solid substrate” as used herein refers to any substance that is a solid support that is free of or substantially free of cellular toxins. In some embodiments, the solid substrate comprise one or a combination of silica, plastic, and metal. In some embodiments, the solid substrate comprises pores of a size and shape sufficient to allow diffusion or non-active transport of proteins, nutrients, and gas through the solid substrate in the presence of a cell culture medium. In some embodiments, the pore size is no more than about 10, 9, 8, 7, 6, 5, 4, 3, 2, 1 micron microns in diameter. One of ordinary skill could determine how big of a pore size is necessary based upon the contents of the cell culture medium and exposure of cells growing on the solid substrate in a particular microenvironment. For instance, one of ordinary skill in the art can observe whether any cultured cells in the system or device are viable under conditions with a solid substrate comprises pores of various diameters. In some embodiments, the solid substrate comprises a base with a predetermined shape that defines the shape of the exterior and interior surface. In some embodiments, the base comprises one or a combination of silica, plastic, ceramic, or metal and wherein the base is in a shape of a cylinder or in a shape substantially similar to a cylinder, such that the first cell-impenetrable polymer and a first cell-penetrable polymer coat the interior surface of the base and define a cylindrical or substantially cylindrical interior chamber; and wherein the opening is positioned at one end of the cylinder. in some embodiments, the base comprises one or a plurality of pores of a size and shape sufficient to allow diffusion of protein, nutrients, and oxygen through the solid substrate in the presence of the cell culture medium. In some embodiments, the solid substrate comprises a plastic base with a pore size of no more than 1 micron in diameter and comprises at least one layer of hydrogel matrix; wherein the hydrogel matrix comprises at least a first cell-impenetrable polymer and at least a first cell-penetrable polymer; the base comprises a predetermined shape around which the first cell-impenetrable polymer and at least a first cell-penetrable polymer physically adhere or chemically bond; wherein the solid substrate comprises at least one compartment defined at least in part by the shape of an interior surface of the solid substrate and accessible from a point outside of the solid substrate by an opening, optionally positioned at one end of the solid substrate. In embodiments, where the solid substrate comprises a hollow interior portion defined by at least one interior surface, the cells in suspension or tissue explants may be seeded by placement of cells at or proximate to the opening such that the cells may adhere to at least a portion the interior surface of the solid substrate for prior to growth. The at least one compartment or hollow interior of the solid substrate allows a containment of the cells in a particular three-dimensional shape defined by the shape of the interior surface solid substrate and encourages directional growth of the cells away from the opening. In the case of neuronal cells, the degree of containment and shape of the at least one compartment are conducive to axon growth from soma positioned within the at least one compartment and at or proximate to the opening. in some embodiments, the solid substrate is tubular or substantially tubular such that the interior compartment is cylindrical or partially cylindrical in shape. In some embodiments, the solid substrate comprises one or a plurality of branched tubular interior compartments. In some embodiments, the bifurcating or multiply bifurcating shape of the hollow interior portion of the solids is configured for or allows axons to grow in multiple branched patterns. When and if electrodes are placed at to near the distal end of an axon and at or proximate to a neuronal cell soma, electrophysiological metrics, such as intracellular action potential can be measured within the device or system.

The disclosure also relates to a system comprising:

(i) a hydrogel matrix;

(ii) one or a plurality of neuronal cells either in suspension or as a component of a tissue explant;

(iii) a generator for electrical current;

(iv) a voltmeter and/or ammeter;

(v) at least a first stimulating electrode and at least a first recording electrode;

wherein the generator, voltmeter and/or ammeter, and electrodes are electrically connected to the each other via a circuit in which electrical current is fed to the at least one stimulating electrode from the generator and electrical current is received at the recording electrode and fed to the voltmeter and/or ammeter; wherein the stimulating electrode is positioned at or proximate to one or a plurality of soma of the neuronal cells and the recording electrode is positioned at a predetermined distance distal to the soma, such that an electrical potential is established across the cell culture vessel.

In some embodiments, the solid substrate consists of hydrogel or hydrogel matrix. In some embodiments, the solid substrate consists of hydrogel or hydrogel matrix and is free of glass, metal, or ceramic. In some embodiments, the solid substrate is shaped into a form or mold that is predetermined for seeding cells of a particular size suitable for axonal growth. In some embodiments, the solid substrate or at least one base portion is shaped with at least one branched interior tube like structure with an optional tapering in diameter the more distal the position of the tube is from the position in which the seeding of the tissue explants or neuronal cells takes place. For instance, this disclosure contemplates a focal point at one end of a semi-cylindrical or cylindrical portion of the solid substrate accessible to a point exterior to the solid substrate by an opening or hole at the exterior surface. The opening or hole can be used to place or seed cells (either neuronal cells and/or glial cells) at the above focal point. As the cells are allowed to grow in culture over several days, the cells are exposed to culture medium with any of the components disclosed herein at concentrations and for a time period sufficient for axons to grow from the neuronal cells. If the cells are to be myelinated or the myelination is desired for study, glial cells may be introduced through the same hole and seeded prior to addition of the neuronal cells or explants. As the axons grow in the semi-cylindrical or tube-like structure, the axonal process growth can occur more and more distal from the focal point. Access points or opening in the solid substrate at points increasingly distal from the focal point (or seding point) can be used to address or observe axonal growth of axon status. This disclosure contemplates the structure of the solid substrate to take any form to encourage axonal growth. In some embodiments, the interior chamber or compartment that houses the axonal process comprises a semi-circular or substantially cylindrical diameter. In some embodiments, the solid substrate is branched in two or more interior compartments at a point distal from the focal point. In some embodiments, this branching can resemble a keyhole shape or tree in which there are 2, 3, 4, 5, 6, 7, or 8 or more tube-like or substantially cylindrical interior chambers in fluid communication with each other such that the axonal growth originates from the seeding point of one or a plurality of somata and extends longitudinally along the interior chamber and into any one or plurality of branches. In some embodiments, one or a plurality of electrodes can be placed at or proximate to one or more openings such that recordings can be taken across one or a plurality of positions along an axon length. This can be used to also interrogate one or multiple positions along the length of the axon.

The term “recording” as used herein is defined as measuring the responses of one or more neuronal cells. Such responses may be electro-physiological responses, for example, patch clamp electrophysiological recordings or field potential recordings.

The present disclosure discloses methods and devices to obtain physiological measurements of a microscale organotypic model of in vitro nerve tissue that mimics clinical nerve conduction and NFD tests. The results obtained from the use of these methods and devices are better predictive of clinical outcomes, enabling a more cost-effective approach for selecting promising lead compounds with higher chances of late-stage success. The disclosure includes the fabrication and utilization of a three-dimensional microengineered system that enables the growth of a uniquely dense, highly parallel neural fiber tract. Due to the confined nature of the tract, this in vitro model is capable of measuring both CAPs and intracellular patch clamp recordings. In addition, subsequent confocal and transmission electron microscopy (TEM) analysis allows for quantitative structural analysis, including NFD. Taken together, the in vitro model system has the novel ability to assess tissue morphometry and population electrophysiology, analogous to clinical histopathology and nerve conduction testing.

The present disclosure also provides a method for measuring the myelination of axons created using the in vitro model described herein. Similar to the structure of a human afferent peripheral nerve, dorsal root ganglion (DRG) neurons in these in vitro constructs project long, parallel, fasciculated axons to the periphery. In native tissue, axons of varying diameter and degree of myelination conduct sensory information back to the central nervous system at different velocities. Schwann cells support the sensory relay by myelinating axons and providing insulation for swifter conduction. Similarly, the three dimensional growth induced by this in vitro construct comprises axons of various diameters in dense, parallel orientation spanning distances up to 3 mm. Schwann cell presence and sheathing was observed in confocal and TEM imaging.

Although neuronal morphology is a useful indicator of phenotypic maturity, a more definitive sign of healthy neurons is their ability to conduct an action potential. Apoptosis alone is not a full measure of the neuronal health, as many pathological changes may occur before cell death manifests. Electrophysiological studies of action potential generation can determine whether the observed structures support predicted function, and the ability to measure clinically relevant endpoints produces more predictive results. Similarly, information gathered from imaging can determine quantitative metrics for the degree of myelination, while CAP measurement demonstrates the overall health of myelin and lends further insight into toxic and neuroprotective mechanisms of various agents or compounds of interest.

In some embodiments, the at least one agent comprises a small chemical compound. In some embodiments, the at least one agent comprises at least one environmental or industrial pollutant. In some embodiments, the at least one agent comprises one or a combination of small chemical compounds chosen from: chemotherapeutics, analgesics, cardiovascular modulators, cholesterol, neuroprotectants, neuromodulators, immunomodulators, anti-inflammatories, and anti-microbial drugs.

In some embodiments, the at least one agent comprises one or a combination of chemotherapeutics chosen from: Actinomycin, Alitretinoin, All-trans retinoic acid, Azacitidine, Azathioprine, Bexarotene, Bleomycin, Bortezomib, Capecitabine, Carboplatin, Chlorambucil, Cisplatin, Cyclophosphamide, Cytarabine, Dacarbazine (DTIC), Daunorubicin, Docetaxel, Doxifluridine, Doxorubicin, Epirubicin, Epothilone, Erlotinib, Etoposide, Fluorouracil, Gefitinib, Gemcitabine, Hydroxyurea, Idarubicin, Imatinib, Irinotecan, Mechlorethamine, Melphalan, Mercaptopurine, Methotrexate, Mitoxantrone, Nitrosoureas, Oxaliplatin, Paclitaxel, Pemetrexed, Romidepsin, Tafluposide, Temozolomide (Oral dacarbazine), Teniposide, Tioguanine (formerly Thioguanine), Topotecan, Tretinoin, Valrubicin, Vemurafenib, Vinblastine Vincristine, Vindesine, Vinorelbine, Vismodegib, and Vorinostat.

In some embodiments, the at least one agent comprises one or a combination of analgesics chosen from: Paracetoamol, Non-steroidal anti-inflammatory drugs (NSAIDs), COX-2 inhibitors, opioids, flupirtine, tricyclic antidepressants, carbamaxepine, gabapentin, and pregabalin.

In some embodiments, the at least one agent comprises one or a combination of cardiovascular modulators chosen from: nepicastat, cholesterol, niacin, scutellaria, prenylamine, dehydroepiandrosterone, monatepil, esketamine, niguldipine, asenapine, atomoxetine, flunarizine, milnacipran, mexiletine, amphetamine, sodium thiopental, flavonoid, bretylium, oxazepam, and honokiol.

In some embodiments, the at least one agent comprises one or a combination of neuroprotectants and/or neuromodulators chosen from: tryptamine, galanin receptor 2, phenylalanine, phenethylamine, N-methylphenethylamine, adenosine, kyptorphin, substance P, 3-methoxytyramine, catecholamine, dopamine, GABA, calcium, acetylcholine, epinephrine, norepinephrine, and serotonin.

In some embodiments, the at least one agent comprises one or a combination of immunomodulators chosen from: clenolizimab, enoticumab, ligelizumab, simtuzumab, vatelizumab, parsatuzumab, Imgatuzumab, tregalizaumb, pateclizumab, namulumab, perakizumab, faralimomab, patritumab, atinumab, ublituximab, futuximab, and duligotumab.

In some embodiments, the at least one agent comprises one or a combination of anti-inflammatories chosen from: ibuprofen, aspirin, ketoprofen, sulindac, naproxen, etodolac, fenoprofen, diclofenac, flurbiprofen, ketorolac, piroxicam, indomethacin, mefenamic acid, meloxicam, nabumetone, oxaprozin, ketoprofen, famotidine, meclofenamate, tolmetin, and salsalate.

In some embodiments, the at least one agent comprises one or a combination of anti-microbials chosen from: antibacterials, antifungals, antivirals, antiparasitics, heat, radiation, and ozone.

The present disclosure additionally discloses a method of measuring both intracellular and extracellular recordings of biomimetic neural tissue in a three-dimensional culture platform. Previously, electrophysiological experiments were undertaken in either dissociated surface-plated cultures or organotypic slice preparations, with limitations inherent to each method. Investigation in dissociated cell cultures is typically limited to single-cell recordings due to a lack of organized, multi-cellular neuritic architecture, as would be found in organotypic preparations. Organotypic preparations have intact neural circuitry and allow both intra- and extracellular studies. However, acute brain slices present a complex, simultaneous array of variables without the means to control individual factors and thus are inherently limited in throughput possibility.

Intracellular recording in in vitro three-dimensional cultures has been previously demonstrated. However, neuronal outgrowth was not spatially confined to an anatomically relevant structure supporting extracellular population investigation. A more biomimetic three dimensional neural culture is needed to allow examination of population-level electrophysiological behavior. The present disclosure supports whole-cell patch clamp techniques and synchronous population-level events in extracellular field recordings resulting from the confined neurite growth in a three dimensional geometry. Prior to the present disclosure, the measurement of these endpoints, directly analogous to clinical nerve conduction testing, had yet to be demonstrated for purely cellular in vitro studies.

Using the methods and devices disclosed herein, field recordings are used to measure the combined extracellular change in potential caused by signal conduction in all recruited fibers. The population response elicited by electrical stimulation is a CAP. Electrically evoked population spikes are graded in nature, comprising the combined effect of action potentials in slow and fast fibers. Spikes are single, cohesive events with swift onsets and short durations that are characteristic of CAPs or responses comprised purely of action potentials with quick signal conduction in the absence of synaptic input. The three-dimensional neural constructs disclosed by the present disclosure also support CAPs stimulated from farther distances along the neurite tract or channel, demonstrating the neural culture's ability to swiftly carry signals from distant stimuli much like an afferent peripheral nerve. The three dimensional neural cultures of the present disclosure support proximal and distal stimulation techniques useful for measuring conduction properties.

The present disclosure may be used with one or more growth factors that induce recruitment of numerous fiber types, as is typical in nerve fiber tracts. In particular, nerve growth factor (NGF) preferentially recruits small diameter fibers, often associated with pain signaling, as demonstrated in the data presented herein. It has been shown that brain derived neurotrophic factor (BDNF) and neurotrophic factor 3 (NT-3) preferentially support the outgrowth of larger-diameter, proprioceptive fibers. Growth-influencing factors like bioactive molecules and pharmacological agents may be incorporated with electrophysiological investigation to allow for a systematic manipulation of conditions for mechanistic studies.

The three-dimensional neural cultures created using the present disclosure may be used as a platform to study the mechanisms underlying myelin-compromising diseases and peripheral neuropathies by investigating the effects of known dysmyelination agents, neuropathy-inducing culture conditions, and toxic neuropathy-inducing compounds on the neural cultures. The present disclosure permits conduction velocity to be used as a functional measure of myelin and nerve fiber integrity under toxic and therapeutic conditions, facilitating studies on drug safety and efficacy. The incorporation of genetic mutations and drugs into neural cultures produced using the techniques disclosed herein may enable the reproduction of disease phenomena in a controlled manner, leading to a better understanding of neural degeneration and possible treatment therapies.

The present disclosure provides devices, methods, and systems involving production, maintenance, and physiological interrogation of neural cells in microengineered configurations designed to mimic native nerve tissue anatomy. In some embodiments, the devices and systems comprise one or plurality of cultured or isolated Schwann cells and/or one or a plurality of cultured or isolated oligodendrocytes in contact with one or a plurality of neuronal cells in a cell culture vessel comprising a solid substrate, said substrate comprising at least one exterior surface, at least one interior surface and at least one interior chamber; the shape of the interior chamber defined, at least in part, by the at least one interior surface and accessible from a point exterior to the solid substrate through at least one opening in the exterior surface; wherein soma of the one or plurality of neuronal cells are positioned at one end of the interior chamber and axons are capable of growing within the interior chamber along at least one length of the interior chamber, such that the position of a tip of an axon extends distally from the soma. In some embodiments, the interior surface of the solid substrate is in the shape of a cylinder or is substantially cylindrical, such that the soma from the neuronal cells are positioned proximal to the opening at one end of the cylindrical or substantially cylindrical interior surface and the axons of the neuronal cells comprise a length of cellular matter extending from a point at an ede of the soma to a point distal from the soma along the length of the interior surface. In some embodiments, the interior surface of the solid substrate is in the shape of a cylinder or is substantially cylindrical, such that the soma from the neuronal cells are positioned proximal to the opening at one end of the cylindrical or substantially cylindrical interior surface and the axons of the neuronal cells comprise a length of cellular matter extending from a point at an edge of the soma to a point distal from the soma along the length of the interior surface. In some embodiments, the interior surface of the solid substrate is in the shape of a cylinder or is substantially cylindrical, such that the soma from the neuronal cells are positioned proximal to the opening at one end of the cylindrical or substantially cylindrical interior surface and the axons of the neuronal cells comprise a length of cellular matter extending from a point at an edge of the soma to a point distal from the soma along the length of the interior surface; wherein, if the cell culture vessel comprises a plurality of neuronal cells, a plurality of axons extend from a plurality of somata (or soma) such that the plurality of axons define a bundle of axons capable of growth distally from the soma along the length of the interior surface. In some embodiments, the neuronal cells grow on and within the penetrable polymer. In some embodiments, one or a plurality of electrodes are positioned at or proximate to the tip of at least one axon and one or a plurality of electrodes are positioned at or proximate to the soma such that a voltage potential is established across the length of one or a plurality of neuronal cells.

It is another object of the disclosure to provide a medium to high-throughput assay of neurological function for the screening of pharmacological and/or toxicological properties of chemical and biological agents. In some embodiments, the agents are cells, such as any type of cell disclosed herein, or antibodies, such as antibodies that are used to treat clinical disease. in some embodiments, the agents are any drugs or agents that are used to treat human disease such that toxicities, effects or neuromodulation can be compared among a new agent which is a proposed mammalian treatment and existing treatments from human disease. In some embodiments, new agents for treatment of human disease are treatments for neurodegenerative disease and are compared to existing treatments for neurodegenerative disease. In the case of multiple sclerosis as a non-limiting example, the effects of a new agent (modified cell, antibody, or small chemical compound) may be compared and contrasted to the same effects of an existing treatment for multiple sclerosis such as Copaxone, Rebif, other interferon therapies, Tysabri, dimethyl fumarate, fingolimod, teriflunomide, mitoxantrone, prednisone, tizanidine, baclofen,

It is another object of the disclosure to employ unique assembly of technologies such as two-dimensional and three-dimensional microengineered neural bundles in conjunction with electrophysiological stimulation and recording of neural cell populations.

It is another object of the disclosure to provide a novel approach to evaluate neural physiology in vitro, using the compound action potential (CAP) as a clinically analogous metric to obtain results that are more sensitive and predictive of human physiology than those offered by current methods.

It is another object of this disclosure to provide microengineered neural tissue that mimics native anatomical and physiological features and that is susceptible to evaluation using high-throughput electrophysiological stimulation and recording methods.

It is another object of the present disclosure to provide methods of replicating, manipulating, modifying, and evaluating mechanisms underlying myelin-compromising diseases and peripheral neuropathies.

It is another object of the present disclosure to allow medium to high-throughput assay of neuromodulation in human neural cells for the screening of pharmacological and/or toxicological activities of chemical and biological agents.

It is another object of the present disclosure to employ unique assembly of technologies such as 2D and 3D microengineered neural bundles in conjunction with optical and electrochemical stimulation and recording of human neural cell populations.

It is another object of the present disclosure to quantify evoked post-synaptic potentials in a biomimetic, engineered thalamocortical circuits. Our observation of antidromically-generated population spike in neural tracts suggest that they are capable of population-level physiology, such as the conduction of compound action potentials and postsynaptic potentials.

It is another object of the present disclosure to utilize optogenetic methods, hardware and software control of illumination, and fluorescent imaging to allow for noninvasive stimulation and recording of multi-unit physiological responses to evoked potentials in neural circuits

It is anther object of the present disclosure to use the microengineered circuits in testing selective 5-HT reuptake inhibitors (SSRIs) and second-generation antipsychotic drugs to see if they alter their developmental maturation.

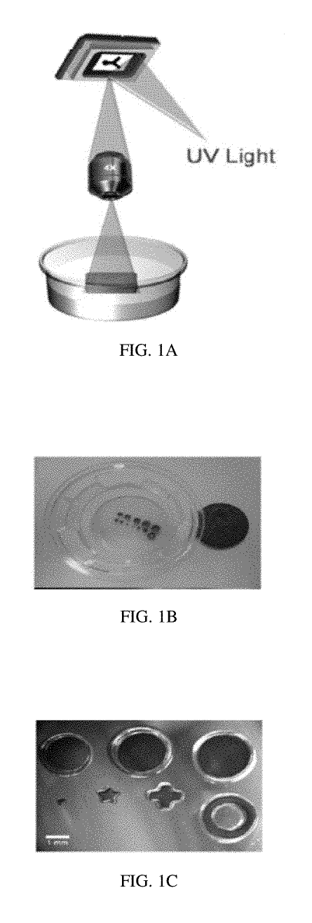

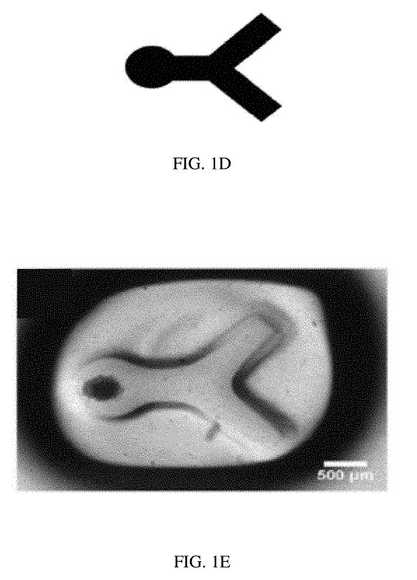

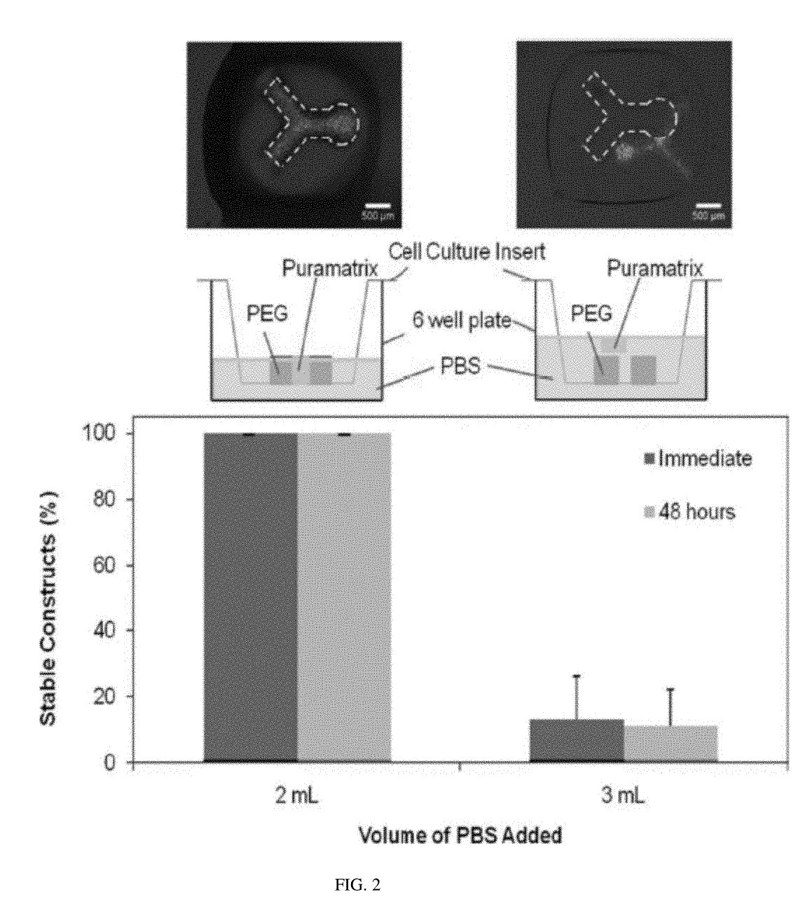

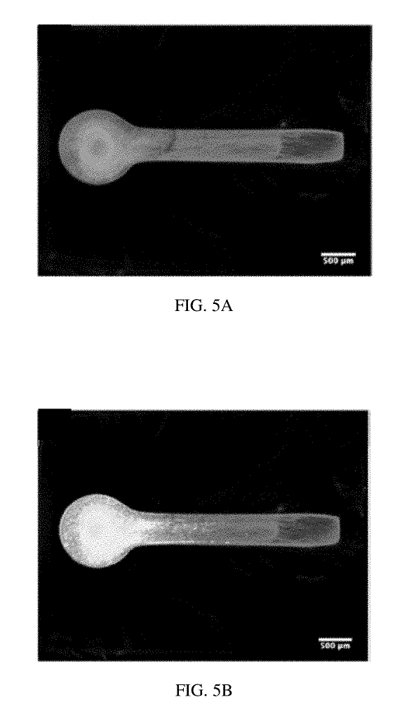

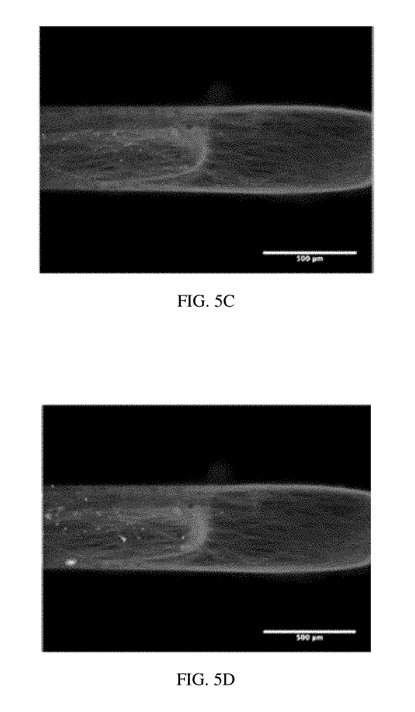

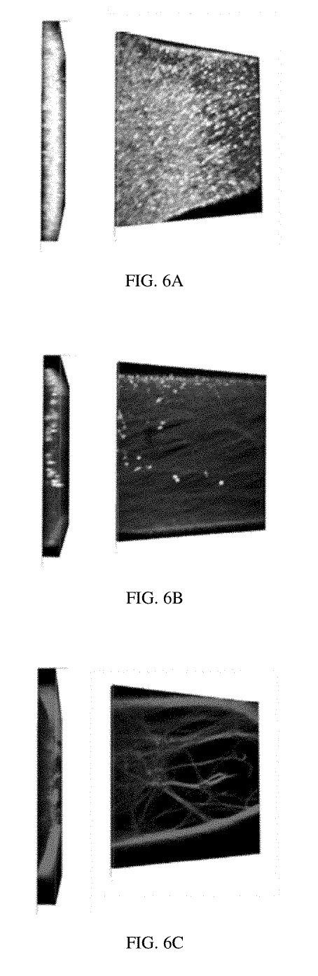

In one embodiment, projection photolithography using a digital micromirror device (DMD) is employed to micro pattern a combination of polyethylene glycol dimethacrylate and Puramatrix hydrogels, as shown in FIG. 1. This method enables rapid micropatterning of one or more hydrogels directly onto conventional cell culture materials. Because the photomask never makes contact with the gel materials, multiple hydrogels can rapidly be cured in succession, enabling fabrication of many dozens of gel constructs in an hour, without automation. This approach enables the use of polyethylene glycol (PEG), a mechanically robust, cell growth-restrictive gel, to constrain neurite growth within a biomimetic, growth conducive gel. In some embodiments, this growth-conducive gel may be Puramatrix, agarose, or methacrylated dextran. When embryonic dorsal root ganglion (DRG) explants are grown in this constrained three dimensional environment, axons grow out from the ganglion with high density and fasciculation, as shown in FIG. 5 and FIG. 6. The majority of axons appear as small diameter, unmyelinated fibers that grow to lengths approaching 1 em in 2 to 4 weeks. The structure of this culture model with a dense, highly-parallel, three dimensional neural fiber tract extending out from the ganglion is roughly analogous to peripheral nerve architecture. Its morphology may be assessed using neural morphometry, allowing for clinically-analogous assessment unavailable to traditional cellular assays.

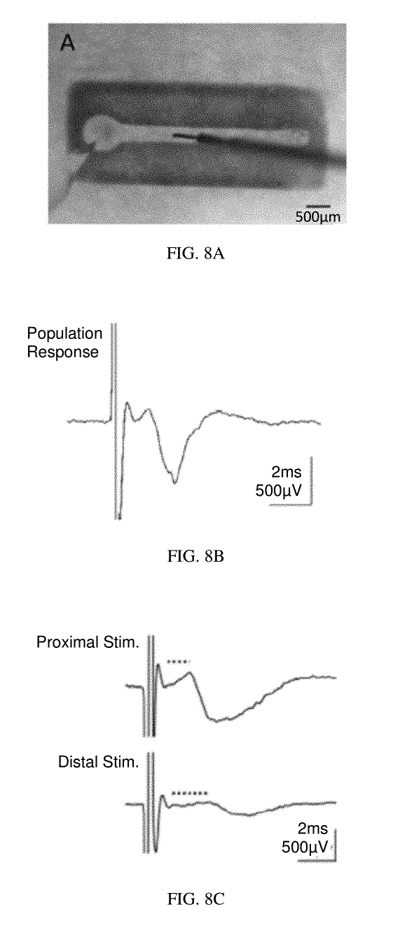

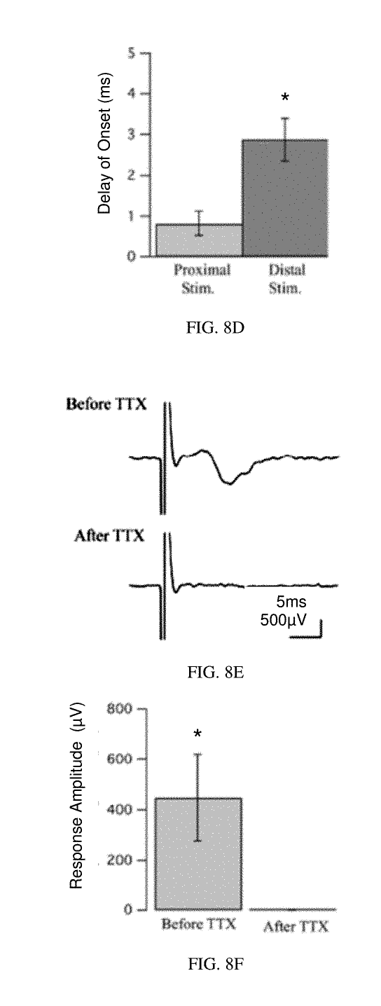

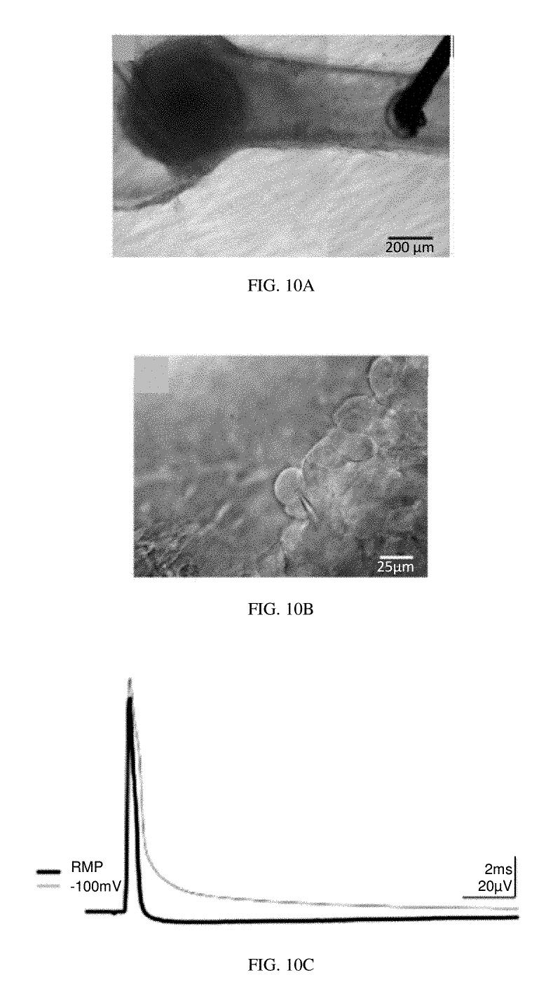

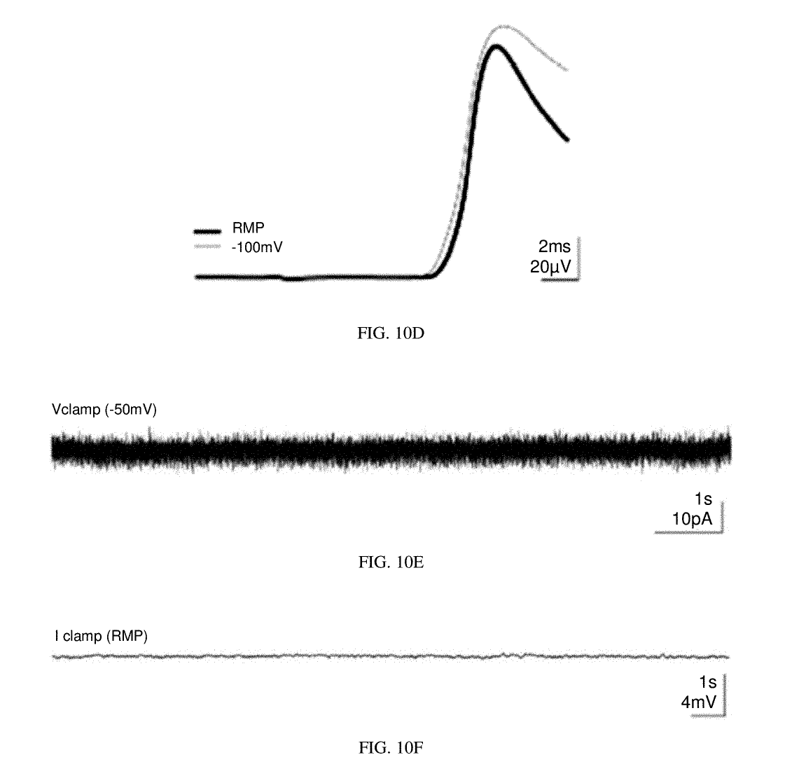

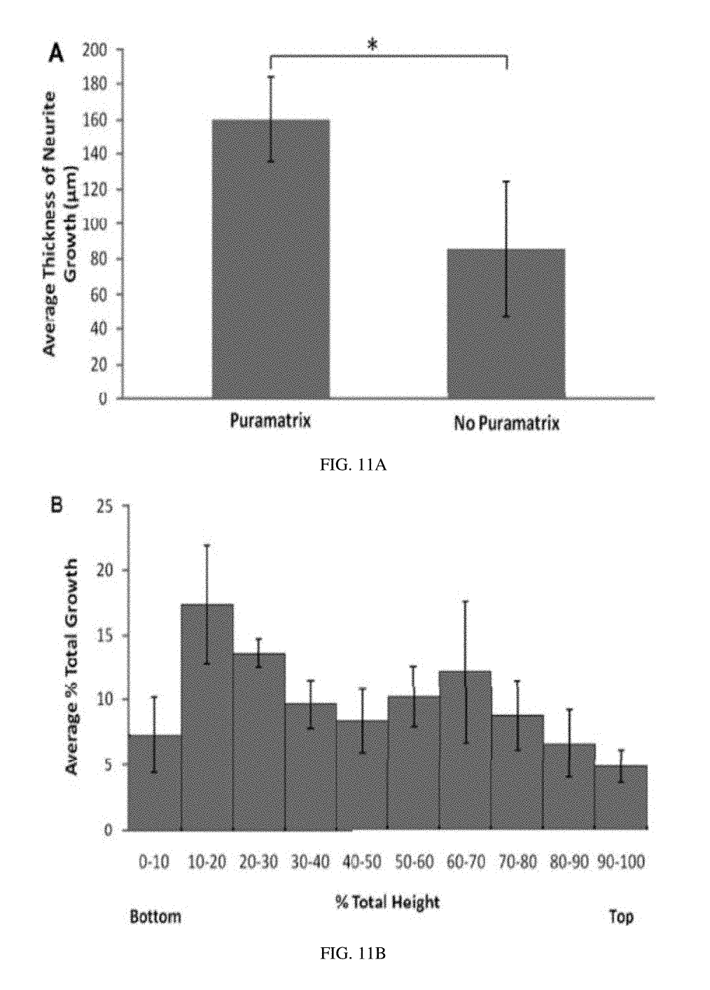

In a preferred embodiment, the culture model provides the ability to record electrically evoked population field potentials resulting from compound action potentials (CAPs). Example traces show the characteristic uniform, fast, short latency, population spike responses, which remain consistent with high frequency (100 Hz) stimulation, as seen in FIG. 8B. The CAPs are reversibly abolished by tetrodotoxin (TTX), as shown in FIGS. 8E and 8F, demonstrating that drugs can be applied and shown to have an effect. There is a measurable increase in delay to onset associated with distal tract stimulation, seen in FIGS. 8C and 8D. The responses are insensitive to neurotransmitter blockers, indicating the evoked responses are primarily CAPs rather than synaptic potentials, shown in FIG. 10. Embryonic DRG cultures have been used effectively as models of peripheral nerve biology for decades. While extremely useful as model systems, conventional DRG cultures are known to be poorly predictive of clinical toxicity when assessed with traditional cell viability assays. While it is possible to perform single-cell patch clamp recording in DRG cultures, there are no reports of recording CAPs, due to the lack of tissue architecture. In a preferred embodiment, the present disclosure provides the ability to assess tissue morphometry and population electrophysiology, analogous to clinical histopathology and nerve conduction testing.

In some embodiments, the present disclosure uses human neural cells to grow nerve tissue in a three dimensional environment in which neuronal cell bodies are bundled together and located in distinct locations from axonal fiber tracts, mimicking native nerve architecture and allowing the measurement of morphometric and electrophysiological data, including CAPs. In some embodiments, the present disclosure uses neuronal cells and glial cells derived from primary human tissue. In other embodiments, neuronal cells and glial cells may be derived from human stem cells, including induced pluripotent stem cells.

In another embodiment. the present disclosure uses conduction velocity as a functional measure of neural tissue condition under toxic and therapeutic conditions. Information on degree of myelination, myelin health, axonal transport, mRNA transcription and neuronal damage may be determined from electrophysiological analysis. Taken in combination with morphometric analysis of nerve density, myelination percentage and nerve fiber type, mechanisms of action can be determined for compounds of interest. In some embodiments, the devices, methods, and systems disclosed herein may incorporate genetic mutations and drugs to reproduce disease phenomena in a controlled manner, leading to a better understanding of neural degeneration and possible treatment therapies.

The following examples are meant to be non-limiting examples of how to make and use the embodiments disclosed in this application. Any publications disclosed in the examples or the body of the specification are incorporated by reference in their entireties.

### EXAMPLES

### Example 1: Growth and Physiological Assessment of Neural Tissues in a Hydrogel Construct (Non-Prophetic)

### A. Materials and Methods

Dynamic Mask Projection Photolithography.

Hydrogel micropatterns were formed via projection photolithography. A DMD development kit (Discovery™ 3000, Texas Instruments, Dallas, Tex.) with USB computer interface (ALP3Basic) served as a dynamic mask by converting digital black and white images to micromirror patterns on the DMD array, in which individual mirrors may be turned “on” or “off” by rotating the angle of reflection from +12° to −12°, respectively. Ultraviolet (UV) light filtered at 320-500 nm from an OmniCure 1000 (EXFO, Quebec, Canada) Hg vapor light source was collimated with an adjustable collimating adapter (EXPO) and projected onto the DMD array. The reflected light was projected through a 4× Plan Fluor objective lens (Nikon Instruments, Melville, N.Y.) with numerical aperture 0.13 and focused directly onto a photocrosslinkable hydrogel solution, as shown by FIG. 1A. The iris of the UV light source was adjusted to maintain an irradiance output of 5.0 watts/cm2 as measured with a radiometer (EXPO). Hydrogel solutions were cured for about 55 seconds, inducing crosslinking through free radical chain reaction. Unlike previous reports, this method initiated crosslinking throughout the bulk with a single irradiation, negating the need for a layer-by-layer approach.

Formation of Dual Hydrogel Constructs.

Hydrogel polymerization was performed as previously described for dynamic mask projection photolithography. The photocrosslinkable solution was made by diluting polyethylene glycol dimethacrylate (PEG) with average molecular weight (MW) 1000 Da (Polysciences, Warrington, Pa.) to 10% (w/v) in either PBS or growth medium with 0.5% (w/v) Irgacure 2959 (1-2959) (Ciba Specialty Chemicals, Basel, Switzerland) as a photoinitiator. The concentration and molecular weight of PEG was chosen based on previously published data to minimize cell adhesion and to maximize hydrogel adherence to the polymerization surface. Micropatterned PEG constructs were crosslinked directly onto one of three types of permeable cell culture inserts: polyester, polycarbonate, and collagen-coated PTFE Transwell® Permeable Supports (Corning, Corning, NY) with 24 mm diameter membranes and 0.411 m pores. Inner walls of the culture inserts, not the membranes themselves, were treated with Rain-X® (SO PUS Products, Houston, Tex.) to reduce meniscus effect of PEG solution. Each support was placed on the stage of an inverted microscope positioned directly below the lithography projection lens. After crosslinking, supports were rinsed, removing excess uncrosslinked PEG solution, and the micropatterned PEG remained attached to the surface. Hydration of PEG gel was maintained in buffered saline solution (4° C.) if not used immediately.

A self-assembling peptide gel, Puramatrix (BD Biosciences, Bedford, Mass.), was diluted to 0.15% (w/v) in deionized H₂O prior to use and was supplemented pregelation with 1 μg/mL soluble laminin (Invitrogen, Carlsbad, Calif.) when used for neurite outgrowth experiments. This second gel has also been substituted with agarose and methacrylated versions of hyaluronic acid, heparin, and dextran. Both the concentration of Puramatrix and the addition of laminin were according to manufacturer's instructions for neural application. Using a pipette, this solution was carefully added to voids within the micropatterned PEG hydrogels. Contact with salt solution hydrating the PEG gel induced self-assembly of the Puramatrix, which remained confined within the PEG geometry. Puramatrix gelation was maintained by incubating at 37° C. and 5% C02.

Tissue Harvesting and Culture.

NIH guidelines for the care and use of laboratory animals (NIH Publication #85-23 Rev. 1985) were observed. Embryonic day 15 (E-15) pups were removed from timed-pregnant Long Evans rats (Charles River, Wilmington, Mass.) and placed in Hank's Balanced Salt Solution. Spinal columns were isolated from embryos, from which dorsal root ganglia (DRG) were harvested and placed in Neurobasal Medium supplemented with nerve growth factor (NGF), 10% fetal bovine serum (FBS), and penicillin/streptromycin (P/S) (Invitrogen) to promote adhesion. After adhesion, DRGs were placed on collagen-coated cell culture inserts and maintained in an incubator at 37° C. and 5% C02 with B-27 and L-glutamine replacing FBS for growth medium.

Primary DRG neurons were obtained through dissociation of DRGs by tripsinization and trituration, followed by the subsequent removal of supportive cells using fluorodeoxyuridine and uridine (3Day treatment). Cells were then suspended in Puramatrix according to the manufacturer's protocol at a concentration of 3×10⁵ cells/mL. The cell suspension was added, at a volume sufficient to obtain about 480 μm thick hydro gels, to either 24 well cell culture inserts or 24 well tissue culture plates (Corning), and self-assembly was initiated upon addition of growth medium (n=4). The constructs were incubated for about 48 hours before testing cell viability.

Neurite Outgrowth in Dual Hydrogels.

Collagen-coated PTFE cell culture inserts were soaked overnight in adhesion medium to hydrate the membrane. Four DRGs were then placed on the surface of an insert and allowed to adhere for about 2 hours before the medium was replaced with 500 μL of 10% PEG in growth medium as described earlier, without FBS. This volume may be adjusted to vary the thickness of the PEG constructs. The DMD was illuminated with a visible light source to aid alignment of the projected mask with each adhered DRG. The visible light source was then replaced by the UV source and the PEG hydrogel crosslinked around the tissue explant. DRG-containing PEG constructs were washed three times with PBS to remove any uncrosslinked PEG solution. When applicable, modified Puramatrix was added to the void inside the PEG, and to induce Puramatrix self-assembly, 1.5 mL of growth medium was introduced beneath the insert. Constructs referred to as without Puramatrix were made as described above except without the addition of Puramatrix, thereby restricting DRGs to the two-dimensional environment of the collagen-coated PTFE membrane. Constructs were maintained in an incubator at 37° C. and 5% C0₂ for 7 days, and medium was changed after the second and fifth days.

Constructs were prepared and visualized for morphology, viability, neurite outgrowth, and containment. If no neurites were visualized growing on or outside the PEG void, the sample was considered to have contained growth. This was done in identical PEG constructs both with and without Puramatrix added, and twelve trials for each condition were attempted for the five different PEG heights described above. Trials were thrown out in cases of incomplete PEG polymerization or lack of DRG adhesion.

Specimen Preparation and Visualization.

Live specimens were evaluated for viability with a Live/Dead® assay (Invitrogen) per manufacturer's instructions. For cell suspensions in Puramatrix, wide field fluorescent images were captured at multiple focal planes throughout the depth of the gel in three different areas of each hydrogel specimen. Standard deviation projections were then analyzed for cell viability in both cell culture inserts and tissue culture plates by counting calcein AM (live marker) and ethidium homodimer-1 (dead marker), giving a total of 12 samples per condition. Specimens evaluated with immunohistochemistry were fixed in 4% paraformaldehyde for about 2 hours. Cell nuclei were stained with DAPI Nucleic Acid Stain (Molecular Probes) per manufacturer's instructions. Neurites were stained using mouse monoclonal [2G10] to neuron specific β III tubulin primary antibody and goat-antimouse IgG-H & L (CY2) secondary antibody, and dendrite staining was carried out using rabbit polyclonal to MAP2 primary antibody and donkey-antirabbit IgG Dylight 594 secondary antibody (AbCam, Cambridge, Mass.). Each step was carried out in PBS with 0.1% Saponin and 2.0% BSA overnight followed by three washes in PBS with 0.1% Saponin. Bright field and conventional fluorescent images were acquired with a Nikon AZ1 00 stereo zoom microscope (Nikon, Melville, N.Y.) equipped with fluorescence cubes, while confocal images were acquired using a Zeiss LSM 510 Meta microscope (Zeiss, Oberkocken, Germany). Average depths of β-III labeled structures were calculated from confocal images to measure the distance between the first and last focal plane containing fluorescence (n=7). Image processing was performed with Image J (National Institutes of Health, Bethesda, Md.), and V3D software (Howard Hughes Medical Institute, Ashburn, Va.) used to visualize confocal image stacks in 3D.

The proportion of neurite growth over the depth of the gel was quantified from pixel counts of manually thresholded confocal slices. Confocal slices were binned in 10% increments of total depth, and the measured fluorescence of binned slices was compared with the total measured for each z-stack to give the proportion of neurite growth throughout the depth of the construct (n=3). The VolumeJ plugin was used to create depth coded z-stack projection of neurite growth. Confocal z-stacks were acquired through the maximum depth of visible neurite growth (186 μm) with 3.0 μm thick slices (1024×1024×63) for both Puramatrix and non-Puramatrix containing constructs. The Z Code Stack function with spectrum depth coding LUT was used to add color, and the stacks were merged using a standard deviation z projection. Last, z-stacks were despeckled to remove background noise. Cryogenic scanning electron microscopy (Cryo-SEM) was performed by freezing specimens in slushed liquid nitrogen and imaging with a Hitachi 54800 Field Emission SEM (Hitachi, Krefeld, Germany) and Gatan Alto 2500 Cryo System (Gatan, Warrendale, Pa.) at 3 kV and −130° C.

Incorporation of Dorsal Root Ganglia Explants.

All animal handling and tissue harvesting procedures were performed under observation of guidelines set by NIH (NIH Publication #85-23 Rev. 1985) and the Institutional Animal Care and Use Committee (IACUC) of Tulane University. Neural explants were incorporated into dual hydrogel constructs as described above. Briefly, 6 well collagen-coated PTFE cell culture inserts were soaked overnight in adhesion media consisting of Neurobasal medium supplemented with penicillin/streptomycin, nerve growth factor (NGF), 10% fetal bovine serum (FBS), and L-glutamine (Gibco-Invitrogen, Carlsbad, Calif.). Four dorsal root ganglia (DRG) isolated from Long-Evans rat embryonic day 15 pups (Charles River, Wilmington, Mass.) were placed on a hydrated cell culture insert and incubated in adhesion media for about 2 hours at 37° C. and 5% CO₂ to adhere. Adhesion media were then replaced by 500 μl of 10% PEG/0.5% Irgacure 2959 in PBS for construct polymerization.

The projected photomask pattern for the PEG construct was aligned around an adhered DRG using visible light and an inverted microscope. UV light was used to project the same photomask for 55 seconds, as described above, and effectively confined the DRG within a polymerized PEG construct. The time tissue cultures spent outside of the biosafety cabinet was kept to a minimum to help prevent contamination, and uncrosslinked hydrogel solution was rinsed 3 times with PBS containing 1% penicillin/streptomycin (Gibco-Invitrogen, Carlsbad, Calif.) to remove unpolymerized PEG solution and improve culture sterility. Excess PBS was removed from patterned voids inside PEG and Puramatrix was carefully pipetted into the remaining space. The insert containing the dual hydrogel constructs, each with a live DRG explant, was immediately placed in 1.5 ml of growth media (Neurobasal medium supplemented with NGF, penicillin/streptomycin, L-glutamine, and B27; Gibco-Invitrogen, Carlsbad, Calif.) to initiate the self-assembly of the Puramatrix and maintained at 37° C. and 5% CO₂, with media changes about every 48 hours. Experiments were initiated after 7 days to permit neurite outgrowth and neuronal maturation.

Immunocytochemistry.

Specimens evaluated with immunohistochemistry were fixed in 4% paraformaldehyde (Electron Microscopy Sciences, Hatfield, Pa.) for about 2 hours at 37° C. Cell nuclei were stained with DAPI nucleic acid stain according to manufacturer's instructions (Molecular Probes, Eugene, Oreg.). Neurites were tagged using mouse monoclonal [2G 10] neuron-specific β-III tubulin primary antibody (1:200), followed by fluorescent tagging with Cy3.5-conjugated goat anti-mouse immunoglobulinG (H+L) secondary antibody (1:100; Abeam, Cambridge, Mass.). Glial cells were stained using rabbit polyclonal S 100-specific primary antibody (1:500, Abeam, Cambridge, Mass.) and Cy2-conjugated goat anti-rabbit immunoglobulinG (H+L) secondary antibody (1:100, Jackson ImmunoResearch Laboratories, Westgrove, Pa.). Antibody tagging steps were carried out in PBS with 0.1% saponin and 2% bovine serum albumin (Sigma-Aldrich, St. Louis, Mo.) overnight at 4° C., followed by three 10-minute washes in PBS with 0.1% saponin at room temperature.

For constructs stained for myelin, neurites were tagged using mouse monoclonal [2G 10] neuron-specific β-III tubulin primary antibody (1:200), followed by fluorescent tagging with Cy2-conjugated goat anti-mouse immunoglobulinG (H&L) secondary antibody (1:500; Abeam, Cambridge, Mass.). Myelin was stained using Fluoromyelin™ Red Fluorescent Myelin Stain (Molecular Probes, Eugene, Oreg.) for 40 minutes according to manufacturer's recommended preparation.

Fluorescence Microscopy and Image Processing.

Bright field and conventional fluorescent images were acquired with a Nikon AZ100 stereo zoom microscope using I× and 2× objectives (Nikon, Melville, N.Y.), while confocal images were taken using a Leica TCS SP2 laser scanning microscope and 20× objective (Leica Microsystems, Buffalo Grove, Ill.). Confocal z-stacks were acquired through the maximum depth of visible neurite growth with thicknesses ranging between 55-65 μm imaged over 20 slices, each 512×512. Image processing was performed using ImageJ (National Institutes of Health, Bethesda, Mass.). For color coding depth in confocal z-stacks, the Z Code Stack function with a Rainbow LUI was applied using the MacBiophotonics Plugin package for ImageJ. Projections of z-stacks were taken as maximum intensity projections. V3D-Viewer software (Janelia Farm Research Campus, Howard Hughes Medical Institute, Ashburn, Va.) allowed 3D rendering and visualization of the confocal z-stack images.

Transmission Electron Microscopy.

Transmission electron microscopy was used to qualitatively assess morphology, spatial distribution, and nanoscale features of neural cultures. After 7 days in vitro, constructs were fixed in 4% paraformaldehyde for about 2 hours at 37° C., washed three times for 10 minutes with PBS, and sectioned to reveal regions of interest. Post-fixation using 1% OsO₄ for about 1 hour and 2% uranyl acetate for about 30 minutes was performed in limited-light settings with three 10 minute PBS washes in between. The samples were dehydrated with ethanol (50, 70, 95, and 2×100%, about 30 minutes each) and embedded in 1:1 propylene oxide-spurr resin for about 45 minutes and 100% spur resin overnight (Low Viscosity Embedding Kit, Electron Microscopy Sciences, Hatfield, Pa.). Polymerization of specimens occurred at 70° C. over 24 hours.

Embedded samples were trimmed and sliced with thicknesses varying from 80 nm to 100 nm using a Reichert Ultracut S ultratome (Leica Microsystems, Buffalo Grove, Ill.) and Ultra 45° diamond knife (Diatome, Fort Washington, Pa.). Slices were placed on Formvar carbon-coated copper grids with 200 mesh and stained with 2% uranyl acetate and 0.1% lead citrate (about 20 minutes each). Samples were mounted on a single-tilted stage and examined with a FEI Tecnai G2 F30 Twin transmission electron microscope (FEI, Hillsboro, Oreg.) using an accelerator voltage of 200 kV. Images were taken at 3,000×-20,000× magnifications with 4000×4000 pixel resolution. All materials and reagents used for sample preparation were obtained from Electron Microscopy Sciences (Hatfield, Pa.).

Field Potential Recording.

After 7 days in vitro, dual hydrogel constructs containing live DRG explants were transferred to an interface chamber held at room temperature and perfused with bicarbonate buffered artificial cerebrospinal fluid (ACSF) made of, in mM, 124 NaCl, 5 KCl, 26 NaHC03, 1.23 NaHzP04, 4 MgS04, 2 CaClz, and 10 glucose. ACSF was bubbled with 95% Oz, 5% COz at all times to maintain consistent oxygenation and pH. Constructs were stained for contrast with 1% Bromophenol Blue (Sigma-Aldrich, St. Louis, Mo.) and visualized using an SMZ 745 stereomicroscope (Nikon, Melville, N.Y.). Thin-walled borosilicate glass pipettes (OD=1.5, ID=I.6; Warner Instruments, Hamden, Conn.) were pulled to resistances between about 3 and about 7 MQ using a P-97 Flaming/Brown micropipette puller (Sutter Instrument Co., Novato, Calif.) and backfilled with ACSF.

As shown in FIG. 8A, recording electrodes were placed near cell somata in the vicinity of each ganglion, and constructs were stimulated with a concentric bi-polar electrode (CBARB75, FHC, Bowdoin, Me.) at varying distances away from the ganglion along neurite tracts. An Axopatch-1 C amplifier (Molecular Devices, Sunnyvale, Calif.) coupled with an isolated pulse stimulator (Model 2100; A-M Systems, Sequim, Wash.), PowerLab 26T digitizer (AD Instruments, Colorado Springs, Colo.), and LabChart software (AD Instruments, Colorado Springs, Colo.) was used for recording, stimulating, and data acquisition. Recordings were filtered at 5 kHz, displayed on Tektronix oscilloscopes, and analyzed offline using custom written routines in Igor Pro (WaveMetrics, Portland, Oreg.). Standard deviations were calculated when appropriate. The statistical values were calculated using 2-tailed, paired/-tests with a p value<0.05 considered significant. All values are reported with errors as standard error of the mean (SEM).

20 μM DNQX (6,7-dinitroquinoxaline-2,3Dione) and 50 μM APV (2R)-amino-5-phosphonopentanoate) were used to identify and block synaptic activity. 0.5 μM tetrodotoxin (TTX) was used to as a complete blockade of Na+ channel activity. All drugs and salts used in experimental solutions were obtained from Tocris (Minneapolis, Minn.) and Sigma-Aldrich (St. Louis, Mo.) respectively.

Whole-Cell Patch Clamp Recording.

After 7 days in vitro, constructs were transferred to a submersion recording chamber at room temperature and allowed to equilibrate for 20 minutes. Bicarbonate-buffered ACSF solution (containing, in mM, 124 NaCl, 5 KCl, 26 NaHCO₃, 1.23 NaH₂PO₄, 1.5 MgCh, 2 CaCh, and 10 glucose) was bubbled with 95% 0 2, 5% C02 at all times to maintain consistent oxygenation and pH. For voltage clamp recordings, borosilicate glass pipettes were filled with a cesium-substituted intracellular solution containing, in mM, 120 CsMeS03, 1 NaCl, 0.1 CaCh, 2 ATP, 0.3 GTP, 10 HEPES, and 10 EGTA. For current damp recordings, pipettes were filled with a potassium gluconate based internal solution containing, in mM, 120 Kgluconate, 10 KCl, 10 Hepes, 10 D-sorbitol, 1MgCh*6H20, 1NaCl, 1 CaCh, 10 EGTA, 2 ATP. Pipette resistances ranged from about 4 to about 7 MQ. Series access resistance ranged from about 7 to about 15 MQ and was monitored for consistency. For evoked action potential recordings, concentric bipolar stimulating electrodes (CBARC75, FHC, Bowdoin, Me.) were placed in the afferent fibers of the DRG, and after attaining a whole-cell patch, action potentials were evoked using minimum stimulation necessary, typically <0.01 mA. Placement of recording and stimulating electrodes is shown in FIG. 10A below.

DRGs were visualized with a BX61 WI Olympus upright microscope (Olympus, Center Valley, Pa.) with live differential interference contrast (DIC) imaging. Whole-cell recordings were made with a PC-505B patch clamp amplifier (Warner Instruments, Harnden, Conn.). Signals were digitized with a PowerLab 26T digitizer and collected with Lab Chart acquisition software (AD Instruments, Colorado Springs, Colo.). Signals were amplified, sampled at 20 kHz, filtered to 2 kHz, and analyzed using custom written routines in Igor Pro (WaveMetrics, Portland, Oreg.).

Rat Ganglion Explant and Electrophysiology Assessment.

Rat E-15 dorsal root ganglion explants were cultured in dual hydrogel constructs micropatterned with a dynamic mask projection lithography method described above. Neural constructs were incubated for 1 week or 2 weeks, and neurite outgrowth was confined to narrow tracts filled with Puramatrix, measuring about 200 μm in diameter, about 400 μm thick, and up to about 2 mm in length. Constructs were placed on an interface chamber perfused with bicarbonate-buffered ACSF solution, and electrophysiology was assessed with extracellular field potential electrodes. Recording electrodes were placed near cell somata in the vicinity of each ganglion, and constructs were stimulated with a bi-polar electrode at varying distances away from the ganglion along neurite tracts.

### B. Results

Neurite Outgrowth in Dual Hydrogels.

The PEG thickness necessary to constrain neurite growth was investigated by culturing DRG explants in constructs with increasing thicknesses. Containment was measured here because it was crucial to the ability of this system to function reliably as an in vitro model. Impartial polymerization frequently occurred with 233 μm thick PEG, leading to unusable constructs. Additionally, throughout the polymerization process, some DRG detached from the surface of the membrane, leading to a lower than expected number of trials for analysis. For the constructs containing Puramatrix, a distinct increase in the containment of neurites was seen as gel thickness increased, as shown in Table 1. At a thickness of 233 μm, no constructs limited the growth of neurites. The rates of containment for the subsequent heights of 368, 433, 481, and 543 μm were: 10%, 22.2%, 63.6%, and 87.5%. Overall, higher percentages of containment were seen in constructs lacking Puramatrix. Neurites appeared able to grow over the sloping PEG walls at certain thicknesses in both groups, but in constructs without Puramatrix, there was more efficient containment at similar heights, other than 534 μm, and more effective containment by a lower height than in PEG with Puramatrix, as listed in Table 1.

TABLE 1
Neurite Growth Containment as a Function of Hydrogel Thickness

| | Volume (μL) | Thickness (μm) | n | % Contained |
|---|---|---|---|---|
| Puramatrix | 350 | 233 ± 24 | 6 | 0.0% |
| | 400 | 368 ± 32 | 10 | 10.0% |
| | 450 | 433 ± 19 | 9 | 22.2% |
| | 500 | 481 ± 14 | 11 | 63.6% |
| | 550 | 543 ± 9 | 8 | 87.5% |
| Non-Puramatrix | 350 | 233 ± 24 | 5 | 40.0% |
| | 400 | 368 ± 32 | 10 | 30.0% |
| | 450 | 433 ± 19 | 6 | 83.3% |
| | 500 | 481 ± 14 | 10 | 90.0% |
| | 550 | 543 ± 9 | 8 | 87.5% |

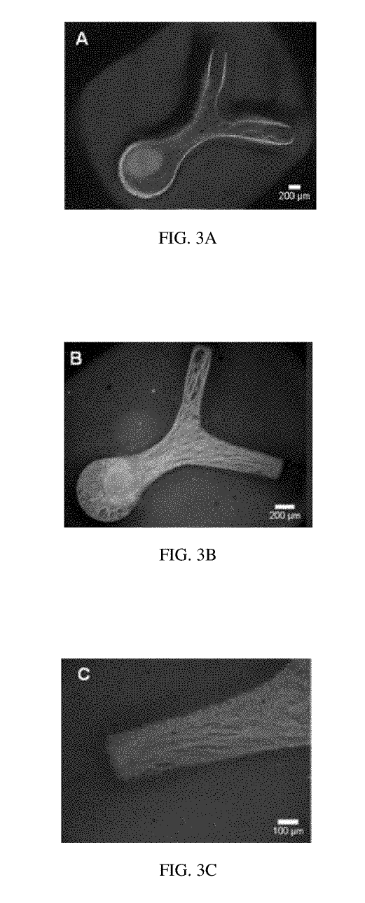

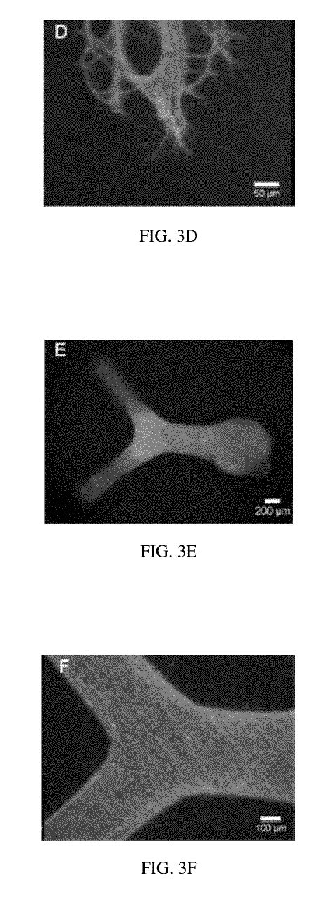

In an effort to balance void size resolution and pattern fidelity with neurite containment, subsequent neurite growth experiments were carried out in constructs with an average PEG thickness of 481 μm (500 μL solution). Constructs monitored for cell viability after 5 days showed an overwhelming amount of live cells, with an extremely small portion of dead cells located in the DRG itself, as shown by FIG. 3A. After fixation and staining at 7 days, neurites and migrating cells were constrained by the geometry of the PEG hydrogel, as suggested by dual labeling with β-III tubulin and DAPI, seen in FIG. (B. Neurite outgrowth was consistently robust and all labeled structures were concentrated inside the Puramatrix portion of the dual hydrogel, shown by FIGS. 1A-1E and FIGS. 5A-5D. Additionally, MAP2 antibody labeling suggests that a substantial portion of the neurite growth in the constructs appeared to be dendritic, as seen in FIG. 3E. Growth appeared to occur first along the boundary between the two gels, as is evident in FIG. 3A. However, behind the neurites extending along the channel, growth was seen filling in the inner space between the PEG, also shown in FIG. 3A. Images of leading neurite growth in the three-dimensional bulk of the Puramatrix showed a tendency to grow in random directions, as shown by FIG. 3D.

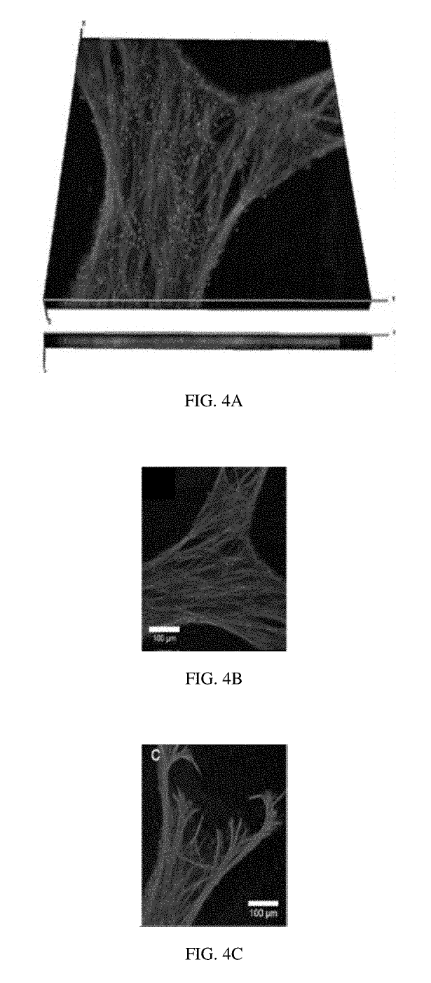

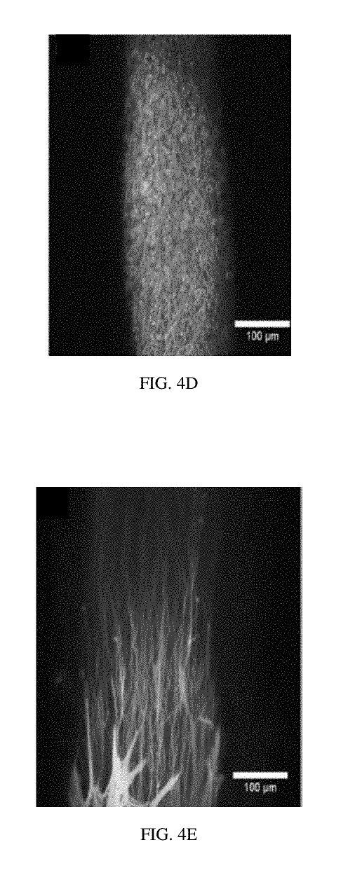

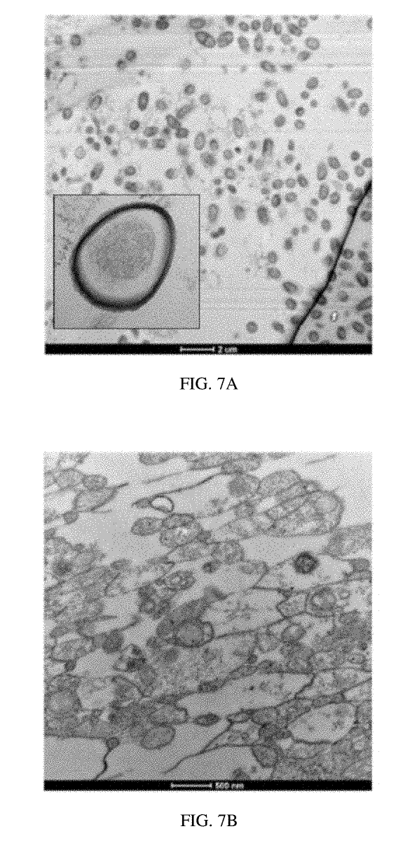

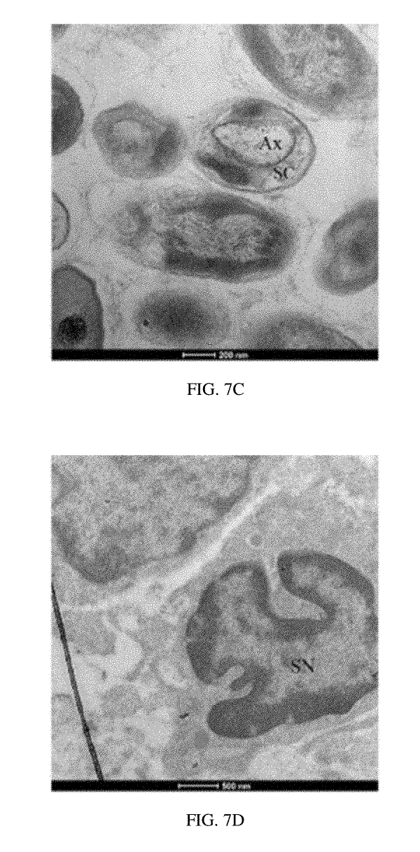

In direct contrast, neurites growing along the surface of the cell culture insert oriented themselves obliquely, apparently closely following the fibers of the insert membrane, as shown in FIG. 3F. Outgrowth was observed to fan out at the bifurcation point with no apparent preference in direction. A considerable amount of branching and fasciculation was observed, which was especially apparent at the leading edge of growth, as seen in FIG. 3D. In about 7 days, outgrowth was seen throughout the length of the channels. Significantly more growth was observed in Puramatrix-filled constructs, as compared with the apparently abortive limited growth seen in constructs without Puramatrix, as shown by FIGS. 4B and 4C. Confocal imaging confirmed that neurite growth occurred in three dimensions, as shown in FIGS. 7A-7D. The average thickness of β-III labeled structures was 159.8±23.9-μm thick in Puramatrix containing constructs, while the average thickness in constructs without Puramatrix was 85.4±38.6 μm, a difference which was found to be statistically significant and is shown by FIG. 11A, p<0.005.

FIG. 4D represents an example of growth in a construct lacking Puramatrix, where growth appeared crowded and neurites grew to a maximum height of 54.0 μm. Neurite growth in constructs without Puramatrix was visualized growing along the membrane of the collagen coated PTFE, with no growth occurring in the PEG itself. Alternatively, FIG. 4E demonstrates neurite growth in a dual hydrogel construct, with notably less neurite crowding observed, and individual neurites growing through Puramatrix in multiple focal planes, reaching a maximum height of 120.0 μm. FIG. 11B further demonstrates that neurite growth was not confined to either the membrane or the top surface of the Puramatrix, as only 7.3±2.9% and 4.9±1.3% of total growth was seen in the bottom and top 10% of the slices, respectively. Unlike the neurites, DAPI staining indicated migrating cells were not influenced to migrate into Puramatrix, remaining confined near the support surface, as shown by FIG. 4A, although previous research suggests that glial cell migration and neurite growth often occur together.

Spatial and Morphological Characteristics of Three-Dimensional Neural Cultures.

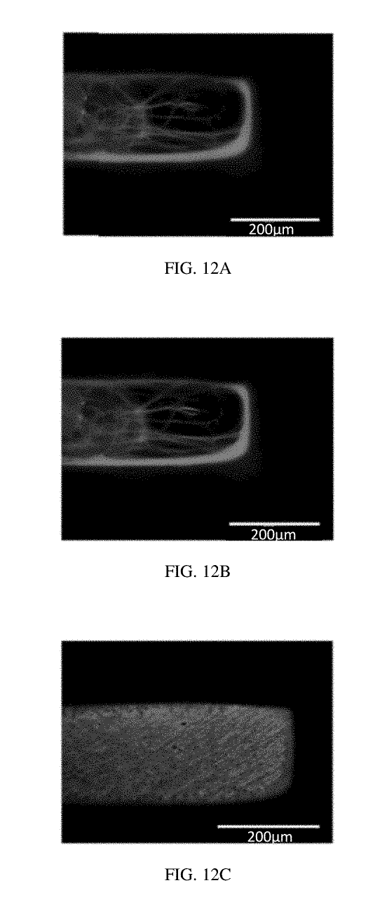

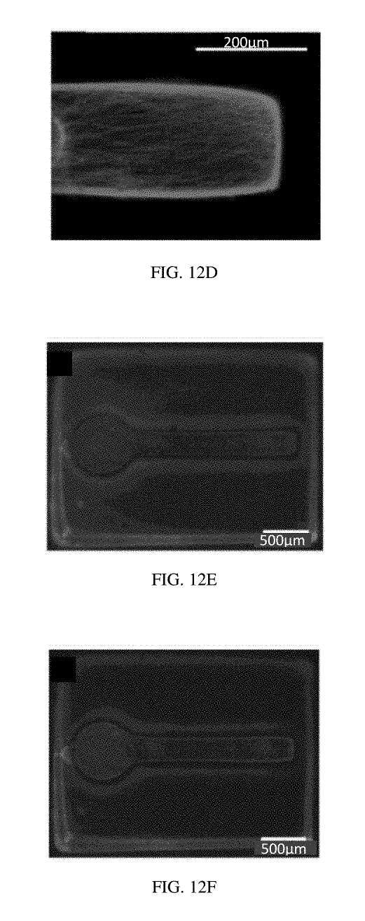

The present disclosure discloses an in vitro three-dimensional neural culture that approximates the cyto- and macro-scale architecture of native afferent peripheral nervous tissue. The three-dimensional neural constructs consist of DRG tissue explants cultured on the surface of a cell culture insert that are contained by PEG constructs that permit growth within patterned voids filled with Puramatrix. Narrow tracts guiding neurite growth from the ganglion along the x-axis measure about 490 μm in diameter, up to about 400 μm thick, and about 3 mm in length. A three-dimensional dual hydrogel construct containing DRG neurons, glia, and neurite growth is shown after 7 days in vitro in FIGS. 12A-12D.

The neurites and supportive glial cells were effectively constrained by the geometry of the PEG hydrogel. Simultaneous labeling with anti-β-III tubulin, anti-S100, and DAPI confirmed outgrowth after 7 days in vitro was consistently robust and all labeled structures were within the Puramatrix portion of the construct, as shown in FIGS. 5A and 5B. Presence and migration of supportive cells, including glial cells, spans up to three-quarters of the length of the channel, nearly 1.875 mm away from the ganglion as measured from the start of the straight channel, as shown in FIGS. 5C and 5D.

Leading neurite growth throughout the depth of the Puramatrix occurred randomly within the channel with a considerable amount of branching and fasciculation at multiple planes of focus, seen in FIGS. 6A-6C. Conversely, growth in channels deprived of Puramatrix appears limited and aligned along the fibers of the insert at the membrane surface. Antibody labeling in images suggest denser neurite growth along the edges of the channel, as shown by FIG. 12D. Consistent with literature suggesting myelin formation begins after 14 days in vitro, three-dimensional neural cultures showed no presence of myelin after stained with Fluoromyelin™ Red Fluorescent Myelin Stain (Molecular Probes, Eugene, Oreg.) at 7 days in vitro, shown by FIGS. 12E and F. Confocal imaging confirmed β-III tubulin-positive neurites occurred in three dimensions at the beginning, middle, and end of the channel, as demonstrated previously. DAPI-stained nuclei, and S100-positive glial cells occurred throughout the z-stack, as shown by FIGS. 6A-6C.

Using techniques from sample preparation protocols for transmission electron microscopy (TEM) on embedded biological samples, several iterations of post-fixation procedures were tried on the neural constructs before obtaining TEM images with discernible structures. Additional modifications to staining processes may provide structures with higher resolution for clearer visualization. Cross-sectional images from TEM support the evidence shown by fluorescent microscopy. Slices taken within the ganglion and in the neural tract show high density of parallel, highly fasciculated unmyelinated neuritis, as seen in FIGS. 7A and 7B, presence of Schwann cells, as seen in FIG. 7D, and the beginning of Schwann cell encapsulation of neuritis, as seen in FIG. 7C.

Electrophysiological Properties of Neurons in Three-Dimensional Constructs.

To test the functional properties of the three-dimensional neural culture and determine whether it serves as a physiologically active and relevant model of afferent peripheral nervous tissue, intracellular and extracellular electrophysiological experiments were conducted after 7 days in vitro. Using techniques adapted from traditional field potential recordings in acute rodent brain slices, these constructs were studied on an interface chamber permitting the use of a custom rig for extracellular recording. For each experiment, a recording electrode was placed in the ganglion or somatic region of the construct and a stimulating electrode in the channel was inserted along the neurite tract, as shown in FIG. 8A. Following stimulation, a compound action potential (CAP) propagated in a retrograde manner into the somatic region and was recorded as the resulting extracellular potential change in the ganglion for each construct (n=19). The three-dimensional neural constructs supported field recordings for over an hour and consistently displayed coherent population spikes upon stimulation. An example trace of a population response, or CAP, is shown in FIG. 8B Similar to compound action potentials recorded from intact nerves, responses consistently exhibited a short latency to onset followed by a single, cohesive event with a graded nature representing the summed effect of each action potential on recruited axons and corresponding cells. The consistent short envelope and delay of onset of the responses are also characteristic of a CAP and suggest a fast event purely driven by action potentials. As with nerve stimulation, more fibers were recruited with higher stimulus intensities, yielding stronger responses until maximum excitation occurred.

The delay to onset of the response was also increased when the distance between the recording and stimulating electrode was enlarged, as shown in FIGS. 8C and 8D, confirming the ability of the geometrically-confined neural culture to conduct signals at varying distances along its nerve-like tract. On average, responses displayed a delay of onset of 0.82 ms when stimulated proximally or within 1.5 mm from the ganglionic region, as measured from the start of the straight channel. However, when the stimulating electrode was moved 2.25 mm from the ganglion, the distal stimulation yielded delays of onset with an average of 2.88 ms, which are statistically significant, p<0.05, from those observed in proximal stimulation [p=0.02, FIG. 8D]. As seen in fluorescent microscopy and shown in FIGS. 6A-6C and FIGS. 12A and 12B, 7 days of in vitro growth does not allow for neurites to completely fill the channel; a 29.46% decrease in amplitude was exhibited in distal stimulation. Furthermore, by inhibiting Na+ channel activity, action potentials could no longer be generated upon stimulation. Responses from constructs could be completely abolished within 2 minutes of introducing 0.5 μM TTX, confirming the source and biological nature of the responses. Responses before and after TTX wash-in are statistically significant, p=0.029, n=3, shown in FIGS. 8E and F.

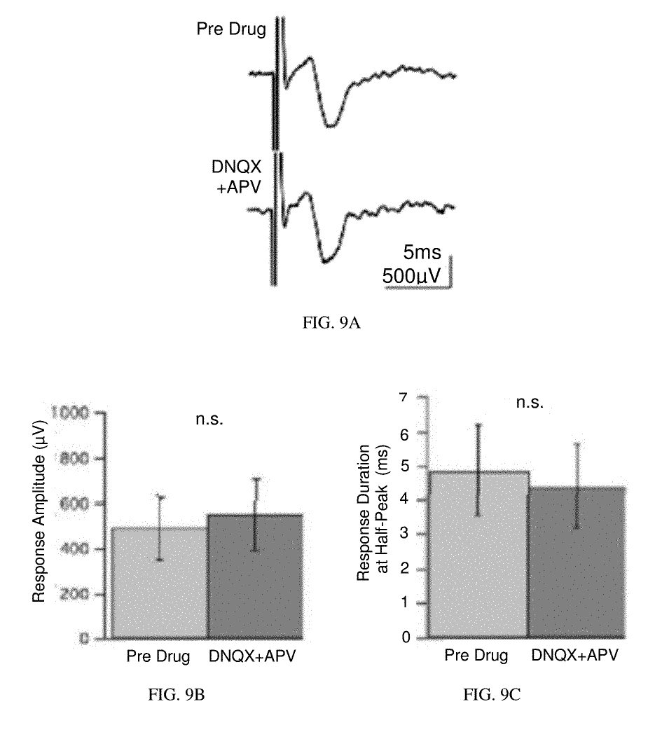

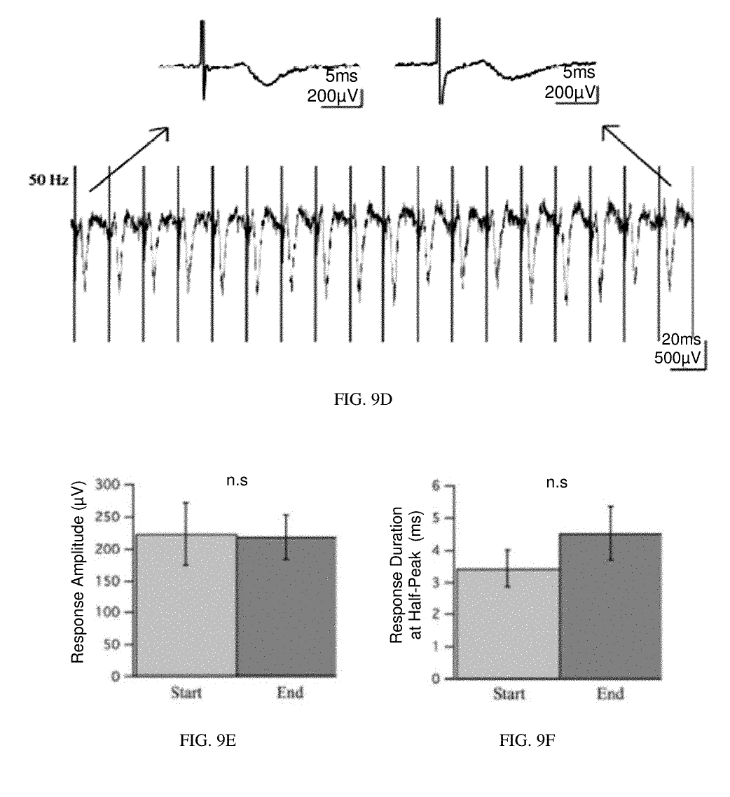

To investigate whether the response was synaptic m nature, glutamate receptor inhibitors DNQX and APV were introduced at 20 and 50 μM respectively to block excitatory synaptic transmission. The experiment lasted for 35 minutes with recordings taken every minute and time points referred to as t1-t35 for simple reference. The drug wash-in occurred 5 minutes into the experiment, t6, and wash-out 20 minutes later, or 25 minutes into the experiment at t26. Responses prior to drug wash-in, t1-t5, were compared to responses recorded 10 minutes into the drug wash-in, t16-t20, allowing ample time for drugs to perfuse and take effect. There was no statistically significant difference observed in the response amplitude or duration before and after wash-in of the drugs, as shown in FIGS. 9A-9C, suggesting there was no synaptic component of the response to block.

A high frequency train of pulses was also induced to assess characteristics of the response. When 20 pulses at 50 Hz were applied to the cultures, the population spikes maintained a consistent delay of onset, envelope, and amplitude, suggesting a strong response capable of repeatedly firing with no depression or facilitation caused by synaptic input. Response amplitude and duration at half-peak before and after high frequency stimulation were not statistically significant, as shown by FIGS. 9D-9F.

Intracellular recordings were also employed, enabling whole-cell patch clamp access inside the three-dimensional neural constructs for over an hour. Modified techniques from whole-cell patch clamping in acute rodent brain slices allowed voltage and current clamp recordings. The Puramatrix gel is more adhesive than native brain tissue and the dense DRG explant contains connective tissue. As with field recordings, these features made movement, replacement, and continued use of the electrodes difficult. Cells within the DRG were densely populated, had less contrast, and were harder to visualize than more sparsely distributed cells in brain slice neuropil that are normally surrounded by features with different diffractive indices. Through repeated visualization in multiple focal planes, positive pressure while navigating through gel, and a tilted electrode approach angle successful whole-cell patch clamp recordings were possible, as shown in FIGS. 10A-10F.

A bipolar stimulating electrode was placed in the neurite tract within the channel and recordings were taken from cells in the somatic region of the construct, shown in FIGS. 10A and 10B. Cells supported electrically-evoked action potentials driven from neurite extensions in the channel, shown by FIG. 10C. As is characteristic of responses lacking synaptic input, intracellular responses had fast rise times, averaging 2 ms baseline-to-peak, with distinct, nongraded onsets as shown in FIG. 10D. There was no rise in potential leading to threshold prior to the onset of the response, as seen in FIG. 10D, nor were there any smaller, graded events following the response, as seen in FIG. 10C, yielding no evidence of synaptic input. Moreover, if synaptic activity were contributing to the onset of the response, threshold for the initiation of action potentials would be harder to reach under hyperpolarization. However, the cells were still able to support action potentials when hyperpolarized from resting membrane potential (RMP) to −100 mV, 1.95× less than the RMP on average, and displayed responses no different than when at RMP. Furthermore, spontaneous activity caused by synaptic activation was not observed in baseline recordings under voltage or current clamp, as shown in FIGS. 10E and 10F.

### Example 2. Growth of Neural Tissues with Schwann Cells in Hydrogel Construct and Assessment of Myelination and Demyelination (Non-Phophetic)

Successful axonal regeneration in the peripheral nervous system (PNS) is dependent upon properly targeting neuronal growth towards a chosen location and upon forming functional synapses for signal propagation. Schwann cells (SCs) native to the PNS play a major role in this process. SCs wrap developing axons in myelin and produce extracellular matrix (ECM) components, cell adhesion molecules, and neurotrophic factors. These events rely on a complex network of signals, including SC-to-neuron, SC-to-SC, and SC-to-ECM communications, from the local microenvironment. Experiments containing SC/Neuron co-cultures provide insight into these processes, leading to new clinical approaches to nervous system ailments.

Primary neurons and SCs have been previously co-cultured in two-dimensional and three-dimensional systems in order to study the mechanisms involved in SC/neuron incorporation. It has been demonstrated that SCs play an important role in orienting developing axons toward their desired targets, leading to functional re-innervation in these models regardless of the number of dimensions. However, many properties involved in SC/neuron incorporation, such as morphology and gene expression, are dramatically affected by system architecture. Three-dimensional systems offer a more accurate representation of the structure and function of the neuronal microenvironment, as well as a better understanding of cell-cell and cell-ECM mechanisms. It has been shown that resting potential, action potential propagation and the function of voltage-gated channels are significantly different in two-dimensional as compared to three-dimensional models. Although the importance of utilizing three-dimensional biomimetic nervous system microenvironments has been demonstrated, few studies investigate SC/neuron interactions in a co-culture and their impact on myelin formation.

Here, the facile and rapid technique described above is employed, using a digital micromirror device (DMD) incorporated with a simple microscope objective to photopattern desired three-dimensional hydrogels. DMDs are capable of structural and molecular three-dimensional micropatterning. This in vitro model provides a setting to mimic the support and three-dimensional architecture of the ECM, with the ability to introduce immobilized or soluble chemical biomolecules, mechanical cues, and drugs independently to evaluate the effects of each on neuronal behavior. This system provides a platform to three-dimensionally co-culture different cell types in one specimen in order to study them in a more biomimetic environment. This approach was used to photomicropattern functionalized Dextran and encapsulate DRGs and SCs in a three-dimensional co-culture system in conditions closer to their natural environment and investigate factors which lead to the formation of myelin.

### A. Materials & Methods

Fabrication of Dual Hydrogel System.

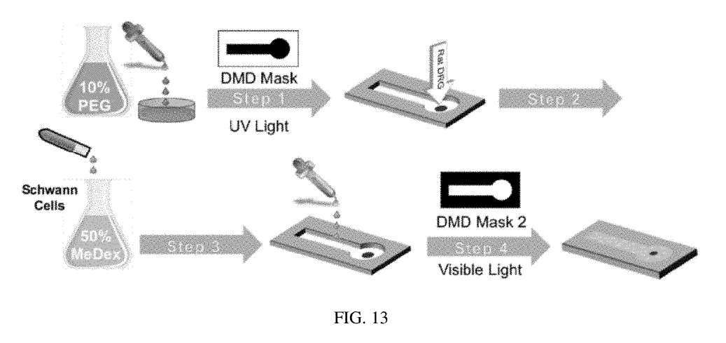

The dual hydrogel culture system was fabricated using a digital projection photolithography, as described above. A schematic of the process is seen in FIG. 13. In brief, a photolithography apparatus comprised of a collimated UV light source (OmniCure 1000 with 320-500 nm filter, EXFO, Quebec, Canada) and a visible light source (SOLA light engine with 375-650 run filter, Lumencor, Oreg., USA), a digital micromirror device (DMD) (Discovery™ 3000, Texas Instruments, Dallas, Tex.) as a dynamic photomask and a 2× Plan Fluor objective lens (Nikon Instruments, Tokyo, Japan) were utilized to irradiate the photocurable hydrogel solution that was contained in a permeable cell culture insert with 0.4 μm pore size. The inserts were either collagen-coated PTFE Transwell® Permeable Support or Transwell™ Clear Polyester Membrane Inserts (Corning Inc., Corning, N.Y., USA) to investigate the influence of collagenated substrates on SC/neuron incorporation. The dual hydrogel system consists of two compartments: a cell permissive section that contains neurons and a cell restrictive section that acts as a hydrogel mold. In order to make the cell restrictive section, a solution of 10% (w/v) PEG-diacrylate (Mn 1000; Polysciences Inc., Warrington, Pa.) and 0.5% (w/v) Irgacure 2959 in PBS was irradiated with 85 mW/cm2 UV light as measured by a radiometer (306 UV Powermeter, Optical Associates, San Jose, Calif.), for 38 seconds to make a PEG micromold, shown in FIG. 13. The cell culture insert was treated with filtered Rain-X® Original Glass Treatment (RainX, Houston, Tex.) prior to addition of the gel solution in order to avoid meniscus behavior. 0.5 ml of solution was added to each 6-well plate insert. Addition of 0.5 ml solution results in a gel thickness of 480 μm. Hydrogel constructs were washed in DPBS with 1% antibiotic-antimycotic additive to inhibit contamination.

Dextran Synthesis and Characterization and Gel Composition.

Dextran (MW=70 kDa) was grafted by Glycidyl methacrylate (GMA) based on a published protocol. Initially, I g dextran was weighed and added to 9 ml dimethylsulfoxide (DMSO) under nitrogen. 0.2 g 4-dimethylaminopyridine (DMAP) was dissolved in 1 ml of DMSO. Subsequently, the DMAP solution was added dropwise to the dextran solution followed by addition of 232 μL GMA under nitrogen. The final solution was stirred for 48 hours at room temperature. In order to quench the reaction after 48 hours, 280 μL 37% hydrochloric acid (HCl) was added to the solution, and the resulting product was dialyzed against deionized water for about three days and lyophilized for about two days. The resulted product was glycidyl methacrylate-dextran (MeDex), and the addition of methacrylate groups to dextran was confirmed using ¹H NMR [(D₂0) δ 6.1-5.7 (m, 2H, CH₂), δ 5.2 (m, IH, CH), δ 4.9 (m, IH, CH), δ 1.9 (s, 3H, CH3)) with substitution degree of 42%. A gel composition of MeDex 50% (w/v), Arg 0.1% of MeDex (w/w), RF 0.001% of MeDex (w/w), TEMED 0.2% of the final solution (v/v) was prepared.

Primary Tissue Culture in the Dual Hydrogel System.

As the first step of the co-culture, the primary tissue culture was performed. The PEG constructs were prepared and immersed in the adhesion media and incubated (37° C., 5% C0₂) overnight prior to the tissue culture. The adhesion media was comprised of Neurobasal medium supplemented with B27 (2% v/v), L-glutamine (0.25% v/v), nerve growth factor (NGF) (0.02 μg/ml), fetal bovine serum (FBS) (10% v/v/) and penicillin/streptomycin (1% v/v) (all from Life Technologies, CA). The constructs were then cultured with Long Evans rat embryo dorsal root ganglion (DRG) tissue, in keeping with the guidelines of the Institutional Animal Care and Use Committee. The DRGs were isolated from embryonic day 15 rat embryos and trimmed prior to the culture. A single DRG explant was placed in each construct. The DRGs were then incubated in fresh adhesion media overnight to allow the tissue to adhere to the insert

Schwann Cell Culture.

An SC cell line (ScienCell Research Laboratories, CA) isolated from neonatal rat sciatic nerves was purchased. The cryopreserved vial with >5×105 cells/ml was thawed in a 37° C. water bath. The contents of the vial were then gently re-suspended and dispensed into the equilibrated poly-L-lysine-coated culture vessel to encourage cell attachment with a seeding density of 2:10,000 cells/cm². The culture was not disturbed for at least 16 hours afterwards. To remove the residual DMSO and unattached cells, the culture medium was changed after 24 hours initially and every other day thereafter. The culture medium was composed of SC medium with FBS (5% v/v), penicillin/streptomycin (1% v/v) and SC medium supplement (1% v/v) (all from ScienCell Research Laboratories, CA). The culture was passaged every time it reached 90% confluence and was not used after the third passage.

SC Encapsulation and Incorporation in the Dual Hydrogel System.

The SCs were dispersed in 50% MeDex solution in SC medium as described above to reach a cell count of 20×10⁶ cell/mL. In order to achieve an evenly distributed single cell solution, the gel mixture was pipetted up and down vigorously. The adhesion medium was aspirated from the channels gently to avoid disturbing the adhered DRGs and 2 μL of the MeDex single cell solution was added to each PEG micromold. A negative photomask was loaded on the DMD and the gel solution in the channel was crosslinked with 85 mW/cm² visible light as measured by a radiometer (306 UV Powermeter, Optical Associates, San Jose, Calif.) after 30 seconds of irradiation using a visible light source (SOLA light engine with 375-650 nm filter, Lumencor, Oreg., USA). The constructs were gently washed using the wash buffer described above three times.

Media Regimen for the DRG/SC Co-Culture in 3D Hydrogel System.

In order to understand the influence of various media regimens on the behavior of DRGs and SCs in a three-dimensional co-culture, two different culture systems were applied. The culture systems are described in Table 2. Culture System 1 has two phases where Media 1 (10 days) and 2 (15 days) are applied in that order. This media regimen has been previously used to promote growth and neurite extension, as well as encouraging endogenic SCs of the DRG bulk to incorporate in myelination process. Culture system 2 only applies Medium 2, which is specialized to induce myelin. The media were changed every other day for each specimen in each experimental group.

TABLE 2
Culture Media Systems

| | | |
|---|---|---|
| Component | Media 1 | Media 2 |

| | | |
|---|---|---|
| Basal Eagle's Medium | yes | yes |

| Glutamax | 1% | v/v | 1% | v/v |
|---|---|---|---|---|
| ITS supplement | 1% | v/v | 1% | v/v |
| BSA | 0.2% | w/v | none | |
| D-glucose | 0.4% | w/v | 0.4% | w/v |
| 100 μg/ml NGF | 10 | μL | 10 | μL |
| Penicillin/Streptomycin | 1% | v/v | 1% | v/v |

| | | | |
|---|---|---|---|
| FBS | none | 15% | v/v |

| | | | | |
|---|---|---|---|---|
| L-ascorbic acid | 0.004% | w/v | 0.004% | w/v |
| Culture System 1 | 10 | days | 15 | days |

| | | | |
|---|---|---|---|
| Culture System 2 | none | 25 | days |

Immunohistochemistry.

To evaluate neurite growth and myelin formation, immunohistochemistry techniques were utilized. Initially, the tissue was fixed with 4% paraformaldehyde (PFA) for 2 hours at 37° C. followed by three washing steps prior to each staining procedure. All of the reagents were provided from AbCam, Cambridge, Mass., unless otherwise is stated.

Neurites were labeled with mouse monoclonal [2G10] neuron-specific β-III tubulin primary antibody and Cy3.5 conjugated goat anti-mouse immunoglobulin G (H+L) secondary antibody (AbCam, Cambridge, Mass.). The labeling steps were completed in 2% bovine serum albumin (BSA) and 0.1% saponin in PBS, overnight at 4° C. and every step was followed by three washing steps with PBS.

To assess myelin formation, constructs were labeled for three myelin proteins: Myelin Basic Protein (MBP), Protein Zero (PO) and Myelin Associated Glycoprotein (MAG). Primary antibody chicken polyclonal anti-Myelin Basic Protein, mouse monoclonal anti-Myelin Associated Glycoprotein and rabbit polyclonal Anti-Myelin Protein Zero antibody were utilized. The stains were diluted in 2% BSA/PBS solution with a concentration of 1:500. The constructs were immersed in 5% goat serum at room temperature for 30 minutes in order to avoid any nonspecific protein binding The constructs were stored at 4° C. overnight in primary antibody solution and were washed three times with PBS. After three washing cycles, the hydrogel systems were incubated at 4° C. in the secondary antibody solution. The secondary solution was prepared as follows: 1:500 antibody solution in 2% BSA solution Goat Anti-Chicken IgY H&L, Goat Anti-Mouse IgG H&L and Goat Anti-Rabbit IgG H&L, respectively.

Image Processing, Neurite Growth, and Myelin Formation.

The volume of growth into the three-dimensional hydrogel was measured utilizing a confocal microscope (Nikon AI, Tokyo, Japan). Because of the entangled and dense neurite outgrowth in the model, it is difficult to count the number of individual neurons as it extends along the length. Therefore, in order to measure the growth of the system in three dimensions, it is optimal to take the volume of cellular mass in the dual hydrogel culture systems. Each sample was imaged in three dimensions with optical slices no greater than an 11 μm depth with an average of 20 slices per sample, a resolution of 1024×1024 pixels and with a 10× objective. Pre-processing steps including thresholding and transformation into a binary representation were applied uniformly across all images. Data analysis was performed using ImageJ and a custom algorithm in Matlab (Mathworks, Natick, Mass.). Neurite growth was quantified using pixel counts of the threshold slices throughout the depth of the gel. After 25 days myelin was dense and entwined, and same image processing procedures were utilized in order to evaluate the volume of myelin throughout the depth. This process allows measurement of the volume throughout the depth, considering the three-dimensional nature of the cultures. Because the size of the constructs was too large to be imaged at once, a large-image z-stack was taken (1×5) for both imaging processes above. For demonstration pictures, samples were imaged in three dimensions with an optical slice not greater than 11 μm in depth with an average of 20 slices per sample, a resolution of 1024×1024 pixels, and a 20× objective. A maximum projection acquisition was used in order to form two-dimensional images of the total growth. For the volume of growth, the same procedure was utilized and the three-dimensional volume acquisition was used in order to confirm that the growth and myelination occurs throughout the depth.

Transmission Electron Microscopy.

TEM was utilized to investigate the nanoscale structure of neuronal processes, SCs, and their spatial crosstalk, distribution, and morphology in the hydrogel cultures. All of the reagents used for this procedure were provided from Electron Microscopy Sciences, Hatfield, Pa. unless otherwise stated. The hydrogel constructs were fixed after submerging in 4% PFA solution for about two hours at 37° C. The samples were then washed three times in 15-minute intervals with PBS. The post-fixation steps included staining with 1% osmium tetroxide (OsO₄) in 100 mM phosphate acetate for about 2 hours followed by four washing steps with PBS. The tissue was then stained with 2% aqueous uranyl acetate for about 30 minutes at room temperature in the dark. The procedure was followed by dehydration steps, including immersing the samples in 50% and 70% ethanol for 10 minutes each, then in 95% ethanol overnight. The samples were then soaked in 100% ethanol that was filtered with Molecular Sieves, 4 Å (Sigma-Aldrich, St. Louis, Mo.) for two 30-minute intervals. The constructs were cut to maintain only the regions of interest, followed by resin embedment. An infiltration step was performed using a 1:1 propylene oxide-spurr resin for 45 minutes. The samples were then embedded in 100% spur resin at 70° C. for about 48 hours in order to allow the resin polymerization to complete.

Embedded samples were trimmed and sliced with thicknesses varying from 80 nm to 100 nm using a Reichert Ultracut S ultratome (Leica Microsystems, Buffalo Grove, Ill.) and Ultra 45° diamond knife (Diatome, Fort Washington, Pa.). The slices were loaded on copper grids (Formvar carbon-coated, 200 mesh), and the grids were floated on droplets of 2% uranyl acetate for about 20 minutes and rinsed by floating on deionized water droplets three times in 1-minute intervals. After mounting the grids on a single-tilted stage, they were imaged using a FEI Tecnai G2 F30 Twin transmission electron microscope (FEI, Hillsboro, Oreg.) with an accelerator voltage of 100-200 kV. linages were taken at 3,000×-20,000× magnifications with 4000×4000 pixel resolution.

### B. Results

Three-Dimensional Dual Hydrogel System and DRG/SC Co-Culture.

The present disclosure provides a three-dimensional model to investigate the use of a dual hydrogel platform for co-culture applications and a three-dimensional hydrogel system using a DMD as a dynamic photolithography tool. Utilizing this model, the influence of mechanical stimuli and chemical cues, including repulsive and attractive biomolecules, on neuronal outgrowth in vitro was investigated. This model mimics the three-dimensional structure of the ECM and translates neuronal microenvironment more accurately. The ability of this system to handle two cell types in single culture and to investigate the cells behavior was evaluated. SCs and neurons were co-cultured to examine the myelination processes in conditions closer to their natural environment. This model allows myelin formation as a result of SC-neuron co-cultures in three dimensions. The methodology for the dual hydrogel system is depicted in FIG. 13.

The Influence of Collagen on Neurite Growth in Three-Dimensional Co-Cultures.

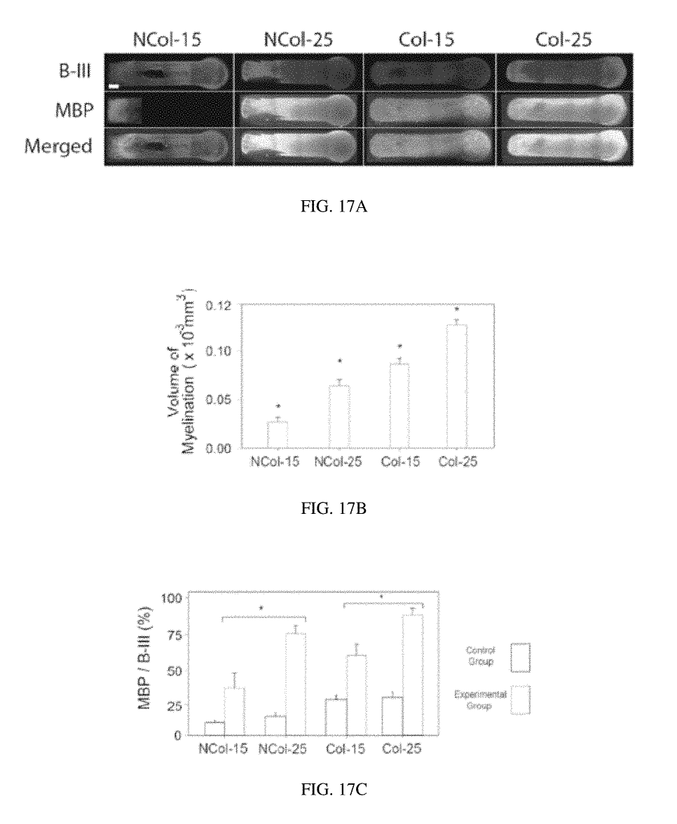

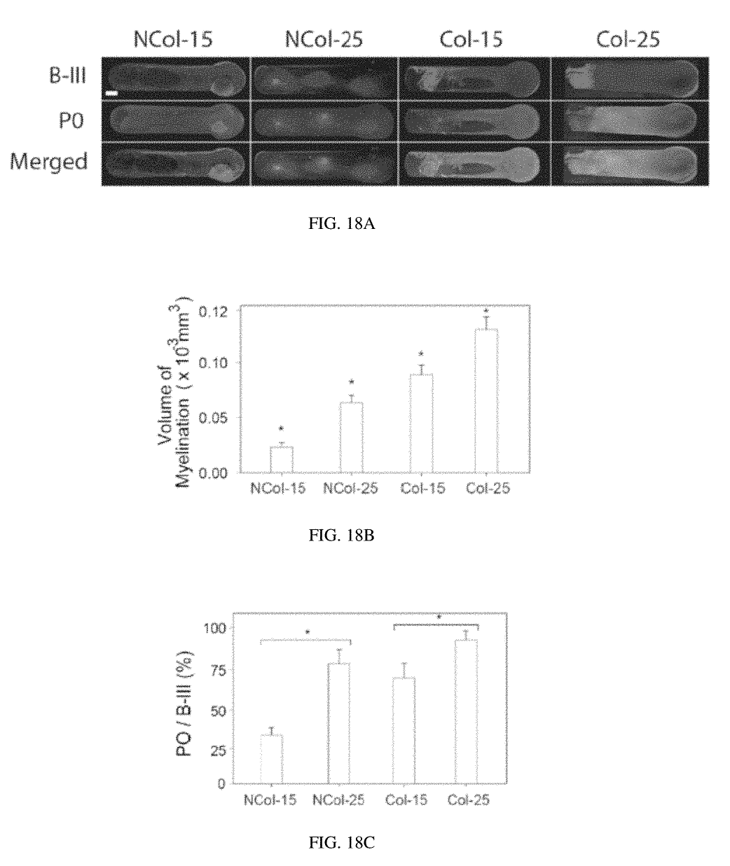

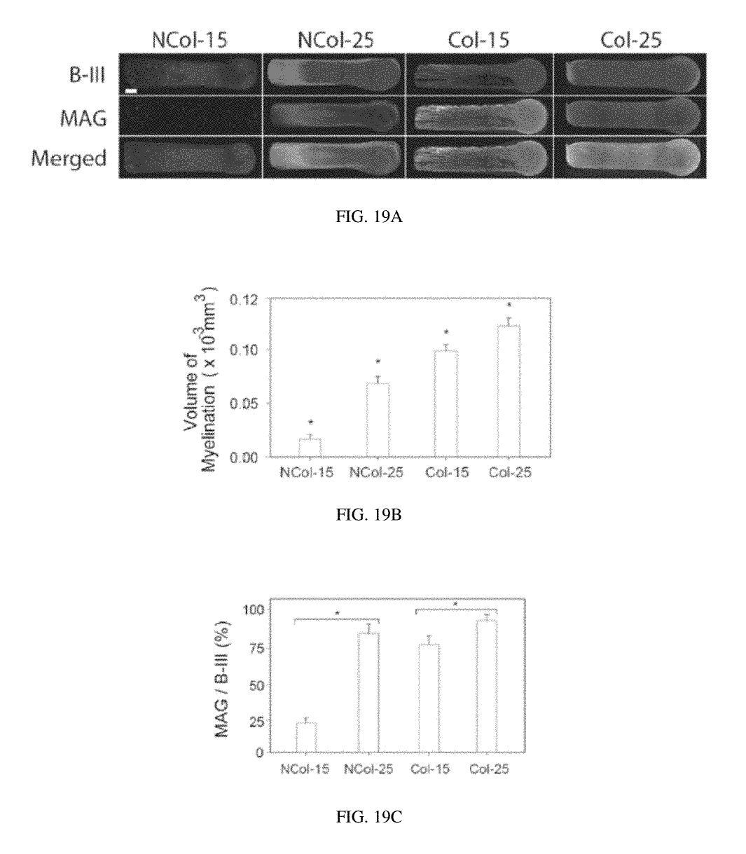

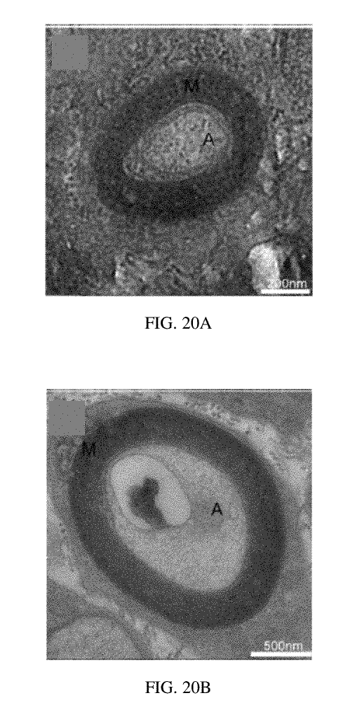

The formation of three-dimensional cultures within hydrogels formed in permeable inserts with or without a collagen coating was demonstrated. The growth in both cultures was robust, fasciculated, and aligned. This characteristic differentiates this system from previously developed in vitro models, as the growth is directed within a channel. Although the growth is highly dense after 25 days, it is mostly contained in the cell-permissive section of the three-dimensional hydrogel system. The β-III tubulin positive neuronal filaments are depicted in FIG. 17A, FIG. 18A, and FIG. 19A. There is a significantly higher volume of neuronal outgrowth in the cultures with collagen compared to the cultures without collagen (n=15-18 constructs). The amount of growth was not substantially different between the two media regimens.

Myelin Development in Three-Dimensional Co-Culture Model in Dual Hydrogel System.

The presently-disclosed co-culture system promotes myelin formation in three dimensions. Immunohistochemistry and TEM were utilized in order to prove the formation of myelin. The cultures were stained with three antibodies: MBP, MAG and PO. The constructs were positive for MAG, MB1P and PO, confirming the formation of compact and non-compact myelin. FIG. 17B and FIG. 18B both show neurofilaments stained for β-III tubulin and the merged images that confirm the formation of MBP and PO segments along the axonal extensions; FIG. 17B shows MBP-positive mature myelin sheath; and FIG. 18B shows PO-positive mature myelin sheath.

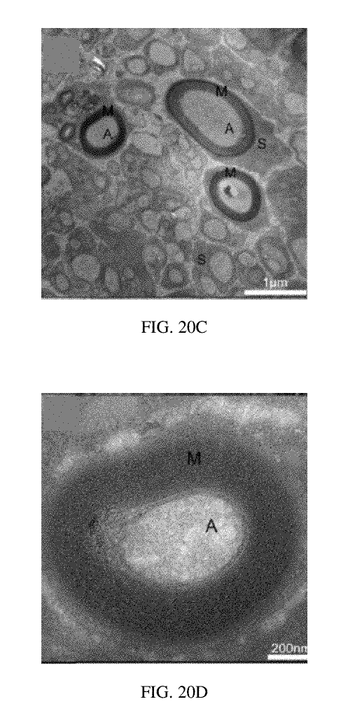

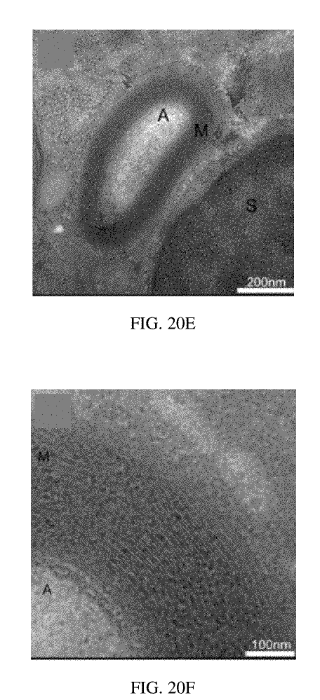

As described above, all images were taken through z-stack acquisition. Confocal imaging confirmed that neurite growth occurred in three dimensions throughout the channel. The depth of growth and myelination for these constructs was 88±15 μm. TEM images confirmed myelin formation, seen in FIGS. 20A-20F. Slices taken in the neural tract show a high density of parallel, highly fasciculated, and myelinated neurites, presence of Schwann cells, and Schwann cell encapsulation of neurites. Myelin segments were consistently identified in TEM images, confirming compact myelin formation. These findings demonstrate that this three-dimensional in vitro model enables SCs to form mature myelin layers around neurites.

The Effect of Ascorbic Acid (AA) on Myelin Formation in Three Dimensions.

Two media regimens were used for the cultures. For NCol-25 and Col-25, 25 days of media containing AA resulted in a considerable increase in the amount of myelin. The amount of myelin demonstrates the ability of the culture to form myelin sheaths, regardless of the amount of neuronal growth. The ratio of myelin to neuronal growth was measured, showing that the percentage of myelin in the constructs increases with longer exposures to AA. This was confirmed through three immunohistochemistry antibody stains for MBP, MAG, and PO, thus demonstrating that this is accurate for both compact and non-compact myelin.

The Impact of Collagen on Myelin Development.

The influence of collagen I and III on compact and non-compact myelin development was evaluated in the system. The myelin proteins followed similar trends, as shown in FIGS. 17A-17C, FIGS. 18A-18C, and FIGS. 19A-19C. The addition of collagen increased the amount of myelin formation in the system. The ratio of myelin to neurite growth was similar for Col-15 and NCol-25. This demonstrates that increased quantities of myelin in Col-15 compared to NCol-25 are due to increases in the amount of neuronal growth. The efficiency of the two systems in developing myelin is dependent on AA exposure. FIGS. 16A and 16B shows that collagen augments neuronal growth drastically. NCol-15 shows that in the absence of collagen and with a shorter exposure to AA, the least myelin forms.

### C. Discussion

The myelin sheath is a specialized cell membrane with a multi-lamellar spiral structure that surrounds the axon and reduces nervous system capacitance. Well-myelinated nerves are completely surrounded by myelin sheaths except for small, periodic gaps known as nodes of Ranvier that are exposed to the extracellular environment. Myelin exists in two forms: compact and non-compact. The compact myelin ultrastructure consists of a spiraled cellular sheath that lacks cytoplasm as well as extracellular spaces but does contain two plasma membranes. Non-compact myelin is the channel-like segment of myelin and is non-condensed and is made of Schmidt-Lanterman incisures, periodic interruptions in the myelin layer, and paranodal regions.

Compact myelin and non-compact myelin each contain various proteins, such as Myelin Basic Protein (MBP), which is an essential component of CNS and PNS compact myelin. MBP is located on the cytoplasmic surface of the myelin sheath and is extremely charged. Another vital myelin protein in the PNS is PO, which is a transmembrane glycoprotein that affects cell adhesion, maintains the main dense line of PNS compact myelin, and plays an important role in keeping the space between compact myelin consistent. One of the major components of non-compact myelin is Myelin Associated Glycoprotein (MAG), which does not exist on the outer layer of myelin but is present in the inner layer. It is in contact with the axon, connecting it to compact myelin. These three proteins are essential for myelin formation and maintenance and have been widely utilized to detect myelin in cultures.

A co-culture system of SCs and neurons, derived from either a primary tissue source or a cell line, may accurately portray the events of the native PNS and the complex myelin architecture. PC 12 cell lines and SCs have been previously used with the aim of establishing motor neuron/SC co-culture models in order to study motor neuron diseases. An in vitro model of sensory neurons and SCs was previously used in order to understand the mechanisms behind myelination. Many previous studies employ DRGs, as they are well-studied and are recognized as strong in vitro models that employ the development of neuron/SC co-cultures to evaluate myelination processes in the PNS.

These previous in vitro co-culture models have been performed mostly in two-dimensional cell cultures and three-dimensional tissue slices. There are few studies that investigate the incorporation of neuron/SC co-culture and their influence on myelin formation in three-dimensional cultures.

To design a three-dimensional biomimetic polymer model in order to study myelination in neuron/SC co-cultures, photomicropatterning settings were utilized. Photopatterning has been used to study the nervous system because it allows proper translation of the biomimetic neuronal microenvironment in three dimensions. The dynamic mask projection photolithography apparatus that was utilized in this study provided an easy fabrication technique for the purpose of producing micropatterned hydrogels. These hydrogels were created on permeable cell culture inserts that provide the basis for the neural regeneration experiments.

In order to generate these constructs, a DMD device was utilized to create a dynamic photomask. This mask was used with irradiated PEG solution to create the mold into which DRGs were initially adhered, which was followed by the addition of a photocurable single cell MeDex solution. A negative dynamic photomask was utilized to encapsulate SCs in three dimensions and to incorporate them with the DRGs. Utilizing visible light with short (30 seconds) exposure lengths is the most practical for hydrogel formation and cellular encapsulation in order to decrease cytotoxicity, and these procedures were utilized for this design. This model provided a long-term (25 days) in vitro platform that ensures the survival of neurons, their elongation, and their myelination in three-dimensional environment.

These models used two different cell culture media, as described in Table 2. Medium 1 is composed of factors that have been well-characterized and are known to support DRG and SC growth. This medium contains BSA, which has been shown to support migration of SCs. However, this system is not specialized to promote myelin formation. Medium 2 contains FBS in conjunction with ascorbic acid, which has been demonstrated to promote myelination in two-dimensional cultures. Previous studies of SCs in the presence of neurons show that they are able to create a complete ECM with a basal lamina and collagen fibrils in vitro. SC/DRG co-cultures have shown that that ascorbic acid may promote SCs to generate myelin by enabling them to form a basal lamina. Medium 2 also contain ITS (insulin, transferrin and selenium), which has been shown to promote myelination in rat cell lines.

Laminin was utilized in every experimental group, as it has been demonstrated in neuron/SC co-cultures to be necessary for myelination. In vivo, the absence of laminin has been shown to lead to peripheral neuropathy in both mice and humans. Mutant mice that are deficient in laminin will have disruption of the endoneurium basal lamina, which subsequently reduces nerve conduction velocity.

The systems presently disclosed also examine the effects of the presence of collagen on neuronal growth in this three-dimensional model through the use of collagen-coated substrates. Type I and Type III collagen was utilized for these studies. Type III collagen binds to and activates an adhesion g-protein coupled receptor on Schwann Cells, Gpr56, which may lead to the activation of Gpr125 to initiate myelination. Type I and Type III collagen are key components of the epineurium, which is the outermost layer of dense tissue that supports and surrounds peripheral nerves and myelin.

To investigate the ability of neuronal cells to form myelin in a three dimensional model, the influence of two different media and the impact of collagen was evaluated. The four culture systems are differentiated by the presence of collagen and the media regimen the co-cultures were exposed to. Two media regimens were utilized. One regimen comprised Medium 1 for 10 days and then Medium 2 for 15 days (Culture System 1); in the second regimen, the cells were exposed to only Medium 2 for 25 days (Culture System 2). Table 3 describes the groups. In order to determine whether the myelination was influenced by exogenic SCs, the above experiments were performed without the addition of encapsulated SCs to the dual hydrogel system, while holding all other variables constant.

TABLE 3
Culture Groups

| Culture Name | Type I & Type III Collagen | Media Regimen |
|---|---|---|
| NCol-15 | No | Media 1 (10 days); |
| | | Media 2 (15 days) |
| NCol-25 | No | Media 2 (25 days) |
| Col-15 | Yes | Media 1 (10 days); |
| | | Media 2 (15 days) |
| Col-25 | Yes | Media 2 (25 days) |

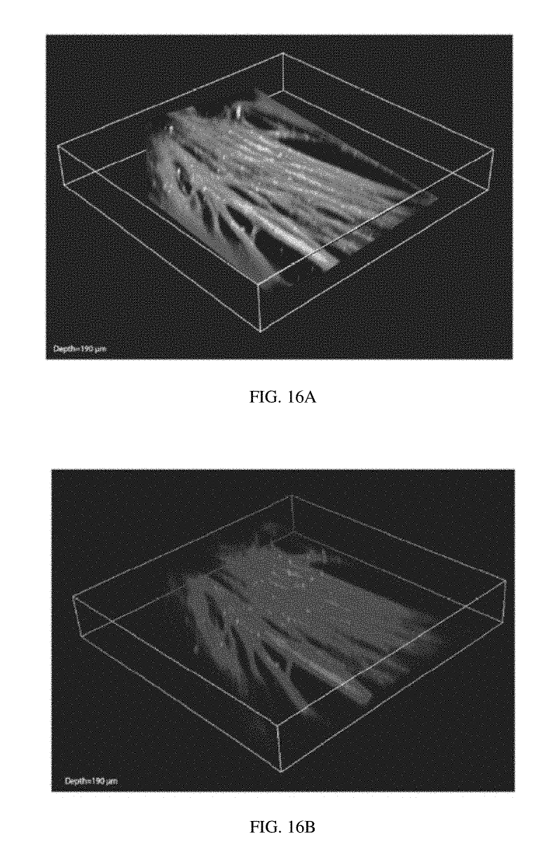

The formation of myelin was confirmed using immunohistochemistry and confocal imaging and was further validated by TEM. Two-dimensional images of 20× magnification show the formation of myelin segments that wrap around the neuronal projections in MBP/β-III tubulin-positive cultures, shown in FIG. 15. The three-dimensional development of myelin, stained for both MBP and MAG due to the formation of compact and non-compact myelin, is depicted in FIGS. 16A and 16B. TEM images also confirmed the occurrence and abundance of mature myelin layers in all of the experimental groups, shown in FIGS. 20A-20D. A magnified image of myelin layers is depicted in FIG. 20F. FIG. 20E shows that after 25 days in culture, SCs had formed myelin sheaths around many of the neurites, and some SCs had begun to roll cytoplasmic layers around the nerve fibers. This image demonstrates that the amount of myelin is significant and that the cultures can be utilized for long-term studies, including long-lasting drug evaluations in three dimensions. FIGS. 20A-20F also shows the high density of aligned, highly fasciculated neurons in the culture.

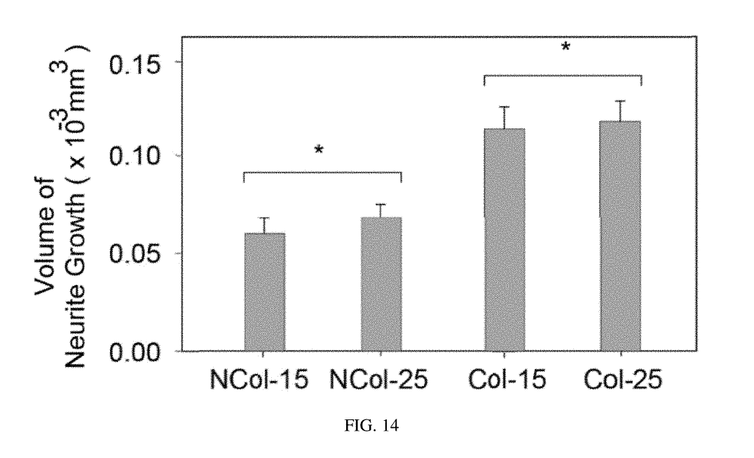

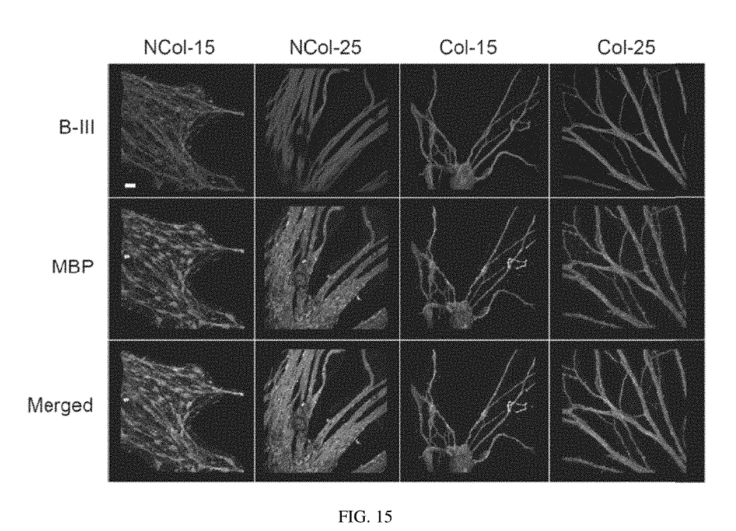

The first set of analyses performed quantified the amount of neuronal growth in each of the four culture systems in three dimensions, as described in FIG. 14. It is well-established that collagen and their receptors promote neurite outgrowth. The data presented here demonstrate that there is significantly more neuronal growth in the two systems where collagen is present. However, there was no significant impact on growth due to the media regimen that was utilized, demonstrating that it had little impact on the amount of neurite extension after 25 days in the contained system.

The amount of myelin was measured by two different approaches. The first approach was to look at myelination as an independent variable and scrutinize the total amount of myelination, regardless of the amount of neuronal development in the system. The second approach was a calculation of the ratio of myelin to neurite extension and normalizing the amount of the myelin development. This provides an understanding of the myelination efficiency and describes the percentage of neuronal projections with myelin sheaths wrapped around them. Stains for MBP, MAG and PO were utilized to investigate the amount of myelin produced by the four experimental groups.

FIG. 17C describes the percentage of myelin formed in the culture systems. An MBP antibody was utilized for these data. While all four samples were positive for MBP after 25 days of culture, there were significant differences between the groups. MBP is a protein that exists in compact myelin, and its expression in the culture verifies the formation of compacted membrane segments of mature myelin sheath. Increased myelination occurs in these systems when there is increased AA exposure. These results were achieved in a three-dimensional in vitro model that mimics the environment of the nervous system more closely than typical two-dimensional cultures or tissue sections. The data indicate that there is a significant increase in the ratio of myelin to neuronal outgrowth in these systems when exposed to myelination media for 25 days. The media regimens result in increased myelination when the cultures are in the presence of collagen for the same exposure length.

Based on these data, two factors play a role in these cultures: the presence of collagen and a longer AA exposure. The constructs lacking both of these factors (NCol-15) are the least myelinated. The percentage of myelin to neuronal growth for the cultures showed that the same AA exposure had a similar effect, regardless of the number of neurons that had been produced. However, FIG. 17B shows that when both factors are present in the experiment (Col-25), a synergistic response is observed, resulting in a significant increase in myelin magnitude. Maximum projections of z-stack planes are included to support these data.

In order to confirm that exogenous SCs significantly alter myelination, a control group with no additional SCs was utilized. The data in FIG. 17C show that every experimental group had a significant increase in myelination versus its corresponding control, demonstrating that exogenous SCs had a large impact on the system. The results show that collagen significantly increases myelination in the control groups, but AA exposure duration has a lesser impact.

Myelination was measured in the three-dimensional cultures using PO protein antibody. 70% of the total proteins in PNS myelin consist of PO, and a lack of this protein would verify a lack of non-compact myelin. The ratio of PO expression to β-III tubulin-positive neurofilaments was evaluated. The results shown in FIG. 18C demonstrate that NCol-15 presents the least amount of PO out of all the cultures. The percentage of PO expression is substantially higher in cultures in the presence of AA for 25 days, which agrees with the results from MBP staining that show the most expression of MBP in the Col-25 group. This is interesting, as PO and MBP are both signature proteins of compact myelin in PNS but have different responsibilities. PO retains the organized recurrence of both the ECM and cytoplasmic spacing of the myelin membrane while MBP plays a role in cytoplasmic fusion. This value is equivalent for NCol-25 group, showing that the efficiency of the cultures after 25 days of Medium 2 was the same regardless of whether collagen was present in the cultures.

FIG. 18B shows the amount of myelin in the cultures labeled with PO. Col-25 shows the maximum amount of compact myelin PO development, regardless of the amount of the neuronal growth. The results show that the samples with collagen in the culture led to more neurons, resulting in a higher amount of myelin. Between the two collagen-containing samples, the exposure to AA increases the amount of PO occurrence. This is maintained even after normalizing the volume of myelin values in collagen-containing samples by calculating the ratio of myelin to volume of the neurofilaments, as shown in FIG. 18C. The images demonstrate that the amount of PO decreased drastically in the constructs with no collagen, NCol-15 and NCol-25. The volume of neuronal growth also decreased, and as a result, the percentage of compact myelin formation did not show any significant variance from the Col-15. In these long term, three-dimensional constructs, the percentage of compact myelin that expressed PO after the culture is exposed to AA for 25 days is not substantially different from the percentage of compact myelin expressing PO in the cultures with collagen in the presence of AA for 15 days. AA is necessary for myelination in serum-containing media for two-dimensional cultures. The duration AA exposure plays an important role in efficiency of the formation of myelin. Collagen I and III support neuronal growth and can aid in initiating the myelination process. The presence of collagen in the system increases the neuronal three-dimensional extension, and as a result, augments the amount of myelin formation in a three-dimensional setting.

A different measure for myelin is MAG, a protein that is abundant in non-compact myelin. The ability of rat DRG/SC co-cultures to form myelin in the three-dimensional construct was evaluated by MAG immunostaining. All of the constructs were MAG-positive and followed the same pattern as PO and MBP. High levels of myelin synthesis were demonstrated by confocal microscopy analysis of MAG, similar to PO and MBP. MAG indicates the Schmidt-Lanterman incisures and paranodes that are characteristics of non-compact myelin. The amount of non-compact myelin, regardless of the volume of neuronal growth, was higher in the Col-25 group in the presence of collagen with longer AA exposure. AA helps the system form basal lamina and encourages myelin formation. The percentage of the MAG-labeled structures is not substantially different between the cultures with the same exposure to AA (Col-25 and N-Col 25). However, the amount of growth substantially decreases when collagen is not added to the system.

The present disclosure discloses a novel, three-dimensional, in vitro co-culture model that allows incorporation of SCs and neurons. A facile high-throughput photolithography method that provided a three-dimensional setting was utilized to replicate neuronal phenomena in controlled microenvironments to introduce mechanical and chemical cues with highly-resolved spatiotemporal precision. Here, the data demonstrates that this co-culture setting provided aligned, highly fasciculated neuronal growth with myelin sheaths nicely wrapped along them. Myelination was confirmed through immunohistochemistry and TEM. Two culture systems were used, and the influence of collagen on neuronal growth and myelination was investigated. This platform provides useful devices, methods, and systems for drug discovery and evaluation.

### Example 3. Calibration and Feasibility of Model (Non-Prophetic and Prophetic)

The drug development pipeline is plagued by unacceptable rates of attrition due in large part to toxicities that are not identified in pre-clinical stages of development. Chemotherapeutics in particular, while clinically effective against a wide array of cancers, are commonly associated with dose-limiting systemic toxicities. In many cases, the peripheral nervous system bears the brunt of these adverse effects, and such toxicity is often only first identified in animal studies or overlooked until clinical trials. Chemotherapy-induced peripheral neuropathy (CIPN) is a common side effect of cancer treatment, causing many patients to alter dose regimens and some to cease treatment altogether due to serious neurotoxic damage. The ability to screen drug candidates for peripheral neurotoxicity in a cellular model would speed the drug discovery process by aiding companies in identifying promising lead compounds before undertaking costly and time-consuming animal studies.

“Organoid-on-a-chip” technologies show tremendous promise as advanced cellular models that can provide medium-throughput and high-content data useful for late-stage drug development, provided that they supply information that is predictive of human physiology or pathology. A number of contract research organizations have seen commercial success providing such assays for various organ systems. However, development of peripheral nerve-on-a-chip assays is lagging. Commonly-used peripheral neural culture preparations are not predictive of clinical toxicity, partially because they typically utilize apoptosis or neurite elongation as measurable endpoints, whereas adult peripheral neurons are fully grown and known to resist apoptosis. Nerve conduction testing and histomorphometry of tissue biopsies are the most clinically-relevant measures of neuropathy. Nevertheless, there are currently no culture models that provide such metrics.

We have developed an innovative sensory-nerve-on-a-chip model by culturing dorsal root ganglia in micropatterned hydrogel constructs to constrain axon growth in a 3D arrangement analogous to peripheral nerve anatomy. Further, electrically-evoked population field potentials resulting from compound action potentials (cAPs) may be recorded reproducibly in these model systems. These early results demonstrate the feasibility of using microengineered neural tissues that are amenable to morphological and physiological measurements analogous to those of clinical tests. We hypothesize that chemotherapy-induced neural toxicity will manifest in these measurements in ways that mimic clinical neuropathology. The goal of this proposal is to demonstrate the feasibility of using the compound action potential waveform as a measure of peripheral neurotoxicity in vitro. To do this, we will apply chemotherapeutic drugs with known peripheral neurotoxicity, measure changes in cAPs, and compare with morphological changes as well as documented clinical pathophysiology. The following Specific Aims will allow us to achieve this goal:
Aim 1: Calibrate nerve-on-a-chip model by quantifying key morphological metrics and correlating with compound action potential (cAP) metrics.
   Quantify cell body size and density, and neurite density, diameter, and % myelinated neurites at three lengths along tract over four weeks in vitro using confocal and transmission electron microscopy.
   Determine consistency of evoked population action potential responses over four weeks.
   Correlate cAP waveforms with morphometric parameters to determine baseline structure-function relationships.
Aim 2: Demonstrate the feasibility of using the cAP waveform to measure toxicity induced by acute application of four chemotherapeutic agents known to cause clinical neuropathy.
   Determine dosages and incubation times of oxaliplatin, paclitaxel, vincristine, and bortezomib appropriate for the nerve-on-a-chip model in a pilot study.
   Measure cAP conduction velocity, amplitude, latency, and integral after drug administration at end points determined in pilot study.
   Quantify morphometric changes and determine correlations with changes in cAP waveforms.

It is widely recognized that current attrition rates of experimental drugs progressing from discovery to clinical use are unacceptably high, driving the cost to bring a single drug to market up to $2.6 B (DiMasi et al 2014). Dose-limiting toxicity that is not discovered during drug development is estimated to be the second-leading cause of post-marketing drug withdrawal, and these late stage failures are generally associated with a lack of reliable screening methods for drug candidate toxicity (Kola & Landis 2004, Li 2004, Schuster et al 2005). Despite this, the most current guidelines from the FDA on in vitro-in vivo correlations (IVIVCs) emphasize the relationship between drug dissolution and bioavailability (Emami 2006); there are no IVIVC guidelines defined for correlating clinical toxicity with toxicity testing in vitro. It is clear that cell-based toxicity screening assays would aid companies in identifying lead compounds with lower toxicity, but in vitro assays that are reliably predictive of clinical toxicology are sadly lacking and desperately needed (Astashkina & Grainger 2014).

Chemotherapeutics are a special class of drugs, since they are cytotoxic by their very nature. Toxic side-effects are therefore unavoidable, and the level of systemic toxicity that is clinically tolerable limits the drug dosage. The nervous system is particularly vulnerable to adverse effects, with neurotoxicity associated with chemotherapy being second in incidence only to hematological toxicity (Malik & Stillman 2008, Windebank & Grisold 2008). The peripheral nerves are especially susceptible, probably owing to being outside of the protective blood-brain barrier and having very long axons reaching far from their cell bodies. Chemotherapy-induced peripheral neuropathy (CIPN) is estimated to occur in 30-40% of patients undergoing treatment, and sensory nerves are affected consistently more severely than motor nerves (Windebank & Grisold 2008). Symptoms range from chronic pain in the extremities, to tingling, lack of sensation or joint position sense, and motor deficits. The National Cancer Institute identified CIPN as one of the most dose-limiting side-effects and the most common reason patients elect to reduce dosage or stop treatment altogether (Moya del Pino 2010). In some cases, the symptoms resolve after cessation of treatment, but most often CIPN is only partially reversible with some symptoms remaining permanently. Unlike hematological toxicity, which can be treated readily, there are currently no standard-of-care clinical treatments for CIPN (Windebank & Grisold 2008).

The classes of chemotherapeutic agents known to pose the greatest risk for peripheral neurotoxicity are platinum derivatives; tubulin-binding compounds, including vinca alkaloids, taxanes, and epothilones; the proteasome inhibitor bortezomib; and thalidomide. These drugs are also the standard of care for the six most common malignancies (Argyriou et al 2012, Cavaletti & Marmiroli 2010, Wang et al 2012). The exact neurotoxic molecular mechanisms leading to the range of symptoms reported are varied and, in some cases, remain unclear. In general, platinum compounds bind DNA and cause apoptosis, while antitubulins disrupt tubulin dynamics including axonal transport (Malik & Stillman 2008); bortezomib is thought to disrupt mRNA transcription and processing in the ganglion, and the mechanism of thalidomide is unknown, though it may involve interactions with the vasculature and/or inflammatory cells (Argyriou et al 2012). The specific presentation and severity of CIPN can be most objectively and reliably diagnosed by nerve conduction tests and/or skin or nerve biopsies (Dyck & Thomas 2005). These measurements are currently only obtainable from safety tests in animals and humans. So, most drug companies simply do not screen specifically for peripheral neurotoxicity until after lead compound identification, even though it is one of the most likely causes of failure in later stages of development.

The use of 3D “organoid-on-a-chip” models is gaining acceptance as the best hope for developing predictive cell-based assays suitable for drug development and toxicity screening (Ghaemmaghami et al 2012, Kimlin et al 2013). However, it is critical that such model systems move beyond 3D versions of conventional cell viability assays to models that truly recapitulate functional aspects of organ physiology that can be evaluated to identify toxicity pathways (Astashkina & Grainger 2014). Such physiological assessment is especially challenging for peripheral neural tissue, where bioelectrical conduction over long distances may arguably be the most relevant physiological endpoint. For this reason, 3D tissue models of peripheral nerve are lagging those of epithelial, metabolic, and tumor tissues, where soluble analytes serve as appropriate metrics. A nerve-on-a-chip model that makes use of clinically-relevant toxicity metrics would be tremendously valuable for pre-clinical drug development by enabling selection of promising lead compounds with lower chances of late-stage failure due to peripheral neurotoxicity. Further, the high-content information provided by such a model would be valuable for investigative toxicology by providing insight into the possible mechanisms of toxicity, thus guiding reformulation. By demonstrating the feasibility of our model system, we expect to strongly position ourselves as a commercial front-runner, with first-to-market technology in predictive screening for peripheral neurotoxicity

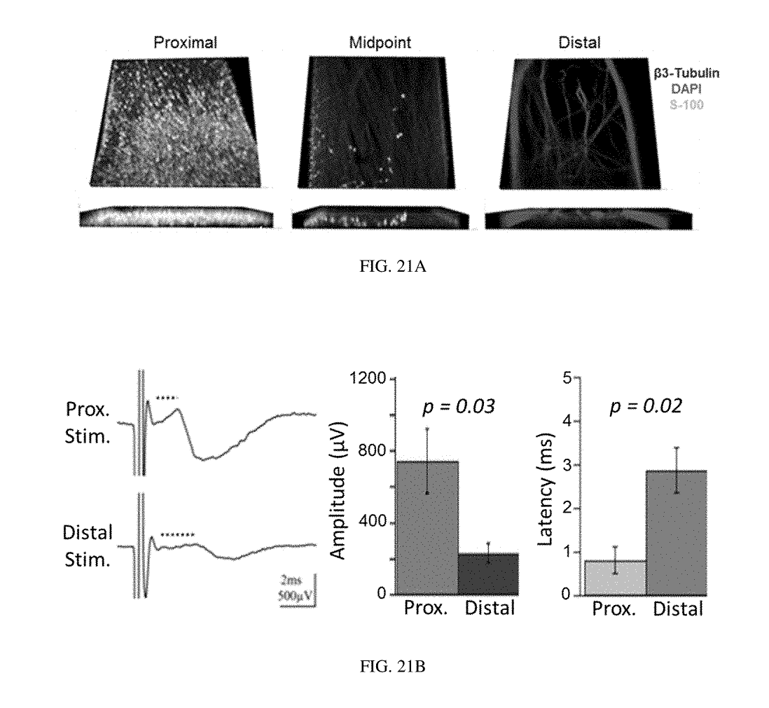

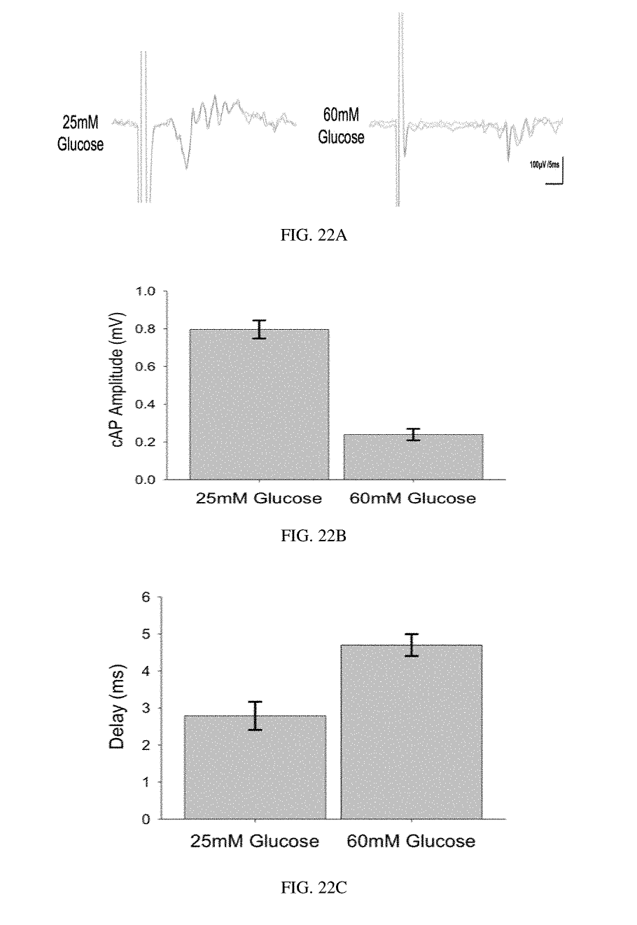

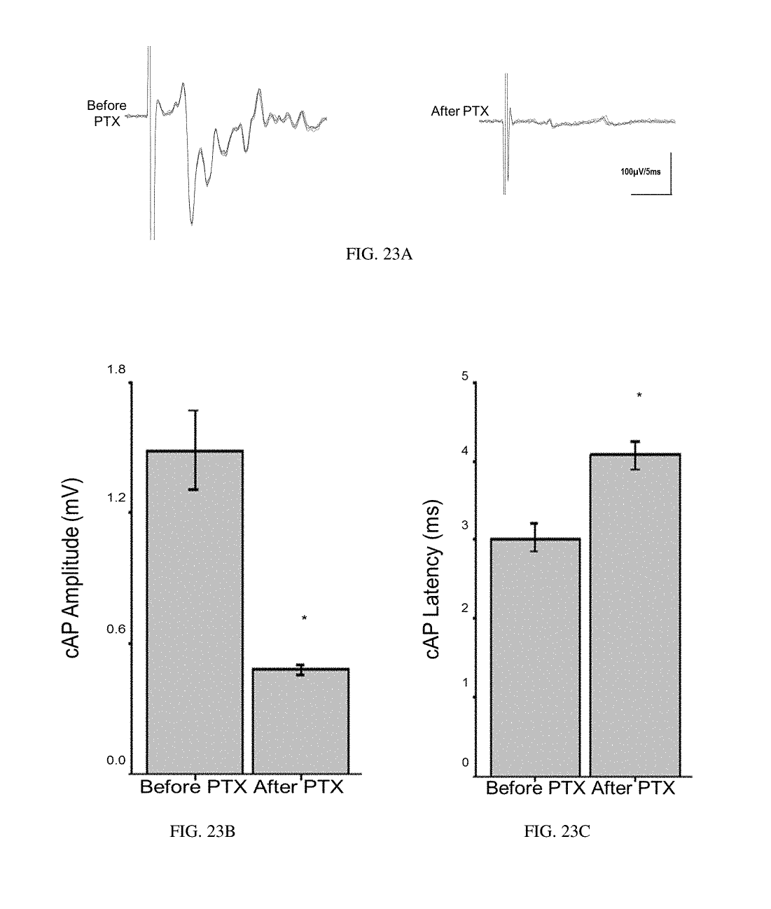

We have developed a simple but unique method of digital projection lithography for rapid micropatterning of one or more hydrogels directly onto conventional cell culture materials (Curley et al 2011, Curley & Moore 2011). Our simple and rapid approach uses two gels: polyethylene glycol (PEG) as a restrictive mold, and crosslinked methacrylated heparin (Me-Hep—we previously used Puramatrix) as a permissive matrix. These dual gels effectively constrain neurite growth from embryonic dorsal root ganglion (DRG) explants within a particular 3D geometry, resulting in axon growth with high density and fasciculation. When cultured in myelin induction medium, we observe a tremendous degree of myelin staining positive for myelin basic protein (MBP), indicating compact myelin, whose characteristic spiral structure is evident from TEM images. The unique structure of this culture model, with a dense, highly-parallel, myelinated, 3D neural fiber tract extending from the ganglion, corresponds to peripheral nerve architecture; it may be assessed using neural morphometry, allowing for clinically-analogous assessment unavailable to traditional cellular assays. Most unique to our nerve-on-a-chip culture models is the ability to record electrically-evoked population field potentials resulting from compound action potentials (cAPs). Traces show characteristic uniform, short-latency population responses, which remain consistent with high frequency (100 Hz) stimulation, show a measurable increase in latency associated with distal tract stimulation (FIGS. 21A and 21B), can be reversibly abolished by tetrodotoxin (TTX), and the responses are insensitive to neurotransmitter blockers, indicating cAPs rather than synaptic potentials (Huval et al 2015). Preliminary evidence indicates that high levels of glucose (60 mM) results in a significantly reduced cAP amplitude along with an increased latency compared to moderate glucose levels (20 mM) (FIGS. 22A-22C). Preliminary evidence also indicates that an acute (48 hr) administration of 0.1 μM Paclitaxel (PTX) results in a significantly reduced cAP amplitude along with an increased latency (FIGS. 23A-23C). This concentration had previously resulted in 50% cell death in conventional DRG cultures, compared to significant measurable cAP changes in our model, suggesting a potentially more informative metric of toxicity. Embryonic DRG cultures have been used effectively as models of peripheral nerve biology for decades (Melli & Hoke 2009). While useful as model systems, conventional DRG cultures are known to be poorly predictive of clinical toxicity when assessed with traditional cell death assays. While single-cell recordings may be obtained from DRGs, we are aware of no reports of recording cAPs, due to the lack of tissue architecture. What makes our model system innovative is the unique ability to assess tissue morphometry and population electrophysiology, analogous to clinical histopathology and nerve conduction testing.

The objective of this project is to demonstrate that certain peripherally neurotoxic chemotherapeutics will induce toxicity in microengineered neural tissue that can be quantified using morphological and physiological measures analogous to clinical metrics. We will approach this objective by first calibrating the model system to determine the baseline variability and characterize structure-function relationships. We will then quantify changes induced by acute application of specific chemotherapeutics known to cause clinical neuropathy in order to demonstrate the technical merit of using the compound action potential (cAP) waveform as a preclinical assay of neurotoxicity.

Aim 1 Rationale and Justification: Traditional assays of neuronal cell viability have not proven useful as pre-clinical screens for neurotoxicity. This is not surprising, since embryonic dorsal root ganglion (DRG) neurons are well known to be far more susceptible to apoptosis than mature nerve cells (Kole et al 2013). Assays of DRG neurite outgrowth may be more relevant as early-stage, high-throughput screens of toxicity (Melli & Hoke 2009). However, a high-content assay useful for differentiating potential neuropathic manifestations and informing lead compound selection remains elusive. Neuronal cells cultured in 3D have been shown to exhibit more biomimetic morphological and electrophysiological behaviors, compared with 2D cultures (Desai et al 2006, Irons et al 2008, Lai et al 2012, Paivalainen et al 2008). Therefore, functional measurements in 3D cultures may be the most promising candidates for such high-content analyses, so long as they are comparable to clinically-relevant organ physiology. Nerve conduction testing has been shown capable of predicting the type and severity of clinical nerve pathology even before symptoms fully manifest (Velasco et al 2014). We propose an analogous electrophysiological metric in the in vitro setting; in order to interpret results, we first need to establish baseline measurements and determine structure-function correlations.

Aim 1 Study Design: Myelinated as well as unmyelinated neural tissue constructs will be fabricated using improvements on our published work (Curley & Moore 2011, Huval et al 2015). Dual hydrogel constructs will be fabricated from PEG gel micromolds filled with Me-Hep gel supplemented with collagen and laminin. Neurite growth constructs will be fabricated to be ˜400 μm wide and up to 5 mm in length. Dorsal root ganglia (DRG) will be taken from thoracic levels of spinal cords dissected from embryonic day 15 (E15) rat embryos and incorporated within bulbar regions of the dual hydrogel constructs. Myelinated tissue constructs will be cultured for 10 days in Basal Eagle's Medium with ITS supplement and 0.2% BSA to promote Schwann cell migration and neurite outgrowth, followed by culture for up to four more weeks in the same medium additionally supplemented with 15% FBS and 50 μg/ml ascorbic acid to induce myelination (Eshed et al 2005). Unmyelinated constructs will be formed by culturing in the same media regimen (outgrowth induction followed by myelin induction), but lacking ascorbic acid. At least two weeks of culture in myelin induction medium, with ascorbic acid, is required for substantial formation of compact myelin. To assess tissue morphology at various stages of maturity, approximately 12 each of myelinated and unmyelinated tissue constructs will be fixed in 4% paraformaldehyde at one, two, three, and four weeks in myelination induction medium (or 17, 24, 31, and 38 total days in vitro, DIV) and stained for nuclei (Hoechst), neurites (βIII-tubulin), Schwann cells (S-100), myelin basic protein (MBP), and apoptosis (Annexin-V and TUNEL). Samples will be imaged with confocal microscopy at regions within the DRG, proximal to the ganglion, near the midpoint of the fiber tract, and in the fiber tract distal to the ganglion; exact distances will be proportional to average maximal neurite extent in each group. After confocal imaging, samples will be post-fixed in 2% osmium tetroxide, dehydrated, and embedded in epoxy resin. Approximately 10 ultrathin cross-sections will be cut from each sample at each defined region (i.e. ganglion, proximal, midpoint, distal) and stained with lead citrate and uranyl acetate for TEM imaging.

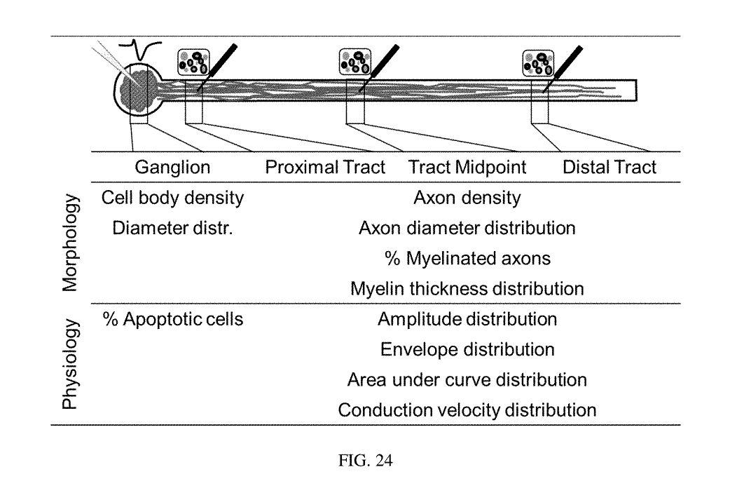

Physiological analysis will be performed as described previously (Huval et al 2015). Both myelinated and unmyelinated constructs will be removed from culture and placed on a field recording rig perfused with artificial cerebral spinal fluid (aCSF). As depicted in FIG. 24, field potential electrodes will be placed in somatic regions of the DRG explants and bipolar stimulating electrodes will be inserted ˜300 μm deep into the channel at distances proximal, near the midpoint, and distal to the ganglion; distances will be informed by morphometry. For each specimen at each stimulation location, stimulation strength will be increased until a characteristic fast (<5 ms), short latency, negative deflecting potential is recorded. DRG spike recordings from each stimulation location will be taken from ˜5-10 specimens at 17, 24, 31, and 38 DIV. These same specimens will be fixed immediately after electrophysiological recording and processed for confocal and TEM analyses.

Morphological analysis will be assessed as summarized in FIG. 24. The density and the diameter distribution of cell bodies will be measured in the ganglion. In the neural fiber tract, measurements will include density and diameter distribution of axons, the % of axons with myelin, and the thickness distribution of myelin sheaths. This analysis will provide important quantitative metrics of morphological variability and for correlation with physiology. The physiological metrics are also summarized in FIG. 24. The cAP will be recorded at three points along the length of the tract, and measurements will include distributions of cAP amplitude (and numbers of peaks), envelope (width), integral (area under the curve), and conduction velocity (from latency). Morphometric parameters of the recorded constructs will be compared against the larger pool of morphometric data to ensure they are within the expected range of variability. We will perform statistical cross-correlation to determine which morphological measures best correlate with which physiological measures (Manoli et al 2014). Additionally, these experiments will provide measures of variability used for a statistical power analysis to determine appropriate sample sizes for Aim 2, and they will be used to define exclusion criteria, e.g. samples with neurite growth more/less than 2 standard deviations from average will be excluded.

Aim 1 Expected Results: We hypothesize that recorded cAP waveforms will reflect morphological observations. For example, our preliminary data suggest that, after two weeks in culture, neurite growth within hydrogel channels was much more dense proximal to the ganglion than distally (FIGS. 21A and 21B). Accordingly, when stimulated proximally vs. distally, the recorded cAPs showed larger amplitude and integral. The latency of the cAP was expectedly longer when stimulating distally, reflecting the conduction time. Conduction velocities calculated were approximately 0.5 m/s, which is unsurprisingly slow, in constructs containing mainly small-diameter, unmyelinated axons.

In the proposed experiments, we expect to see cAP conduction velocities correlating with % myelination and/or axon diameter, while cAP amplitude should correlate with the axon density at the location of stimulation. We will also look for further correlations by observing number of peaks, envelope, and integral, and performing correlation analyses with morphological metrics.

Aim 1 Potential Problems and Alternative Strategies: Preliminary findings strongly demonstrate the technical feasibility of the work proposed in this aim. The most likely anticipated pitfall is that as we measure more cultures, we may find that morphological and/or physiological variability may be too high for many strong correlations to be identified. If this occurs, we will increase sample sizes, as needed, and/or focus our efforts on those metrics representing the strongest correlations. We may also attempt to refine culture conditions to reduce variability, such as by using defined media, or using dissociated cells pooled from multiple animals.

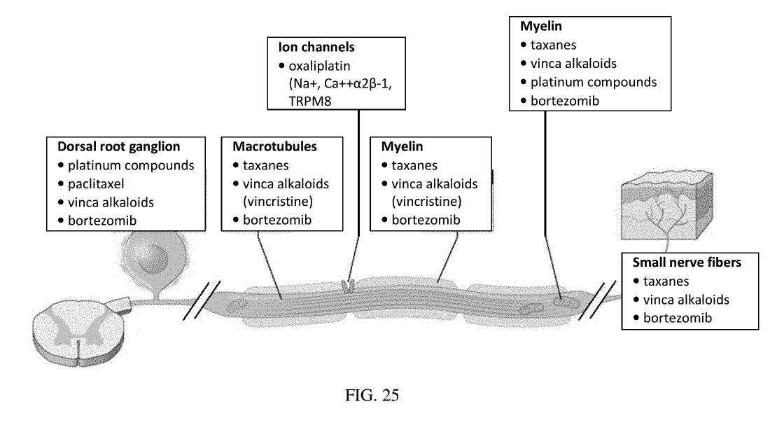

Aim 2 Rationale and Justification: The most commonly administered chemotherapeutics with the most severe documented neurotoxicities are platinum derivatives; tubulin-binding compounds, including vinca alkaloids, taxanes, and epothilones; the proteasome inhibitor bortezomib; and thalidomide (Argyriou et al 2012, Cavaletti & Marmiroli 2010). All of these agents appear to be more toxic to sensory neurons than motor or sympathetic neurons, yet they each target different parts of the nerve, as summarized in FIG. 25, leading to different sets of clinically-measurable histologic and physiologic changes. A high-content, functional assay of toxicity should be able to detect the range of in vivo effects associated with these compounds. To enable a manageable scope, we will restrict experiments to oxaliplatin, vincristine, paclitaxel, and bortezomib. This list ensures an appropriately diverse range of responses, as it includes one compound of each family, excluding epothilones, because they bind tubulins in a manner similar to taxanes, and excluding thalidomide, since it likely involves interactions with other cell types and cytokines (Argyriou et al 2012). We will further restrict experiments to acute application of neurotoxic doses confirmed to be neurotoxic in vitro. Chronic and low-dose administration will be reserved for future detailed studies.

We propose to demonstrate the feasibility of using cAPs as a measure of toxicity by quantifying the morphological and physiological responses to the four chemotherapeutics. The experiments proposed are designed to establish the model with assessments directly analogous to nerve conduction tests as well as clinical histology. Molecular mechanistic studies are beyond the scope of this proposal, but it is important to note that the quasi-3D nature of the micropatterned cultures is amenable to conventional cellular and molecular assays.

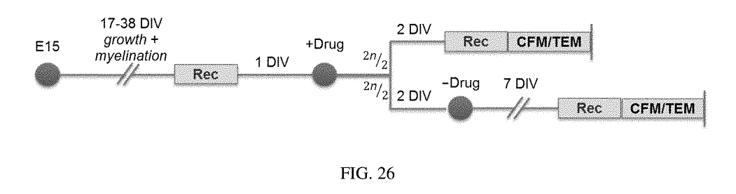

Aim 2 Study Design: We will first perform a small pilot study to ensure the use of effective doses. We will start with doses proven to induce statistically-significant neuronal cell death in vitro after acute application (48-hr) and verify that morphological and physiological changes are measurable in our model at these concentrations. The overall experimental design is summarized in FIG. 26. DRG explants (n=20) will be cultured in micropatterned gels (as described in Aim 1) according to the myelination induction regimen. At a time point determined from Aim 1 to produce fully myelinated constructs, specimens will be checked for sufficient neurite growth (Cell Tracker Green) and myelination (FluoroMyelin Red); specimens without sufficient neurite growth and/or myelination at this point will be excluded from the experiment. Electrophysiological recordings of healthy tissue constructs will be taken, and the next day, neurotoxic concentrations of the four drugs will be applied acutely for 48 hours, as summarized in Table 4. Controls will receive vehicle without drug. Electrophysiology will be performed on half (n=10) of the explants at the end of the 48-hr administration period, and the other half 7 days after the administration period. All specimens will be fixed immediately after the final recording, stained, and assessed as summarized in FIG. 24. Additionally, qualitative observations will be made of soma and axon damage, such as chromatin condensation, blebbing, and axon segmentation.

TABLE 1
Drug doses for initial pilot study.

| Drug | Neurotoxic dose | References |
|---|---|---|
| Oxaliplatin | 15 μM | (Ta et al 2006) |
| Vincristine | 0.1 μM | (Silva et al 2006) |
| Paclitaxel | 0.1 μM | (Scuteri et al 2006) |
| Bortezomib | 0.02 μM | (Luo et al 2011) |

The results of this pilot study will be used to assess the adequacy of dose administration, and doses will be adjusted as needed for the full study (below). The pilot study results will also be used to determine the most strongly correlated morphological and physiological measures, and to perform statistical power analyses to estimate the sample sizes needed to detect ˜10% differences in those measures. In a larger study, we hypothesize that morphological and physiological changes in vitro after acute drug administration will closely parallel in vivo neuropathy as reported in the literature. The objective of this experiment is to catalog a quantifiable neurotoxic signature for each of the drugs in our nerve-on-a-chip model. The full-scale experimental design will mirror the pilot study, as depicted in FIG. 26, but the sample sizes and doses of all four drugs (oxaliplatin, vincristine, paclitaxel, bortezomib) will reflect any changes decided upon from the pilot study.

Aim 2 Expected Results: We hypothesize that acute administration of each drug will induce toxicities that may be detected by measuring changes in cAPs with respect to baseline. We expect most of these changes will correlate with any morphological damage as quantified by our morphometric analysis. For example, referring to FIG. 25, with tubulin-binding drugs vincristine and paclitaxel, we expect to see axonal atrophy, as measured by decreased axon diameter and density, which we expect will accompany decreases in cAP amplitude. We may also see decreases in myelin thickness and % myelinated axons, which may be accompanied by decreases in cAP conduction velocity. With oxaliplatin, we would expect to see higher levels of apoptosis, but less axonal atrophy and myelin damage. Accordingly, while the cAP amplitude may still decrease because of oxaliplatin's effect on Na+ channels, we would not expect to see much of a decrease in conduction velocity without myelin toxicity. We further expect that the physiological and morphological changes will parallel documented clinical pathology as measured by nerve conduction testing and histomorphometry.

Aim 2 Potential Problems and Alternative Strategies: While the neurotoxicity of the four compounds to be tested has already been observed in vitro, the biological effects may be influenced by the 3D preparation in unpredictable ways. It is possible that the kinds of morphological and physiological pathology expected will not manifest in the pilot study, or else cell death will overwhelm functional measures. If so, we may increase/decrease the dose and/or switch to a chronic application (7 days). Another plausible scenario is that the neuropathy will be evident but quantitative measures so variable as to make 10% detectable differences impractical. If so, we will design the larger study to detect a 20%-30% detectable difference, as is practical.

### REFERENCES

Argyriou A A, Bruna J, Marmiroli P, Cavaletti G. 2012. Chemotherapy-induced peripheral neurotoxicity (CIPN): an update. Critical reviews in oncology/hematology 82: 51-77. doi: 10.1016/j.critrevonc.2011.04.012. PubMed PMID: 21908200.
Astashkina A, Grainger D W. 2014. Critical analysis of 3-D organoid in vitro cell culture models for high-throughput drug candidate toxicity assessments. Advanced drug delivery reviews 69-70: 1-18. doi: 10.1016/j.addr.2014.02.008. PubMed PMID: 24613390.
Cavaletti G, Marmiroli P. 2010. Chemotherapy-induced peripheral neurotoxicity. Nature reviews. Neurology 6: 657-66. doi: 10.1038/nrneuro1.2010.160. PubMed PMID: 21060341.
Curley J L, Jennings S R, Moore M J. 2011. Fabrication of micropatterned hydrogels for neural culture systems using dynamic mask projection photolithography. Journal of visualized experiments 48: e2636. doi: 10.3791/2636. PubMed PMID: 21372777; PMCID: 3197419.
Curley J L, Moore M J. 2011. Facile micropatterning of dual hydrogel systems for 3D models of neurite outgrowth. Journal of biomedical materials research. Part A 99: 532-43. doi: 10.1002/jbm.a.33195. PubMed PMID: 21936043; PMCID: 3213030.
Desai A, Kisaalita W S, Keith C, Wu Z Z. 2006. Human neuroblastoma (SH-SY5Y) cell culture and differentiation in 3-D collagen hydrogels for cell-based biosensing. Biosensors & bioelectronics 21: 1483-92. doi: 10.1016/j.bios.2005.07.005. PubMed PMID: 16084714.
DiMasi J A, Grabowski H G, Hansen R W. 2014. Cost to develop and win marketing approval for a new drug is $2.6 billion: Tufts Center for the Study of Drug Development, http://csdd.tufts.edu/.
Dyck P J, Thomas P K. 2005. Peripheral Neuropathy. Philadelphia: Saunders.
Emami J. 2006. In vitro—in vivo correlation: from theory to applications. Journal of pharmacy & pharmaceutical sciences 9: 169-89. doi. PubMed PMID: 16959187.
Eshed Y, Feinberg K, Poliak S, Sabanay H, Sarig-Nadir 0, et al. 2005. Gliomedin mediates Schwann cell-axon interaction and the molecular assembly of the nodes of Ranvier. Neuron 47: 215-29. doi: 10.1016/j.neuron.2005.06.026. PubMed PMID: 16039564.
Ghaemmaghami A M, Hancock M J, Harrington H, Kaji H, Khademhosseini A. 2012. Biomimetic tissues on a chip for drug discovery. Drug discovery today 17: 173-81. doi: 10.1016/j.drudis.2011.10.029. PubMed PMID: 22094245; PMCID: 3273552.
Huval R M, Miller O H, Curley J L, Fan Y, Hall B J, Moore M J. 2015. Microengineered peripheral nerve-on-a-chip for preclinical physiological testing. Lab On A Chip In press, 10.1039/C4LC01513D.
Irons H R, Cullen D K, Shapiro N P, Lambert N A, Lee R H, Laplaca M C. 2008. Three-dimensional neural constructs: a novel platform for neurophysiological investigation. Journal of neural engineering 5: 333-41. doi: 10.1088/1741-2560/5/3/006. PubMed PMID: 18756031.
Kimlin L, Kassis J, Virador V. 2013. 3D in vitro tissue models and their potential for drug screening. Expert opinion on drug discovery 8: 1455-66. doi: 10.1517/17460441.2013.852181. PubMed PMID: 24144315.
Kola I, Landis J. 2004. Can the pharmaceutical industry reduce attrition rates? Nature reviews. Drug discovery 3: 711-5. doi: 10.1038/nrd1470. PubMed PMID: 15286737.
Kole A J, Annis R P, Deshmukh M. 2013. Mature neurons: equipped for survival. Cell death & disease 4: e689. doi: 10.1038/cddis.2013.220. PubMed PMID: 23807218; PMCID: 3702294.
Lai Y, Cheng K, Kisaalita W. 2012. Three dimensional neuronal cell cultures more accurately model voltage gated calcium channel functionality in freshly dissected nerve tissue. PloS One 7: e45074. doi: 10.1371/journal.pone.0045074. PubMed PMID: 23049767; PMCID: 3458113.
Li A P. 2004. Accurate prediction of human drug toxicity: a major challenge in drug development. Chemico-biological interactions 150: 3-7. doi: 10.1016/j.cbi.2004.09.008. PubMed PMID: 15522257.
Luo P, Lin M, Li L, Yang B, He Q. 2011. The proteasome inhibitor bortezomib enhances ATRA-induced differentiation of neuroblastoma cells via the JNK mitogen-activated protein kinase pathway. PloS One 6: e27298. doi: 10.1371/journal.pone.0027298. PubMed PMID: 22087283; PMCID: 3210155.
Malik B, Stillman M. 2008. Chemotherapy-induced peripheral neuropathy. Current neurology and neuroscience reports 8: 56-65. doi. PubMed PMID: 18367040.
Manoli T, Werdin F, Gruessinger H, Sinis N, Schiefer J L, et al. 2014. Correlation analysis of histomorphometry and motor neurography in the median nerve rat model. Eplasty 14: e17. doi. PubMed PMID: 24904711; PMCID: 3984537.
Melli G, Hoke A. 2009. Dorsal Root Ganglia Sensory Neuronal Cultures: a tool for drug discovery for peripheral neuropathies. Expert opinion on drug discovery 4: 1035-45. doi: 10.1517/17460440903266829. PubMed PMID: 20657751; PMCID: 2908326.
Moya del Pino B. 2010. Chemotherapy-induced Peripheral Neuropathy. NCI Cancer Bulletin:
Paivalainen S, Nissinen M, Honkanen H, Lahti O, Kangas S M, et al. 2008. Myelination in mouse dorsal root ganglion/Schwann cell cocultures. Molecular and cellular neurosciences 37: 568-78. doi: 10.1016/j.mcn.2007.12.005. PubMed PMID: 18206387.
Schuster D, Laggner C, Langer T. 2005. Why drugs fail—a study on side effects in new chemical entities. Current pharmaceutical design 11: 3545-59. doi. PubMed PMID: 16248807.
Scuteri A, Nicolini G, Miloso M, Bossi M, Cavaletti G, et al. 2006. Paclitaxel toxicity in post-mitotic dorsal root ganglion (DRG) cells. Anticancer research 26: 1065-70. doi. PubMed PMID: 16619507.
Silva A, Wang Q, Wang M, Ravula S K, Glass J D. 2006. Evidence for direct axonal toxicity in vincristine neuropathy. Journal of the peripheral nervous system 11: 211-6. doi: 10.1111/j.1529-8027.2006.0090.x. PubMed PMID: 16930282.
Ta L E, Espeset L, Podratz J, Windebank A J. 2006. Neurotoxicity of oxaliplatin and cisplatin for dorsal root ganglion neurons correlates with platinum-DNA binding. Neurotoxicology 27: 992-1002. doi: 10.1016/j.neuro.2006.04.010. PubMed PMID: 16797073.
Velasco R, Bruna J, Briani C, Argyriou A A, Cavaletti G, et al. 2014. Early predictors of oxaliplatin-induced cumulative neuropathy in colorectal cancer patients. Journal of neurology, neurosurgery, and psychiatry 85: 392-8. doi: 10.1136/jnnp-2013-305334. PubMed PMID: 23813745.
Wang X M, Lehky T J, Brell J M, Dorsey S G. 2012. Discovering cytokines as targets for chemotherapy-induced painful peripheral neuropathy. Cytokine 59: 3-9. doi: 10.1016/j.cyto.2012.03.027. PubMed PMID: 22537849.
Windebank A J, Grisold W. 2008. Chemotherapy-induced neuropathy. Journal of the peripheral nervous system 13: 27-46. doi: 10.1111/j.1529-8027.2008.00156.x. PubMed PMID: 18346229.

### Example 4: Retinal Explant Model (Non-Prophetic)

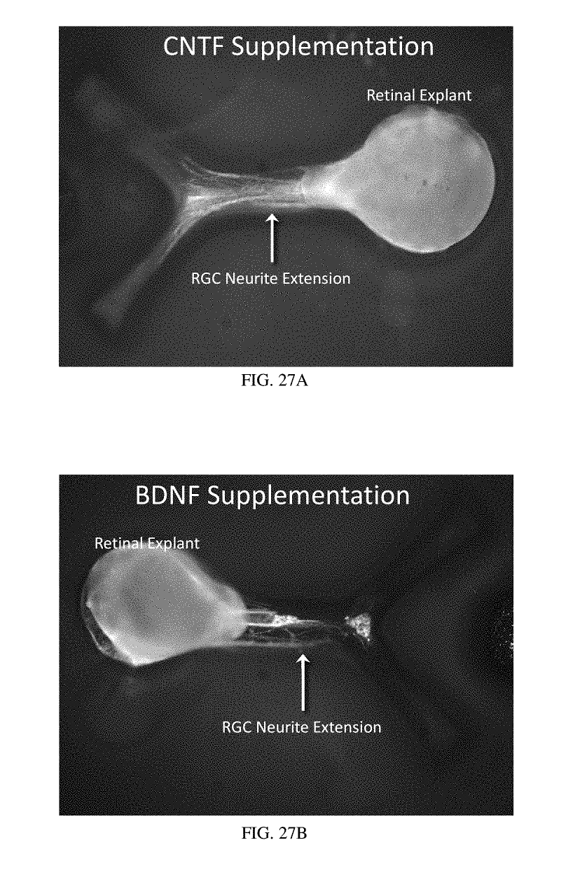

Work and experimental data for the dorsal root ganglia model is presented in the present disclosure. For a central nervous system model, the growth of retinal explants has also been explored. FIGS. 27A-27B depict a culture of retinal (CNS) tissue. Retinal explants from embryonic rats were cultured within 3D micropatterned hydrogels in “neurobasal Sato” medium supplemented with either ciliary neurotrophic factor CNTF (FIG. 23A) or brain-derived neurotrophic factor BDNF (FIG. 23B). Observable retinal ganglion cell axon extension was visualized after one week in culture, stained with β-III tubulin.

### Example 5: Thalamio-Cortical Model (Prophetic)

One embodiment of the present invention quantifies evoked postsynaptic potentials in a biomimetic, engineered thalamocortical circuit. DLP lithography is used to cure micromolds of 10% polyethylene glycol diacrylate (PEG) gels approximately 500 μm thick. The molds contain two reservoirs ˜500 μm in diameter separated by a tract ˜200 μm wide and ˜1 mm long. Thalamic and cortical neurons are isolated from E18 rat embryos, dissociated with trypsin/papain, triturated, and pelleted using common procedures. A concentrated cell suspension (˜5E6 cells/ml) in Puramatrix gel is formed by resuspending pellets in a 10% sucrose solution and combining with an equal volume of 0.3% Puramatrix and 10% sucrose. Respective thalamic and cortical cell suspensions are placed in individual reservoirs within each mold via micropipette, and Puramatrix with no cells is placed in the space between. The micropatterned co-culture constructs are cultured for up to two weeks and circuits allowed to form spontaneously. At intervals of ˜3 days, constructs are fixed and stained for cell nuclei (DAPI), neurites (β3-tubulin), dendrites (MAP2) and synapses (synapsin) in order to determine the time course necessary for production of a circuit. Subsequently, pastes of the lipophilic tracing dyes Di-I and Di-O are placed in either end of the constructs, which are fixed before synapse formation takes place, in order to determine the prevalence and organization of neurite growth from either cell population. These morphological parameters are quantified with confocal analysis and used to finalize the design of the microengineered circuit. This produces a high and reproducibly uniform density of thalamic axons synapsing onto a defined population of post-synaptic cortical neurons, while minimizing corticothalamic re-innervation (<10% of synapses).

Next, the electrophysiological characteristics of the circuits are determined. A single bipolar stimulating electrode is used to activate both antidromically propagating action potentials (APs) and orthodromically evoked excitatory synaptic potentials (EPSPs) in these TC circuits. Responses are measured by both field potential and whole-cell voltage-clamp recording. Antidromic action potentials are recorded to confirm the induction and propagation of active currents in these axons. Consistent with results from our DRG constructs we expect to be able to record antidromic APs using field potential electrodes in the thalamic neuron pool. This is seen as short and consistent latency, TTX-sensitive, negative deflecting, field potentials of short duration. Whole-cell voltage recordings are used to verify these antidromic APs based upon their kinetics, direct onset from baseline, and insensitivity to hyperpolarization. Glutamatergic EPSPs and excitatory post-synaptic currents (EPSCs) in the cortical neuron population are then be confirmed following bipolar stimulation of the thalamic axons. EPSCs are confirmed using 1) kinetic analysis of field potential responses recorded in the cortical neuron pool, 2) whole-cell current recordings employing voltage-clamping strategies to isolate AMPAR-mediated (at hyperpolarized holding potentials) and AMPAR+NMDAR-mediated currents (at positive holding potentials), and 3) standard glutamatergic synapse pharmacology including DNQX (20 μM) to selectively block AMPARmediated currents and d-APV (50 μM) to antagonize NMDAR-mediated currents. AMPAR- and NMDAR mediated post-synaptic currents in response to thalamic axon stimulation then occur. The relative ratio of AMPAR- to NMDAR-mediated current will increase over these two weeks in vitro mimicking the in vivo situation.

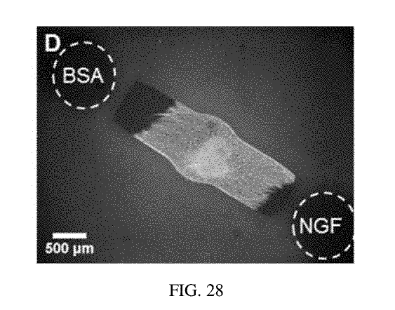

In some embodiments, the trophic actions of cortical neurons on thalamic cells are not sufficient for formation of the desired unidirectional circuit. In these embodiments, corticothalamic reinnervation is consistently above 10%, or dendritic arbors connect between the two cell populations. If these scenarios are observed to an undesirable degree, the timing of the introduction of each cell type are staggered such that the thalamic neurons are introduced and given time to generate and extend axons toward the cortical neuron reservoir before addition of the cortical target neurons. Alternatively, or in conjunction, the micropatterning ability of the hydrogel is used to introduce artificial trophic signaling during culture. We have shown that DRG neurites grow preferentially toward NGF, as opposed to BSA, diffusing from a reservoir in the hydrogel construct, as shown in FIG. 28. Potential chemo-attractant molecules for TC axons include netrin-1 and neurotrophin3. In a similar fashion, semaphorin 3A is used, since it has been shown to polarize cortical neurons by attracting dendrites and repelling axons. If these approaches are not effective, a photodegradable version of the PEG hydrogel is used, which we have been able to synthesize. This gel allows placement of a PEG barrier between cortical and thalamic pools, which can be degraded with UV light to allow synapse formation when desired.

### Example 6: Combination of Microphysiological Culture System and Non-Invasive Electrophysiological Analysis (Prophetic)

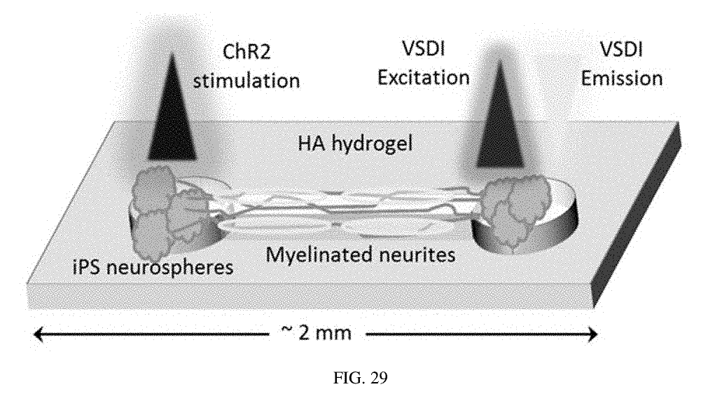

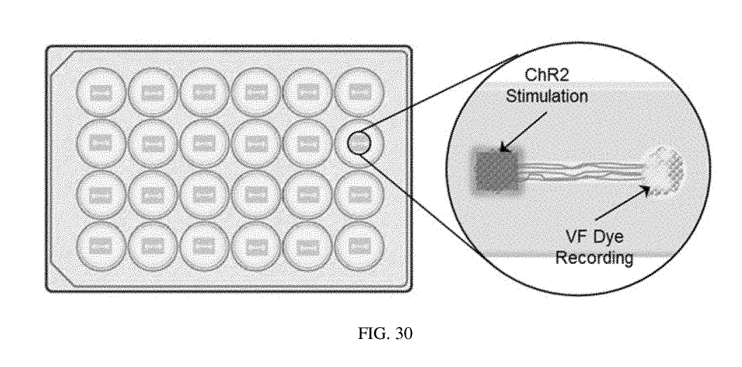

One embodiment of the present invention is to utilize the unique combination of microphysiological culture systems and noninvasive electro-physiological analyses. This has potentially paradigm-changing ability to perform population-level, functional assays in biomimetic configurations in vitro. We have manually configured a DLP device on a fluorescence microscope recording rig and have shown selective illumination and simultaneous activation of individual cortical neurons as well as individual dendrites in cells expressing GFP and ChR2. We have also developed custom software for flexible user control of illumination by enabling the designation of regions of interest directly on the microscope camera's live feed, as seen by the user. This powerful and versatile application of DLP microscopy and optogenetics for optical neuroactivation is combined it with a new form of voltage-sensitive dye imaging, such as VF. This unique and timely combination of optogenetics and VF imaging with DLP microscopy represents a powerful, completely-optical method for noninvasive stimulation of our microengineered circuits; FIG. 29

In one embodiment, DLP optical stimulation and recording protocols is worked out in traditional, planar dissociated cultures of thalamic and cortical neurons, respectively. Cortical and thalamic cultures are generated using methods described above. We will use ChR2 plasmid- and lentiviral-based DNA constructs, which we have obtained from Optogenetics, Inc., and that include a red fluorescent protein (mCherry) as a transfection/infection reporter. Neurons are plated and infected with ChR2 and then stained with VF dye (2 μM). Whole-cell patch recordings are then established on a transfected/infected cell, and then DLP illuminated at ˜475 nm (blue-green). Graded potentials and action potentials will be recorded in current clamp mode while varying illumination intensity and magnification (4×-40×). Alternatively, voltage is clamped to variable potentials while VF fluorescence is monitored at ˜535 nm (yellow-green; VF is relatively insensitive to excitation wavelength), again while also varying excitation intensity and magnification. These tests are repeated to determine the ranges and limits of illumination and voltage sensitivities. Additionally, in this example the timing requirements for simultaneous illumination (or nearly simultaneous) for optical stimulation and recording are determined. For evoking and recording synaptic potentials, low-density cortical cultures are generated. This manipulation (approximately 10-100 k cells/mL) is required to maximize connectivity and get connected neurons in individual fields of view in these cortical cultures. After establishing a whole cell patch, transfected/infected neighboring cells are then illuminated with DLP and postsynaptic potentials will be recorded in current-clamp mode. These experiments are used to determine the precise optical setup, illumination, and timing of optical sampling required to detect ChR2/light-evoked postsynaptic potentials.

Optical stimulation and recording protocols are next worked out in 3D cell populations. Stimulation and recording are at relatively low magnification (10×) so that the thalamic and cortical pools are at once visible within the field of view. TC circuits are microengineered according to methods above. However, thalamic cells are infected with ChR2 virus by adding particles to the cell suspension in Puramatrix solution before injection into PEG micromolds, and then gels washed several times to remove particles before addition of cortical neurons. Stimulating field electrodes are placed in thalamic neuron pools, and recording electrodes in cortical neuron pools, and the ability to evoke EPSPs is confirmed. Immediately following, DLP illumination of ChR2 is used to stimulate thalamic neurons while recording responses in the cortical pool. EPSP responses to varying presynaptic ChR2 illumination intensities at different magnifications (4×-40×) is investigated. In some embodiments, EPSPs are confirmed with field recordings in TC circuits with VF-stained cortical neurons, and immediately following, electrically-evoked postsynaptic responses in cortical pools are measured by VF fluorescence upon stimulation of thalamic neurons with field electrodes. Fluorescence measurements of EPSPs are characterized by kinetic analysis and glutamatergic synapse pharmacology. Finally, shortly after confirmation with field stimulation and recording, thalamic neurons are stimulated with ChR2 while cortical EPSPs are measured with VF.

Depending on the techniques determined for circuit fabrication, viral ChR2 infection in the hydrogels may pose a problem, either because of reduced infection efficiency or residual virus in the gel causing undesired infection of cortical neurons. Viral infection is preferred because it is expected to yield the highest efficiency, but, in other embodiments, chemical transfection and electroporation methods may be used as well. If necessary, thalamic cells may be plated conventionally for infection, washed thoroughly, then dissociated and suspended in Puramatrix. If it is not be possible to balance low magnification, required for visualization of the entire TC circuit, with SNR, required for resolving VF fluorescence at high speeds, alternative equipment configurations, including specialized objectives with low magnification and high numerical apertures, and cameras (CCDs or PMTs) with higher speed and sensitivity are used. Alternatively, in other embodiments, fiber optic application of light for ChR2 stimulation independent of the microscope light path is used.

### Example 7: High-Throughput Format for Culture System (Prophetic)

One embodiment of the invention would be for a multiwell format as depicted in FIG. 30. In one embodiment, a fluorescence microscope and electrophysiology rig will be configured. An epifluorescent microscope and recording platform is configured, comprising a fixed-stage, upright microscope with digital interference contrast (“DIC”) and fluorescence optics, and coarse and fine micromanipulators for placement of stimulation electrodes and recording electrodes, respectively. Field potential and whole-cell amplifiers are complemented with digital stimulation capabilities to allow electrode-based microelectrode analysis, for required confirmation of optical activation and recording. Additionally, the microscope is equipped with a DLP adaptive illuminator (Andor Technology, plc.), fast solid-state multispectral light sources (such as the SPECTRA X Light Engine™ by Lumencor, Inc.), and an interface for synchronization of DLP, light sources, and camera. Control of the system will be achieved through a combination of commercial software in communication with a custom LabView interface for illumination and imaging, and IgorPro for data acquisition and analysis.

Microengineered DRG constructs may be fabricated as described above, and grown and recorded in a standard six-well tissue culture plate format. The size of these current constructs is highly amenable to fast screening. In one embodiment, it is preferred to stabilize signal consistency by maximizing the density of cultured tissue. By generating simple monosynaptic circuits it is possible to increase the target cell pool to offset this issue. In terms of illumination, for stimulation and recording, the strength of the DLP system is its adaptability. Software will create the ability to spatially pattern the illumination and recording within the field of view.

In one embodiment, the constructs are fabricated in 24 well plate formats. In other embodiments, 96 well plates are used. At each stage response amplitudes and consistency of responses are examined, as well as individual variability between wells under control conditions. A balance is determined between the speed of analysis and the number of constructs that need to be recorded to minimize variability enough to see a biologically relevant change in synaptic transmission. To do this controlled modifications are made in test wells to examine determined changes in transmission. For example, 100% suppression of transmission by addition of 20 μM DNQX+50 μM APV in these constructs will provide a negative control. More fine scale manipulations are also be performed, for example addition of cyclothiazide to remove basal levels of AMPAR desensitization can be used to enhance transmission at these synapses by approximately 10-20%. For each manipulation the average degree of suppression or enhancement of transmission is confirmed using electrode-based electrophysiology. The number of constructs we need to measure optically is determined in order to reliably record this % change in transmission for each condition. Following functional assessments, the TC circuits are fixed and a random sample chosen for morphological assessment. Constructs are stained for cell nuclei, neurites, dendrites, and synapses. The relative densities of these morphological parameters are quantified with confocal microscopy, and correlations between morphological and functional variability are investigated, which aid the refinement of fabrication procedures. The main advantage of this assay is the advancement in recording by removing the requirement for micro-electrode placement to record biologically relevant synaptic potentials.

In some embodiments, where fabrication proves to be the limiting factor, cell printing with ink-jet style deposition of cells, perhaps in combination with projection lithography is used. If fluid handling proves to be a bottleneck, robotic pipetting systems or other automated fluid handlers is employed.

### Example 8: Effects of Therapeutics on Neurotransmission (Prophetic)

In one embodiment, the invention is used to test the effects of therapeutics on neurotransmission. In one embodiment, for both chronic and acute exposure, TC constructs are prepared. For chronic experiments, constructs are grown until the initial point of TC axonal innervation of the cortical neurons at which point experimental cultures are treated with an exogenous source of 5-HT either alone or in conjunction with one of the pharmaceuticals from our panel (FIG. 16). The time point associated with innervation of the cortical neurons is determined in examples 1 and 2. As a control, cultures are also included that are not supplied with an exogenous supply of 5-HT. Comparison between 5-HT lacking and 5-HT only cultures are used to demonstrate the requirement of this serotonergic signaling in the development of synaptic transmission at these synapses. Any observed effect of 5-HT is confirmed by reversing these changes with co-application of 5-HT receptor antagonists. Cultured constructs are generated and maintained simultaneously under identical conditions, to minimize experimental variability.

The effect of 5-HT on the development of normal synaptic function is examined by comparing 5-HT and 5-HT lacking (media only) cultures. The duration of chronic treatment for the experimental drugs is determined based upon the time course and strength of 5-HT-mediated changes on synaptic responses. The following synaptic response parameters are measured in the recording phase using VSD stained cortical neurons and channel-rhodopsin-mediated stimulation of thalamic axons: 1) the level of spontaneous excitatory post-synaptic potentials both in terms of their frequency and amplitude of events, 2) the amplitude and kinetics as well as the stimulus response relationship for channel-rhodopsin evoked postsynaptic potentials, and 3) the pharmacology of excitatory synaptic potentials. These pharmacology measurements are used to verify the proper progression of AMPAR- to NMDAR-mediated synaptic current at these synapses, which increases over development. Multiple constructs per condition are recorded to allow statistical measurement. 5-HT enhances the development of synaptic properties including spontaneous activity and an increase in AMPAR/NMDAR current ratio. Treatments that are known to enhance spontaneous activity as a positive control are used to confirm our ability to record these changes using our optical methods. For example, 3 days of TTX treatment which is known to scale up both the amplitude and frequency of spontaneous synaptic responses in cortical cultured neurons.

The present invention tests if 5-HT will be required for the normal development of synaptic transmission at these synapses. However, if there is no effect of chronically blocking SERT this would suggest an interesting dissociation between acute neurotransmission and the developmental spatial patterning of these synaptic inputs. Fluoxetine concentrations will initially be tested at 1,3 and 5 μg/mL as per previous studies. For each condition, data is gathered using optical activation and recording techniques developed in the previous examples, drugs are applied as per previous literature, and the same three parameters are measured.

Potential variation in response parameters due to changes in axon guidance (and therefore strength of cortical innervation by the thalamic neurons), is minimized by applying drugs after initial innervation (7-14 DIV) and by recording multiple constructs per experimental condition. Axonal outgrowth is examined by immunostaining cultures for the axonal protein marker, tau, and quantitatively measuring the intensity of staining in cortical neurons in each treatment condition. Synaptic staining in post-hoc experiments allow us to compare synapse number with these manipulations and allow us to interpret the voltage sensitive dye recordings in terms of increased synapse number and increased strength of individual synapses. Synapses are determined by examining co-localization of presynaptic markers (Vglut ½ mixed antibody) and PSD-95 stain to identify postsynaptic structures. In addition, data is confirmed in initial studies by electrical recordings and immunohistochemistry as appropriate.

If serotonin rapidly modifies synaptic function at these synapses bidirectional, opposing changes in baseline glutamatergic transmission should be observed in response to application of SSRIs or the 5-HT antagonists. Interestingly, there is evidence that SSRIs have rapid effects on synaptic transmission that are independent of their effect on serotonin reuptake. These effects would be expected to occur during much faster time scales. For example, fluoxetine can inhibit T-type, N-type and L-type Ca2+ currents, Na+ current, and K+ currents. For this reason, the acute effects of all these drugs on excitatory synaptic transmission are examined. In these acute experiments, baseline recordings are made for 10 min and then drugs are added for 10 min followed by a 10 min wash out period. Stimuli are evoked and recorded at 0.1 Hz throughout. For these acute recordings the amplitude and kinetics of post-synaptic responses are measured to determine the potential effect of these drugs on synaptic transmission.

The use of purely optical stimulation and recording in this assay allows the rapid screening of the effects of both acute and chronic exposure of these drugs and allows testing of both the absolute sensitivity and dose dependence effects of these drugs on excitatory synaptic function. Compiled data is analyzed by automated routines and the results provide a foundation for understanding both the acute and chronic effects of serotonin modulation on glutamatergic synapse function at developing TC synapses. In some embodiments, by measuring the modulation potential of these drugs in our synapse assays and comparing with prevalence of side effects in vivo, this assay is used to screen novel molecules and peptides with regards to their ability to modify serotonergic function while minimizing ‘off target’ effects such as altering glutamatergic synaptic function.

In addition to large volume, high-throughput screening, in some embodiments, this system can also be used for mechanistic work by rapidly examining the effect of small molecules and known pharmaceutical agents on an observed effect. For example, the requirement of different downstream signaling pathways in regulating synaptic function by SSRIs can be determined by co-applying compounds that block specific cellular pathways or receptor subtypes. In addition to voltage recordings, calcium loading of pre- or post-synaptic neurons can be applied to look at terminal calcium changes and compare this with functional changes in transmitter release. Furthermore, in some embodiments, application of alternate stimulation paradigms can easily be applied to test for changes in parameters such as presynaptic release probability, by measuring paired pulse ratios, and applying tetanizing stimuli to evoke potentiation and screen for modulators of the plasticity. In some embodiments, the use of automated media systems such as automated pipetting machines and/or built-in fluid chambers for cell incubators, allows for the removal of manual manipulation of drug applications and media removal.

1. A method of producing a three-dimensional culture of one or a plurality of neuronal cells in a culture vessel comprising a solid substrate, said method comprising:
(a) contacting one or a plurality of isolated Schwann cells and/or oligodendrocytes with the solid substrate, said substrate comprising at least one exterior surface, at least one interior surface and at least one interior chamber defined by the at least one interior surface and accessible from a point exterior to the solid substrate through at least one opening;
(b) seeding one or a plurality of isolated neuronal cells or tissue explants comprising neuronal cells to the at least one interior chamber;
(c) applying a cell medium into the culture vessel with a volume of cell medium sufficient to cover the at least one interior chamber;
wherein at least one portion of the interior surface comprises a first cell-impenetrable polymer and a first cell-penetrable polymer.

2. The method of claim 1, wherein step (a) is preceded by placing a solution comprising the first cell-impenetrable polymer and the first cell-penetrable polymer into the culture vessel and inducing the first cell-impenetrable polymer and the first cell-penetrable polymer to physically adhere or chemically bond onto at least a portion of the interior surface.

3. (canceled)

4. The method of claim 1, wherein the base comprises one or a combination of silica, plastic, ceramic, or metal and wherein the base is in a shape of a cylinder or in a shape substantially similar to a cylinder, such that the first cell-impenetrable polymer and a first cell-penetrable polymer coat the interior surface of the base and define a cylindrical or substantially cylindrical interior chamber or compartment; and wherein the opening is positioned at one end of the cylinder.

5.-6. (canceled)

7. The method of claim 1, wherein the first cell-impenetrable polymer is polyethylene glycol (PEG) at a concentration of no more than about 20% weight to volume of the solution.

8. The method of claim 1, wherein the first cell-penetrable polymer is at a concentration of from about 0.1% to about 3.0% in weight in volume of the solution.

9.-10. (canceled)

11. The method of claim 1, wherein step (c) comprises seeding tissue explants selected from one or a combination of: an isolated dorsal root ganglion, a spinal cord explant, a retinal explant, and a cortex explant.

12. The method of claim 1, step (c) comprises seeding a suspension of neuronal cells selected from one or a combination of: motor neurons, cortical neurons, spinal cord neurons, peripheral neurons.

13. The method of claim 1, wherein the solid substrate comprises a plastic base cross-linked with a mixture of the first cell-impenetrable polymer and the first cell-penetrable polymer; and wherein the plastic base comprises a plurality of pores with a diameter of no greater than about 1 micron.

14.-19. (canceled)

20. The method of claim 1, wherein the solid substrate comprises no greater than about 15% PEG and from about 0.05% to about 1.00% of one or a combination of self-assembling peptides chosen from: RAD 16-I, RAD 16-11, EAK 16-I, EAK 16-II, and of dEAK 16.

21. (canceled)

22. The method of claim 1, wherein the solid substrate polymer is free of PEG.

23.-24. (canceled)

25. The method of claim 1 further comprising positioning at least one stimulating electrode at or proximate to soma of the one or plurality of neuronal cells or tissue explants and positioning at least one recording electrode at or proximate to an axon at a point most distal from the soma, such that. upon introducing a current in the stimulating electrode, the recording electrode is capable of receiving a signal corresponding to one or a plurality of electrophysiological metrics capable of being measured at the recording electrode.

26. (canceled)

27. A composition comprising:
(i)
a culture vessel;
a hydrogel matrix comprising at least a first cell-impenetrable polymer and a first cell-penetrable polymer; and
one or a plurality of isolated Schwann cells and/or one or a plurality of oligodendrocytes; and
one or a plurality of tissue explants or fragments thereof; or
(ii)
a culture vessel;
a hydrogel matrix comprising at least a first cell-impenetrable polymer and a first cell-penetrable polymer; and
one or a plurality of isolated Schwann cells and/or one or a plurality of oligodendrocytes; and
a suspension of cells comprising one or a plurality of neuronal cells.

28. The composition of claim 27 further comprising a solid substrate onto which the hydrogel matrix is crosslinked, said solid substrate comprising at least one predominantly plastic surface with pores from about 1 micron to about 5 microns in diameter.

29.-31. (canceled)

32. The composition of claim 27 further comprising a solid substrate with a contiguous exterior surface and an interior surface, such solid substrate comprising at least one portion in a cylindrical or substantially cylindrical shape and at least one hollow interior defined at its edge by at least one portion of the interior surface, said interior surface comprising one or a plurality of pores from about 0.1 microns to about 1.0 microns in diameter wherein the hollow interior of the solid substrate is accessible from a point exterior to the solid substrate through at least one opening; wherein the hollow interior portion comprises a first portion proximate to the opening and at least a second portion distal to the opening; wherein the one or plurality of neuronal cells and/or the one or plurality of tissue explants are positioned at or proximate to the first portion of the hollow interior and are in physical contact with the hydrogel matrix, and wherein the second portion of the at least one hollow interior is in fluid communication with the first portion such that axons are capable of growth from the one or plurality of neuronal cells and/or the one or plurality of tissue explants into the second interior portion of the hollow interior.

33. The composition of claim 27, wherein the composition is free of a sponge or PEG.

34. The composition of claim 27, wherein the at least one cell-impenetrable polymer comprises no greater than about 15% PEG and the at least one cell-penetrable polymer comprises from about 0.05% to about 1.00% of one or a combination of self-assembling peptides chosen from: RAD 16-I, RAD 16-II, EAK 16-I, EAK 16-II, and dEAK 16.

35. The composition of claim 27, wherein the culture vessel comprises 96, 192, 384 or more interior chambers in which one or plurality of isolated Schwann cells and/or one or plurality of oligodendrocytes are sufficiently proximate to the one or plurality of isolated tissue explants and/or the one or plurality of neuronal cells such that the Schwann cells or the oligodendrocytes deposit myelin to axon growth from the tissue explants and/or neuronal cells.

36.-44. (canceled)

45. The composition of claim 27, wherein the one or plurality of tissue explants comprises one or a plurality of DRGs with axonal growth from about 100 microns to about 500 microns in width and from about 0.11 to about 10000 microns in length.

46. (canceled)

47. A method of assessing a response from one or more neuronal cells comprising:
growing one or more neuronal cells in a culture vessel;
introducing one or more stimuli to the one or more neuronal cells; and
measuring one or more responses from the one or more neuronal cells to the one or more stimuli.

48.-55. (canceled)

56. The method of claim 47, wherein the one or more neuronal cells comprise isolated primary ganglion tissue.

57.-62. (canceled)

63. The method of claim 47, wherein the one or more stimuli comprises contacting the one or more neuronal cells and/or the one or plurality of tissue explants with at least one pharmacologically active compound, electrical stimulus, or chemical stimulus.

64. A method of evaluating the toxicity of an agent comprising:
(a) culturing one or more neuronal cells and/or one or more tissue explants in the composition of claim 27;
(b) exposing at least one agent to the one or more neuronal cells and/or one or more tissue explants;
(c) measuring and/or observing one or more morphometric changes of the one or more neuronal cells and/or one or more tissue explants; and
(d) correlating one or more morphometric changes of the one or more neuronal and/or one or more tissue explants cells with the toxicity of the agent, such that, if the morphometric changes are indicative of decreased cell viability, the agent is characterized as toxic and, if the morphometric changes are indicative of unchanged or increased cell viability, the agent is characterized as non-toxic.

65.-72. (canceled)

73. A method of measuring myelination or demyelination of one or more axons of one or a plurality of neuronal cells and/or one or a plurality of tissue explants, said method comprising:
(a) culturing one or more neuronal cells and/or one or a plurality of tissue explants in the composition of claim 27 for a time and under conditions sufficient to grow at least one axon; and
(b) detecting the amount of myelination on one or a plurality of axons of the one or more neuronal cells and/or one or more tissue explants.

74. (canceled)

75. The method of claim 47 further comprising: (i) exposing one or a plurality of neuronal cells and/or one or a plurality of tissue explants to at least one agent after steps (a) and (b); (ii) measuring and/or observing one or more electrophysiological metrics, measuring and/or observing one or more morphometric changes and/or detecting the quantitative amount of myelin from the one or a plurality of neuronal cells and/or one or a plurality of tissue explants; (iii) calculating a change of measurements, observations and/or quantitative amount of myelin from the one or a plurality of neuronal cells and/or the one or a plurality of tissue explants in the presence and absence of the agent; and (iv) correlating the change of measurements, observations and/or quantitative amount of myelin from the one or a plurality of neuronal cells and/or the one or a plurality of tissue explants to the presence or absence of the agent.

76.-79. (canceled)

80. A method of measuring myelination or demyelination of one or more axons of one or a plurality of neuronal cells and/or one or a plurality of tissue explants, said method comprising:
(a) culturing one or more neuronal cells and/or one or a plurality of tissue explants in the composition of claim 27 for a time and under conditions sufficient to grow at least one axon; and
(b) inducing a compound action potential in such one or more neuronal cells and/or one or more tissue explants;
(c) measuring the compound action potential; and
(d) quantifying the levels of myelination of such one or more neuronal cells based on the compound action potential.

81.-91. (canceled)

92. A method of detecting and/or quantifying neuronal cell growth or degeneration comprising:
(a) quantifying one or a plurality of neuronal cells;
(b) culturing the one or more neuronal cells in the composition of claim 27; and
(c) calculating the number of neuronal cells in the composition after a culturing for a time period sufficient to allow growth or degeneration of the one or plurality of cells; or
(c) quantifying the number and/or the density of the axons grown from neuronal cells after contacting the one or plurality of cells to one or a plurality of agents; and calculating a difference in the number or density of axons in culture in the presence or absence of the agent.

93.-95. (canceled)

96. A method of detecting or quantifying of axon degeneration of one or a plurality of neuronal cells comprising:
(a) seeding one or a plurality of neuronal cells in a composition of claim 27;
(b) culturing the one or plurality of neuronal cells for a time period and under conditions sufficient to grow at least one or a plurality of axons from the one or plurality of neuronal cells,
(c) quantifying the number or density of axons grown from the neuronal cells;
(d) contacting the one or plurality of neuronal cells to one or a plurality of agents;
(e) quantifying the number and/or the density of the axons grown from neuronal cells after contacting the one or plurality of cells to one or a plurality of agents; and
(f) calculating a difference in the number or density of axons in culture in the presence or absence of the agent.

97.-106. (canceled)

107. A method of measuring or quantifying any neuromodulatory effect of an agent comprising:
(a) culturing one or a plurality of neuronal cells or tissue explants in a composition of claim 27 in the presence and absence of the agent;
(b) applying a voltage potential across the one or a plurality of neuronal cells or tissue explants in the presence and absence of the agent;
(c) measuring one or a plurality of electrophysiological metrics from the one or plurality of neuronal cells or tissue explants in the presence and absence of the agent; and
(d) correlating the difference in one or a plurality of electrophysiological metrics through the one or plurality of neuronal cells or tissue explants to the neuromodulatory effect of the agent, such that a change in electrophysiological metrics in the presence of the agent as compared to the electrophysiological metrics measured in the absence of the agent is indicative of a neuromodulatory effect, and no change of electrophysiological metrics in the presence of the agent as compared to the electrophysiological metrics measured in the absence of the agent is indicative of the agent not conferring a neuromodulatory effect.

108.-113. (canceled)
